(19) United States
(12) Patent Application Publication
(10) Pub. No.: US 2012/0156723 A1
(43) Pub. Date: Jun. 21, 2012

(54) PROTEIN GLYCOSYLATION

(76) Inventors: Brendan Wren, St. Albans (GB); Rebecca Langdon, Brighouse (GB)

(21) Appl. No.: 13/394,073

(22) PCT Filed: Sep. 3, 2010
(86) PCT No.: PCT/GB2010/001665

§ 371 Date: Mar. 2, 2012

(30) Foreign Application Priority Data
Sep. 4, 2009 (GB) 0915403.0

(51) Int. Cl. C12P 21/00; C12N 1/21

(52) U.S. Cl. 435/69.3; 435/252.3; 435/252.33

(57) ABSTRACT

The disclosure relates to an oligosaccharyltransferase polypeptide and the production of glycosylated recombinant protein in a microbial host cell; and including vaccines comprising glycosylated recombinant antigens.

The invention relates to an oligosaccharyltransferase polypeptide and its use in the production of glycosylated recombinant protein by a microbial host cell and including vaccines comprising glycosylated recombinant antigens.

### INTRODUCTION

The large scale production of recombinant proteins, for example enzymes, polypeptide hormones, recombinant monoclonal antibodies and recombinant antigens, requires a high standard of quality control since many of these proteins are administered to humans. Moreover, the development of vaccines, particularly subunit vaccines, requires the production of large amounts of pure protein free from contaminating antigens which may provoke anaphylaxis. The production of recombinant protein in cell expression systems is based either on prokaryotic cell or eukaryotic cell expression. The latter is preferred when post-translation modifications, for example glycosylation, to the protein are required.

### BACKGROUND

Glycosylation is the addition of a sugar pendent group to a protein, polypeptide or peptide which alters the activity and/or bioavailability of the protein, polypeptide or peptide. The process is either co-translational or post-translational and is enzyme mediated. Two types of glycosylation exist; N-linked glycosylation to an asparagine side chain and O-linked glycosylation to a serine or threonine amino acid side chain. N-linked glycosylation is the most common post-translational modification and is carried out in the endoplasmic reticulum of eukaryotic cells. N-linked glycosylation can be of two main types; high mannose oligosaccharides which are two N-acetylglucosamines and complex oligosaccharides which include other types of sugar groups. A peptide motif contained in glycosylated polypeptides is Asn-X-Ser or Asn-X-Thr where X is any amino acid except proline. This is catalyzed by the enzyme oligosaccharyl transferase [OT]; see Yan & Lennarz J. Biol. Chem., Vol. 280 (5), 3121-3124 (2005) OT catalyzes the transfer of an oligosaccharyl moiety (Glc3Man9GlcNAc2) from the dolichol-linked pyrophosphate donor to the side chain of an Asn. A pentasaccharide core is common to all N-linked oligosaccharides and serves as the foundation for a wide variety of N-linked oligosaccharides. O-linked glycosylation is less common. Serine or threonine residues are linked via their side chain oxygen to sugars by a glycosidic bond. Usually N-acetyl glucosamine is attached in this way to intracelluar proteins.

Only recently has it been recognized that prokaryotic cells have the capability to glycosylate protein. In particular, it is recognized that N-linked glycosylation among some ε proteobacteria is present. For example, the Campylobacter jejuni [C. jejuni] genome encodes genes involved in the synthesis of lipo-oligosaccharides and N linked glycoproteins. The protein glycosylation locus [pgl locus] is involved in the glycosylation of over 30 glycoproteins. It has also been demonstrated that the pgl genes can function in Escherichia coli [E. coli] to modify co-expressed C. jejuni proteins which suggests that E. coli may be engineered to produce heterologous recombinant glycoproteins. The C. jejuni locus-encoded PglB can transfer alternative glycans, including bacterial O-antigens, to mature N-linked heptasaccharide GalNac5GlcBac implying relaxed specificity. However there are problems associated with this enzyme; the enzyme is unable to transfer all glycans. This may result from a requirement of an acetamido group at the C2 position in the sugar at the reducing end of the glycan. There is therefore a desire to identify alternate oligosaccharyltransferases that are not so encumbered.

One of the most important discoveries in the history of medicine is the development of vaccination strategies which are used to protect humans and animals against a wide variety of infectious and non-infectious diseases. Many vaccines are produced by inactivated or attenuated pathogens which are injected into an individual. These can sometimes cause adverse side effects. Many modern vaccines are made from protective antigens of the pathogen, separated by purification or molecular cloning from the materials that give rise to side-effects. These latter vaccines are known as ‘subunit vaccines’. Bacterial infections caused by encapsulated bacteria are a major world health problem. The species Streptoccocus pneumoniae, Haemophilus influenzae and Neisseria meningitidis are difficult to vaccinate against due to the T cell independent nature of the major surface antigens, the capsular polysaccharides. T-cell independent antigens, for example capsular polysaccharides, present particular problems regarding the development of effective vaccines. Antibody production is low and is not normally boosted by re-immunisation. The antibody isotypes are restricted to the IgM and other isotypes are generally of a low affinity for a specific antigen. A major problem lies in the response of young children to T-cell independent vaccines. These individuals are amongst the most vulnerable to bacterial infections.

T-cell dependent antigens are much more effective at eliciting high titre, high affinity antibody responses and are typically proteins. This is because T-lymphocyte help to B-lymphocytes is elicited during the immune response to these antigens. B-Lymphocytes bind to antigen through their specific antigen receptors which leads to partial activation. If the antigen is a protein the B-lymphocytes take up and process the antigen to peptides which are expressed on the cell surface along with HLA class II molecules. T-cell independent antigens are invariably not protein in composition and cannot therefore be processed and presented by B-lymphocytes via HLA molecules. This failure in antigen presentation results in low T-cell recognition of the antigen thereby resulting in no T-cell help.

Glycoconjugate vaccines for Streptococcus pneumoniae, Neisseria meningitidis and Haemophilus influenzae are currently licensed for human use and are produced by linking the capsule (or other bacterial glycan-based structure such as lipooligosaccharide, LOS) from these bacteria to a protein toxoid. Whilst these vaccines provide a good level of immunity they are expensive and difficult to produce, requiring the purification of the glycan from the pathogenic organisms and chemical linkage to the carrier protein. There is also evidence that disease caused by serotypes not covered by the vaccines is emerging. The use of organic systems represents a more rapid and economical method for the production of glycoconjugates.

This disclosure relates to the identification and characterisation of a oligosaccharyltransferase homologous to C. jejuni PglB and which is able to glycosylate proteins without the requirement for a acetamido group thereby providing protein glycoconjugates useful in vaccines that will benefit from T cell help to provide effective vaccines to, for example, bacterial infections.

### STATEMENTS OF INVENTION

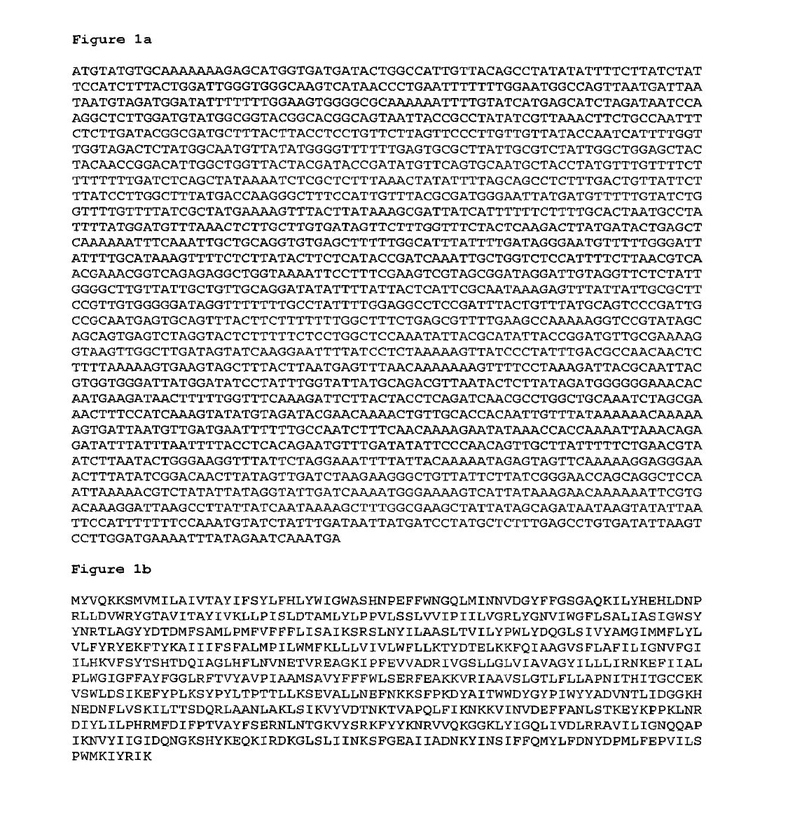

According to an aspect of the invention there is provided a microbial cell transformed with a vector comprising a nucleotide sequence selected from the group consisting of
i) a nucleic acid molecule consisting of a nucleic acid sequence as represented in FIG. 1a;
ii) a nucleic acid molecule consisting of a nucleic acid sequence that hybridises under stringent hybridisation conditions to the nucleic acid molecule in (i) and which encodes an polypeptide; wherein said cell expresses a recombinant polypeptide which is a substrate for said oligosaccharyltransferase polypeptide.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×-3×SSC at RT to 55° C. for 20-30 minutes each.

In a preferred embodiment of the invention said microbial cell is transformed with a nucleic acid molecule comprising a nucleotide sequence that encodes an oligosaccharyltransferase polypeptide as represented by the amino acid sequence in FIG. 1b, or a variant polypeptide and comprises the amino acid sequence represented in FIG. 1b which sequence has been modified by deletion, addition or substitution of at least one amino acid residue and which retains or has enhanced oligosaccharyltransferase activity.

A variant polypeptide may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies.

In addition, the invention features polypeptide sequences having at least 40-75% identity with the polypeptide sequence as herein disclosed, or fragments and functionally equivalent polypeptides thereof; preferably at least 43% identity over the entire amino acid sequence. In one embodiment, the polypeptides have at least 85% identity, more preferably at least 90% identity, even more preferably at least 95% identity, still more preferably at least 97% identity, and most preferably at least 99% identity with the amino acid sequence over the entire amino acid sequence illustrated herein with reference to FIG. 1b.

In a preferred embodiment of the invention said vector is an expression vector adapted for expression of a nucleic acid molecule encoding the oligosaccharyltransferase polypeptide.

There is a significant amount of published literature with respect to expression vector construction and recombinant DNA techniques in general. Please see, Sambrook et at (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

In a preferred embodiment of the invention said recombinant polypeptide includes at least one peptide motif consisting of the amino acid sequence:

Asp/Glu-Xaa₁-Asn-Xaa₂-Ser/Thr

wherein Xaa₁ and Xaa₂ is any amino acid except proline.

In a preferred embodiment of the invention said recombinant polypeptide is an antigen isolated from an infectious agent.

In a preferred embodiment of the invention said infectious agent is a bacterial pathogen.

Preferably, the polysaccharides will be transferred to proteins from the following: Streptococcus pneumoniae, Streptococcus suis, Streptococcus inae, Streptococcus agalactiae, Streptococcus dysgalactiae, Streptococcus equi, Streptococcus uberist, Streptococcus milleri group (SMG), Streptococcus sanguis, Streptococcus bovis, Streptococcus group A, Klebsiella pneumoniae, Klebsiella oxytoca, Klebsiella planticola, Pseudomonas aerunginosa, Acinetobcater baumanii, Acinetobacter calcoaceticus, Salmonella enterica serovar Typhi, Campylobacter jejuni, Campylobacter coli, Campylobacter lari, Haemophilus influenzae, Helicobacter pylori, Neisseria meningitidis, Neisseria gonorrhoea, Neisseria lactamica, Shigella spp., Staphylococcus spp, Clostridium difficile, Clostridium botulinum, Bacillus anthracis, Burkholderia pseudomallei, Burkholderia mallei Lactobacillus spp., Clostridium tetani, Corynebacterium diptheriae, Vibrio cholerae, Escherichia coli, Mycobacterium tuberculosis, Bordetella pertussis, Actinobacillus pleuropneumoniae, virally derived antigens, for example Human papillomavirus (HPV), Hepatitis A, Hepatitis B, Rotavirus and Influenza virus, antigen derived from parasites, for example, Plasmodium faciparum.

In a preferred embodiment of the invention said bacterial pathogen is selected from the group consisting of: Staphylococcus epidermidis, S. aureus, S. hominis, S. haemolyticus, S. wameri, S. capitis, S. saccharolyticus, S. auricularis, S. simulans, S. saprophyticus, S. cohnii, S. xylosus, S. cohnii, S. wameri, S. hyicus, S. caprae, S. gallinarum, S. intermedius, S. hominis. The oligosaccharyltransferase polypeptide according to the invention will be used to conjugate polysaccharides from the following:
A) Capsular (K) antigens from Streptococcus pneumoniae, Streptococcus suis, Streptococcus inae, Streptococcus agalactiae, Streptococcus milleri group (SMG), Streptococcus bovis, Klebsiella pneumoniae, Klebsiella oxytoca, Klebsiella planticola, Pseudomonas aerunginosa, Acinetobcater baumannii, Acinetobacter calcoaceticus, Salmonella enterica serovar Typhi, Burkholderia pseudomallei, Burkholderia mallei, Cryptococcus neoformans, Campylobacter jejuni, Actinobacillus pleuropneumoniae, Mycoplasma mycoides subsp. mycoidesSC, Lactococcus garvieae
B) O-antigens from Klebsiella pneumoniae, Klebsiella oxytoca, Klebsiella planticola, Pseudomonas aerunginosa, Acinitobcater baumanii, Salmonella enterica serovar Typhimurium, Salmonella enterica serovar Typhi, Yersinia pseudotuberculosis, Brucella spp., Vibrio cholerae, Actinobacillus pleuropneumoniae, Serratia marcescens
C) Exopolysaccharide/surface polysaccharides from Psuedomonas aeruginosa, Mycobacterium tuberculosis

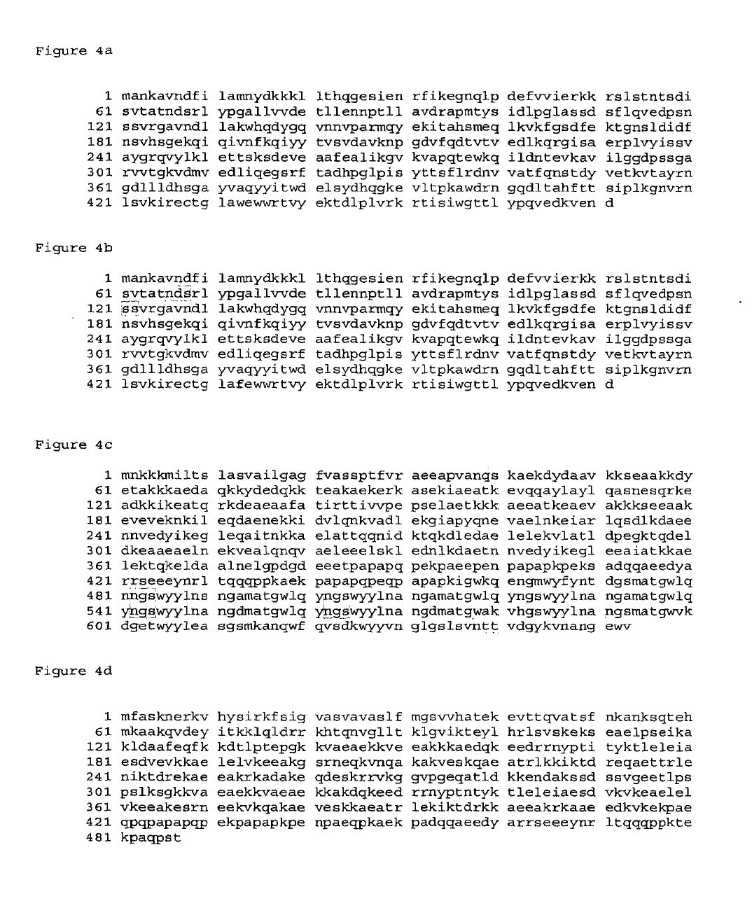

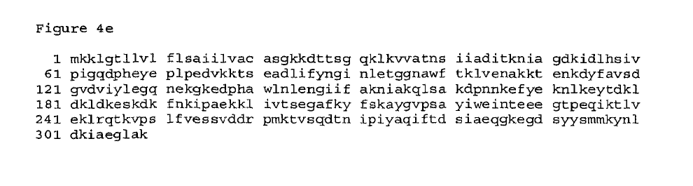

In a preferred embodiment of the invention said recombinant antigenic polypeptide comprises or consists of an amino acid sequence, or antigenic part thereof, selected from the group consisting of the sequences represented in FIGS. 4a-4e, or a variant amino acid sequence wherein said variant is the deletion, substitution or addition of at least one amino acid residue represented in FIGS. 4a-4e.

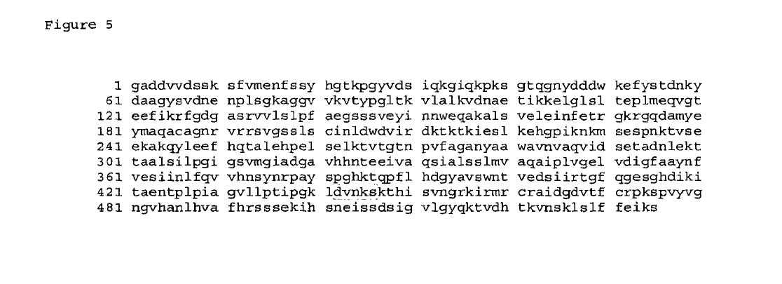

In a preferred embodiment of the invention said recombinant antigenic polypeptide comprises or consists of an amino acid sequence, or antigenic part thereof, selected from the group consisting of the sequences represented in FIG. 5a or a variant amino acid sequence wherein said variant is the deletion, substitution or addition of at least one amino acid residue represented in FIG. 5.

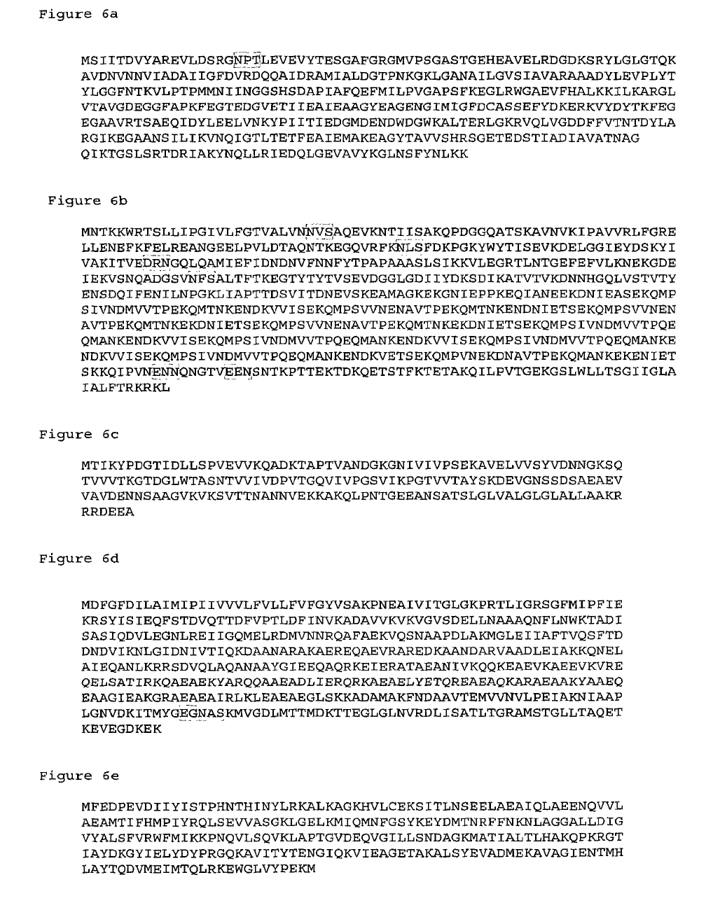

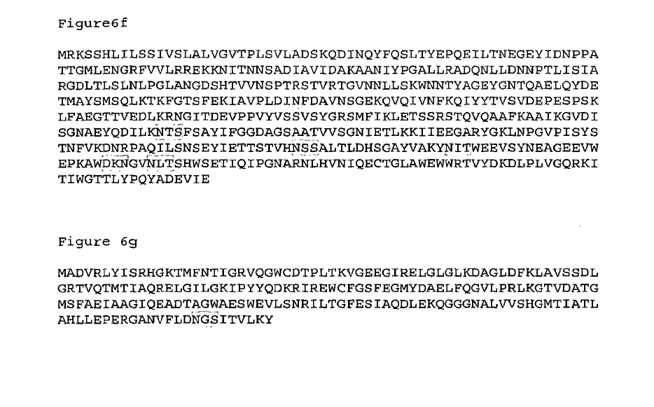

In a preferred embodiment of the invention said recombinant antigenic polypeptide comprises or consists of an amino acid sequence, or antigenic part thereof, selected from the group consisting of the sequences represented in FIGS. 6a-6g, or a variant amino acid sequence wherein said variant is the deletion, substitution or addition of at least one amino acid residue represented in FIGS. 6a-6g.

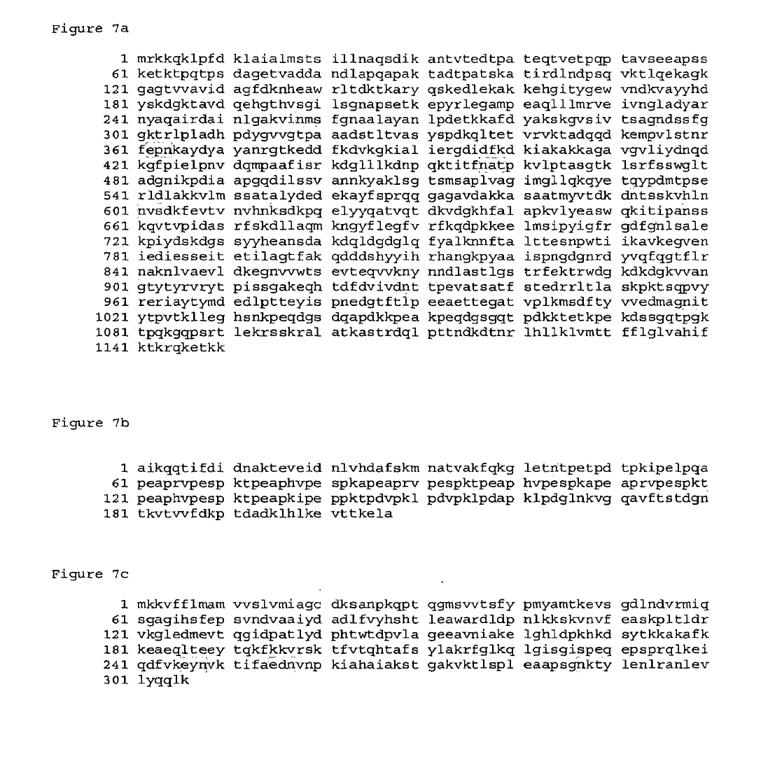

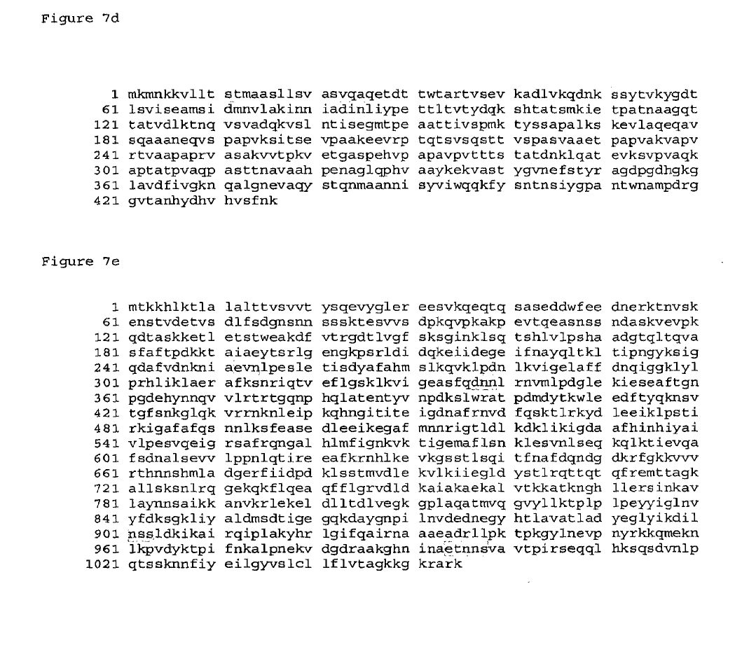

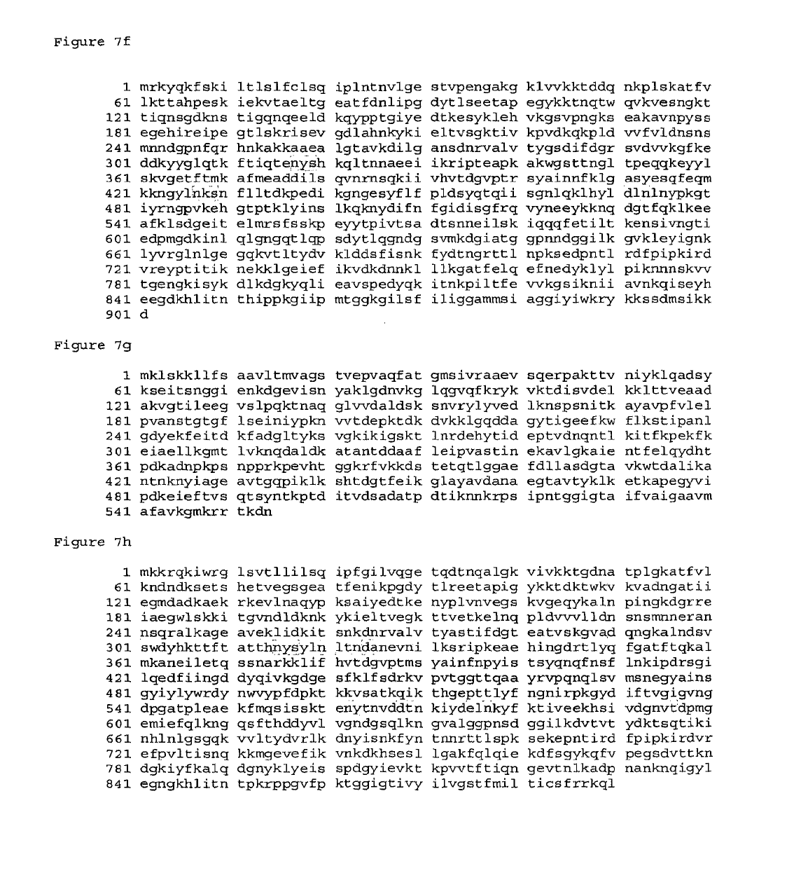

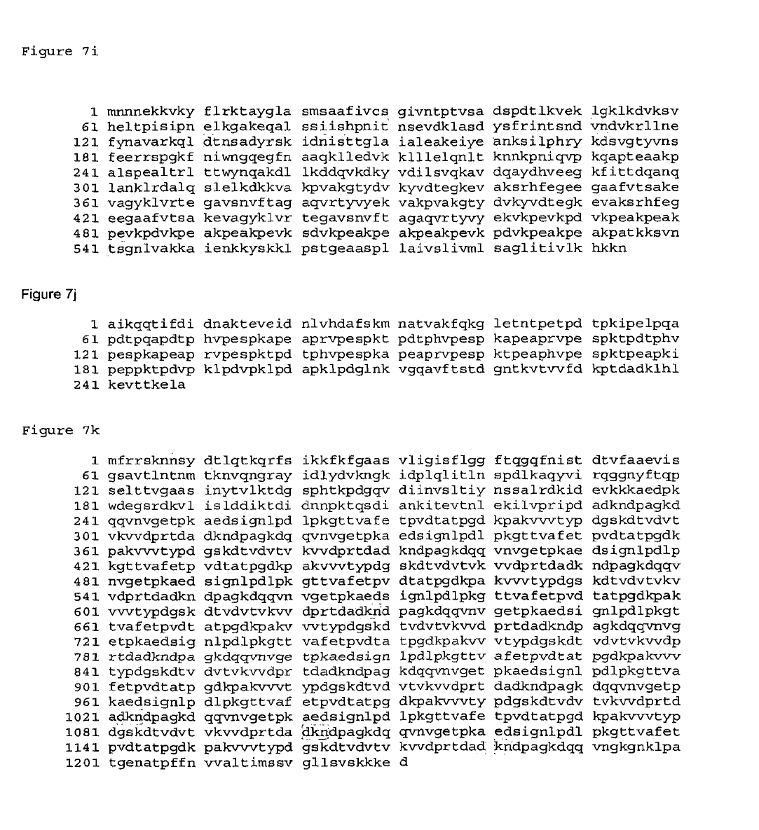

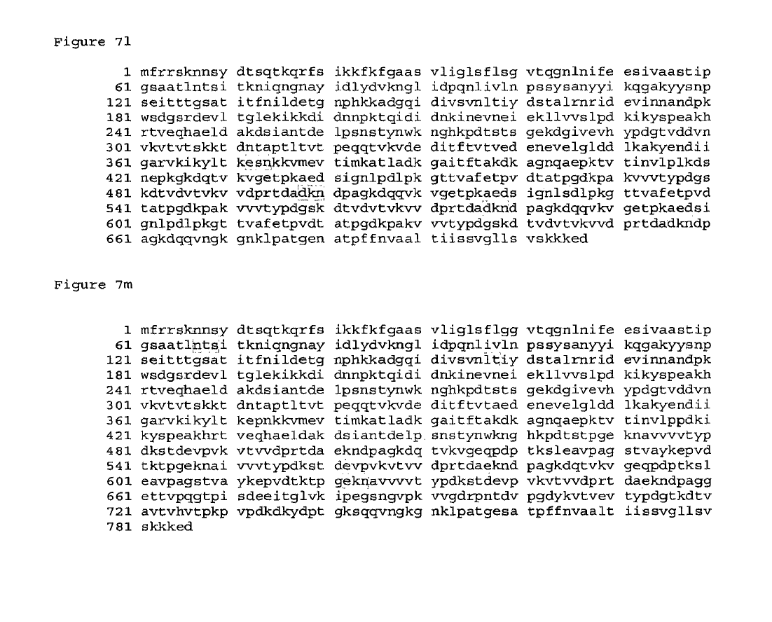

In a preferred embodiment of the invention said recombinant antigenic polypeptide comprises or consists of an amino acid sequence, or antigenic part thereof selected from the group consisting of the sequences represented in FIGS. 7a-7m, or a variant amino acid sequence wherein said variant is the deletion, substitution or addition of at least one amino acid residue represented in FIGS. 7a-7m.

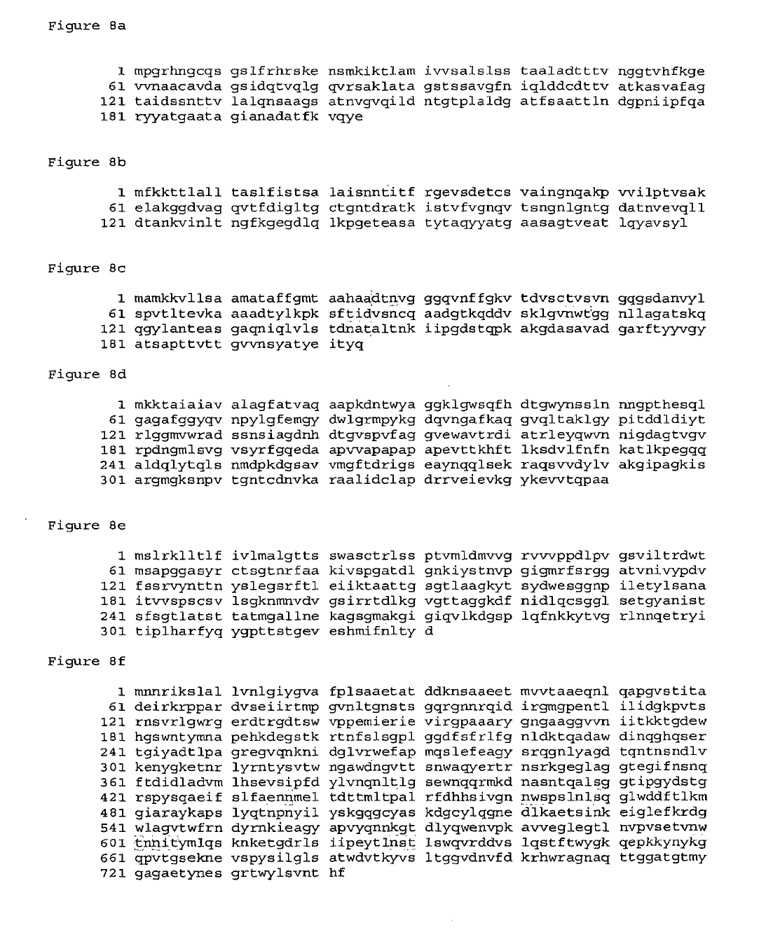

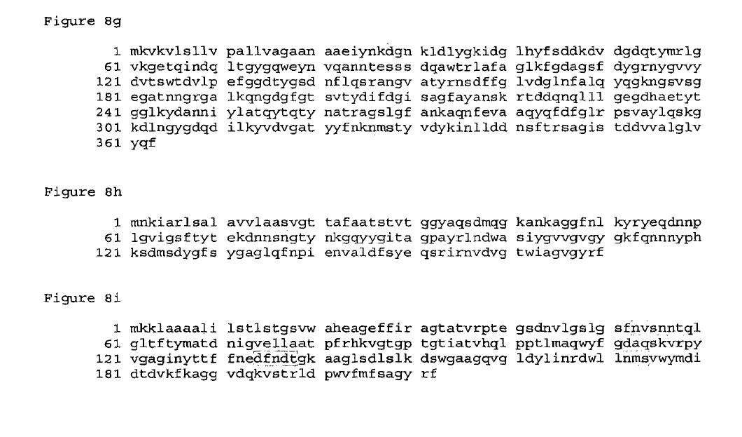

In a preferred embodiment of the invention said recombinant antigenic polypeptide comprises or consists of an amino acid sequence, or antigenic part thereof, selected from the group consisting of the sequences represented in FIGS. 8a-8i, or a variant amino acid sequence wherein said variant is the deletion, substitution or addition of at least one amino acid residue represented in FIGS. 8a-8i.

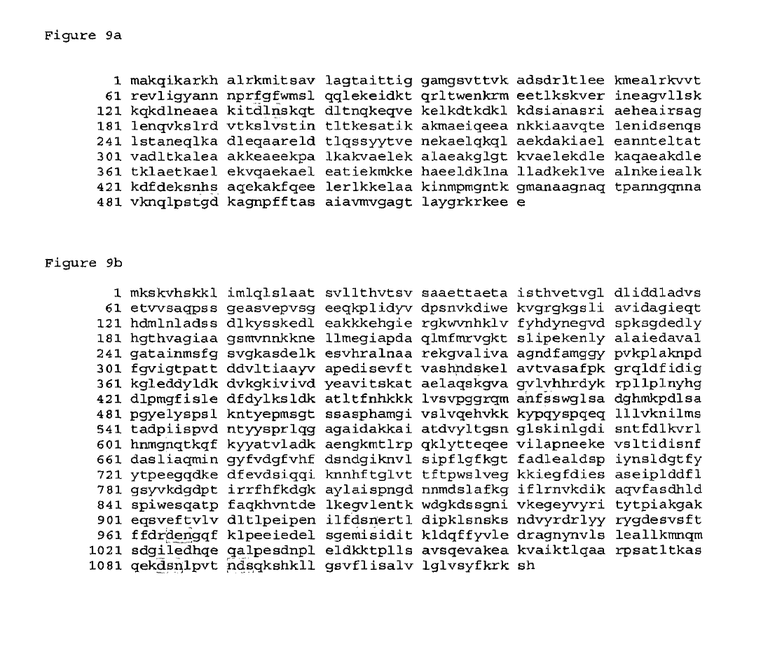

In a preferred embodiment of the invention said recombinant antigenic polypeptide comprises or consists of an amino acid sequence, or antigenic part thereof, selected from the group consisting of the sequences represented in FIG. 9a or 9b, or a variant amino acid sequence wherein said variant is the deletion, substitution or addition of at least one amino acid residue represented in FIG. 9a or 9b.

In a preferred embodiment of the invention said recombinant antigenic polypeptide comprises or consists of an amino acid sequence, or antigenic part thereof, selected from the group consisting of the sequences represented in FIG. 10a or 10b, or a variant amino acid sequence wherein said variant is the deletion, substitution or addition of at least one amino acid residue represented in FIG. 10a or 10b.

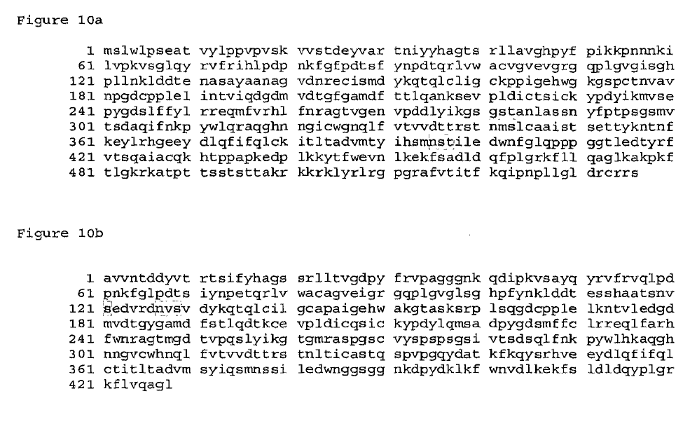

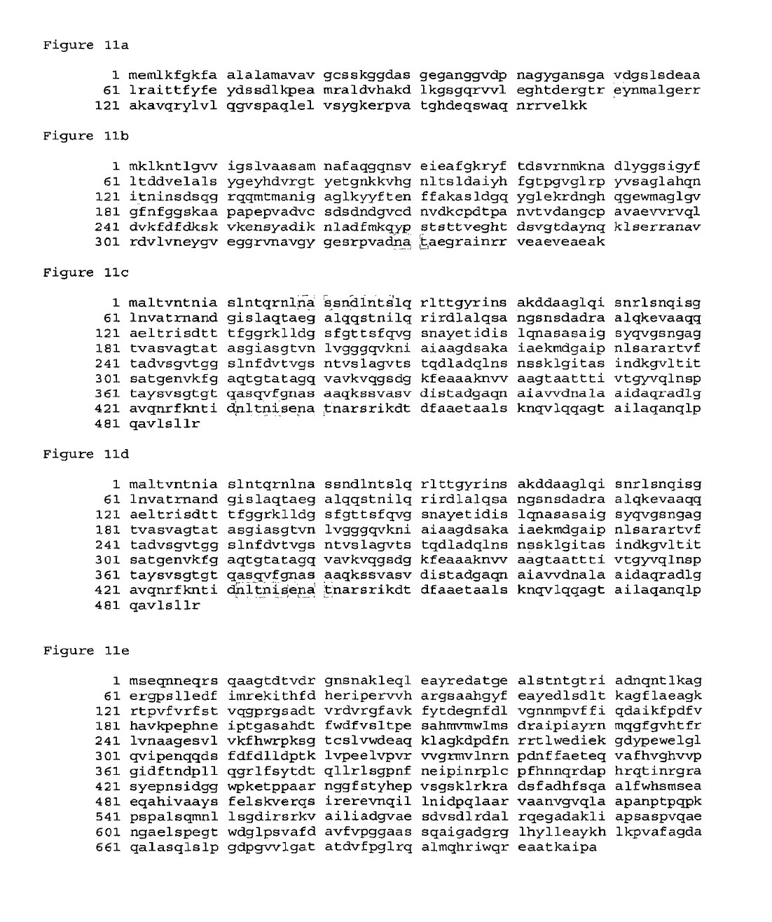

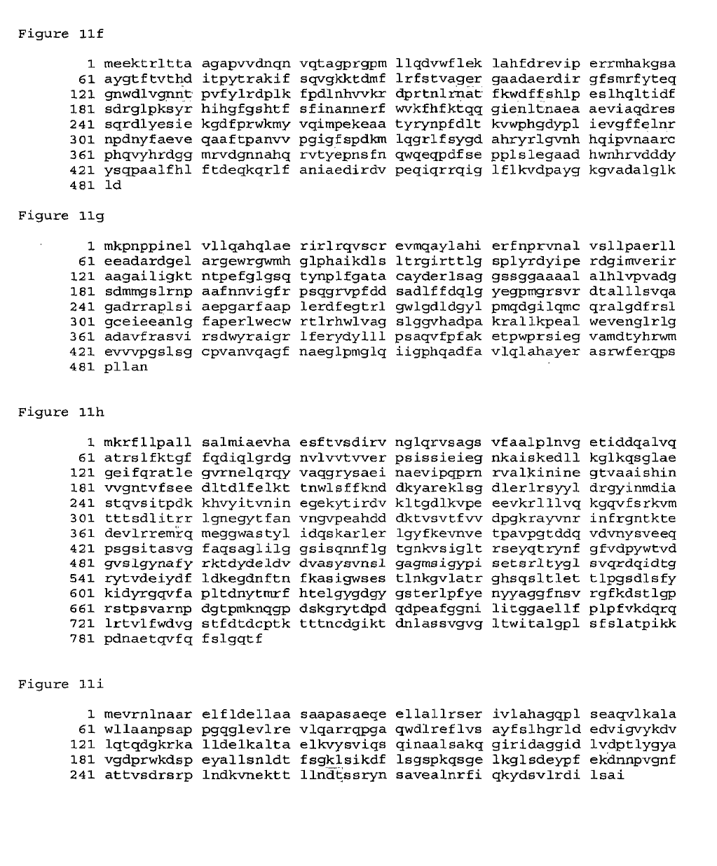

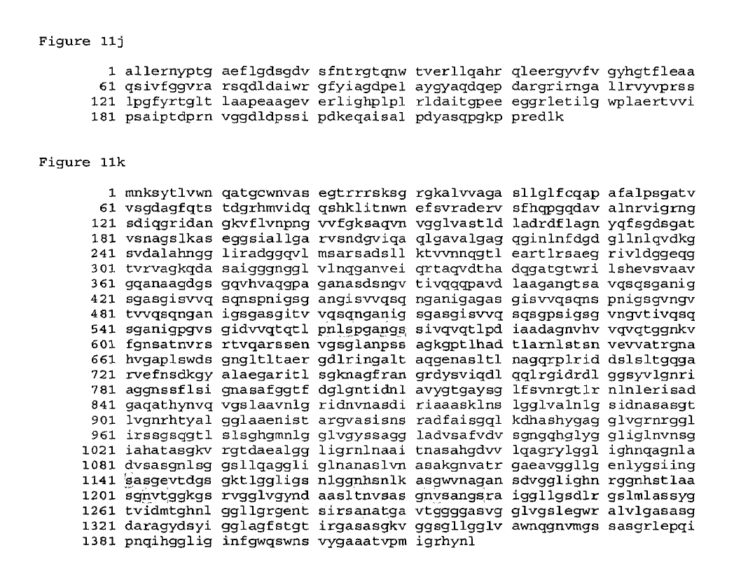

In a preferred embodiment of the invention said recombinant antigenic polypeptide comprises or consists of an amino acid sequence, or antigenic part thereof, selected from the group consisting of the sequences represented in FIGS. 11a-11k, or a variant amino acid sequence wherein said variant is the deletion, substitution or addition of at least one amino acid residue represented in FIGS. 11a-11k.

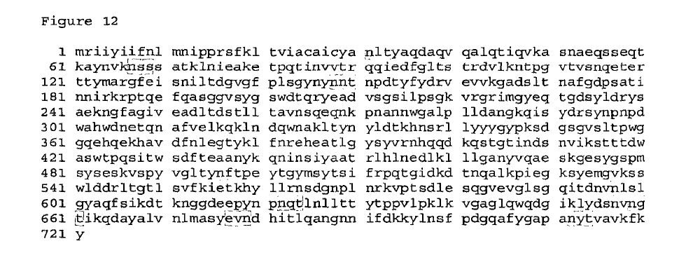

In a preferred embodiment of the invention said recombinant antigenic polypeptide comprises or consists of an amino acid sequence, or antigenic part thereof, represented in FIG. 12, or a variant amino acid sequence wherein said variant is the deletion, substitution or addition of at least one amino acid residue represented in FIG. 12.

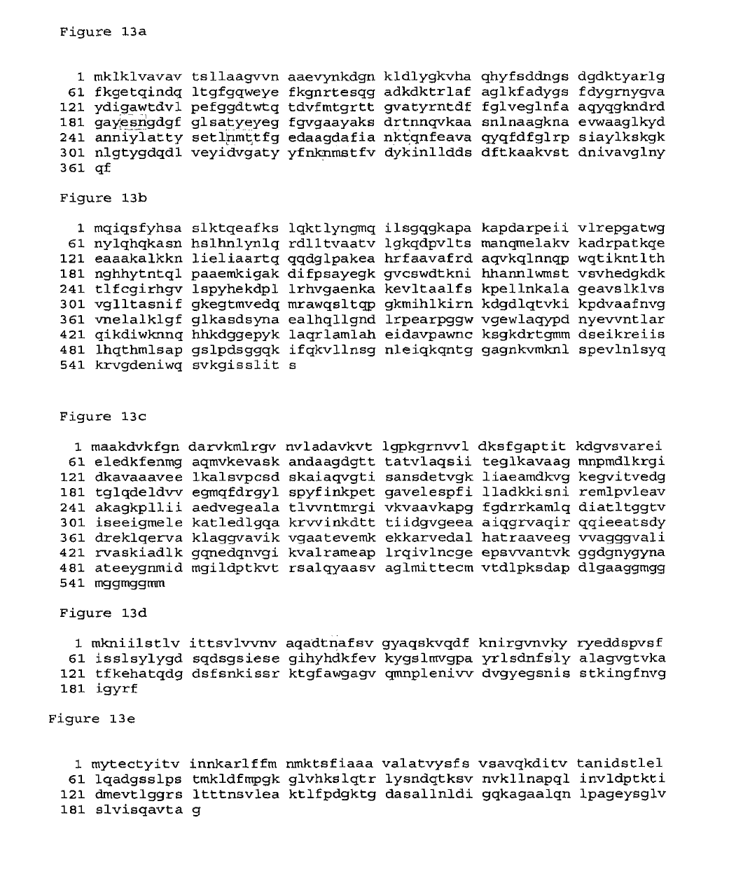

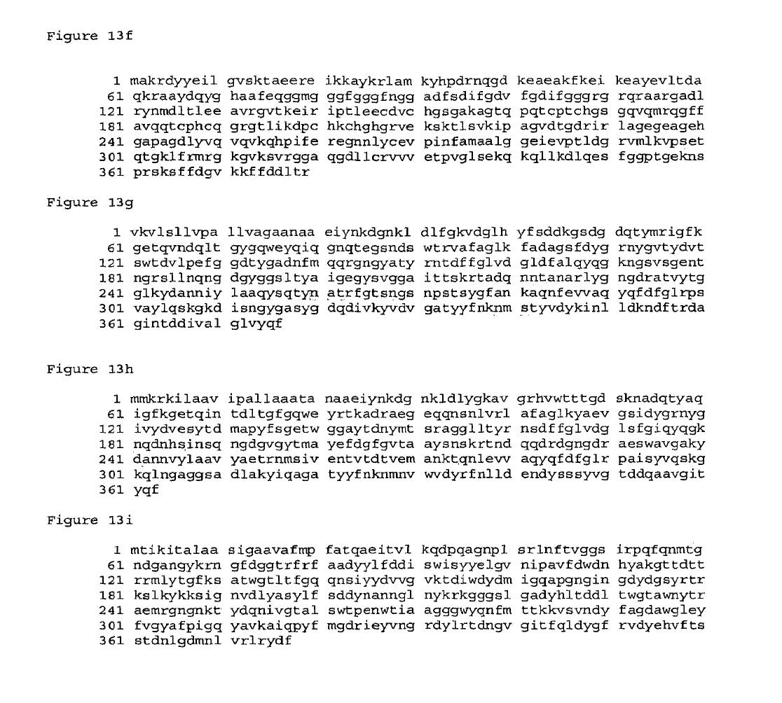

In a preferred embodiment of the invention said recombinant antigenic polypeptide comprises or consists of an amino acid sequence, or antigenic part thereof, selected from the group consisting of the sequences represented in FIGS. 13a-13l, or a variant amino acid sequence wherein said variant is the deletion, substitution or addition of at least one amino acid residue represented in FIGS. 13a-13l.

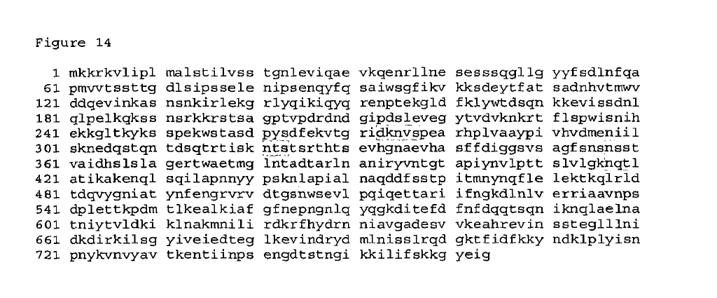

In a preferred embodiment of the invention said recombinant antigenic polypeptide comprises or consists of an amino acid sequence, or antigenic part thereof, represented in FIG. 14, or a variant amino acid sequence wherein said variant is the deletion, substitution or addition of at least one amino acid residue represented in FIG. 14.

In a preferred embodiment of the invention said recombinant antigenic polypeptide comprises or consists of an amino acid sequence, or antigenic part thereof, selected from the group consisting of the sequences represented in FIGS. 15a-15f, or a variant amino acid sequence wherein said variant is the deletion, substitution or addition of at least one amino acid residue represented in FIGS. 15a-15f.

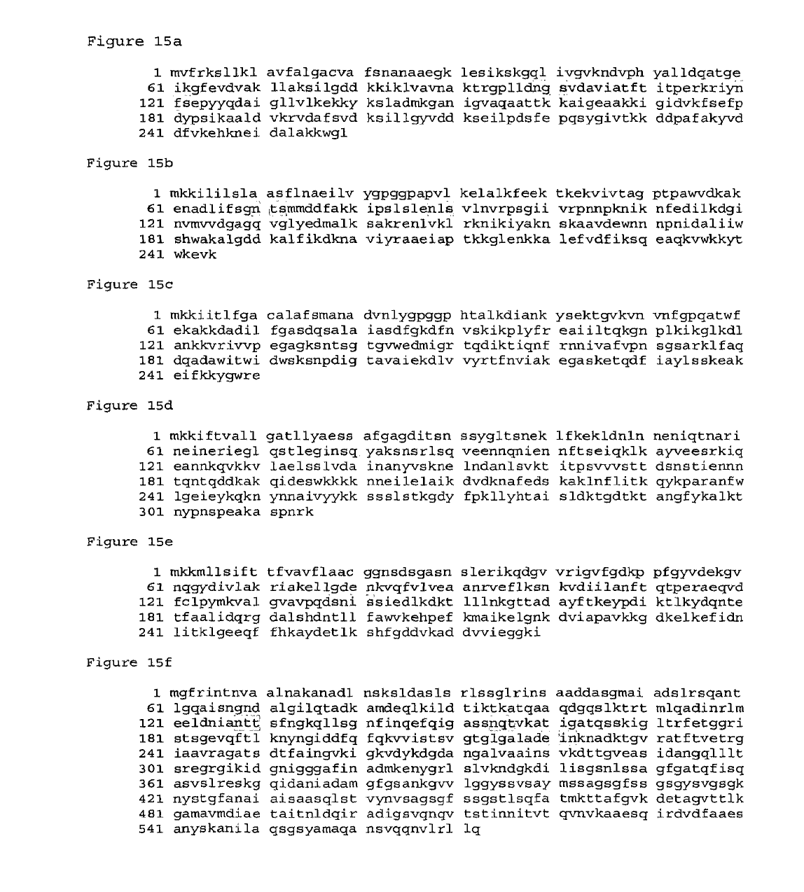

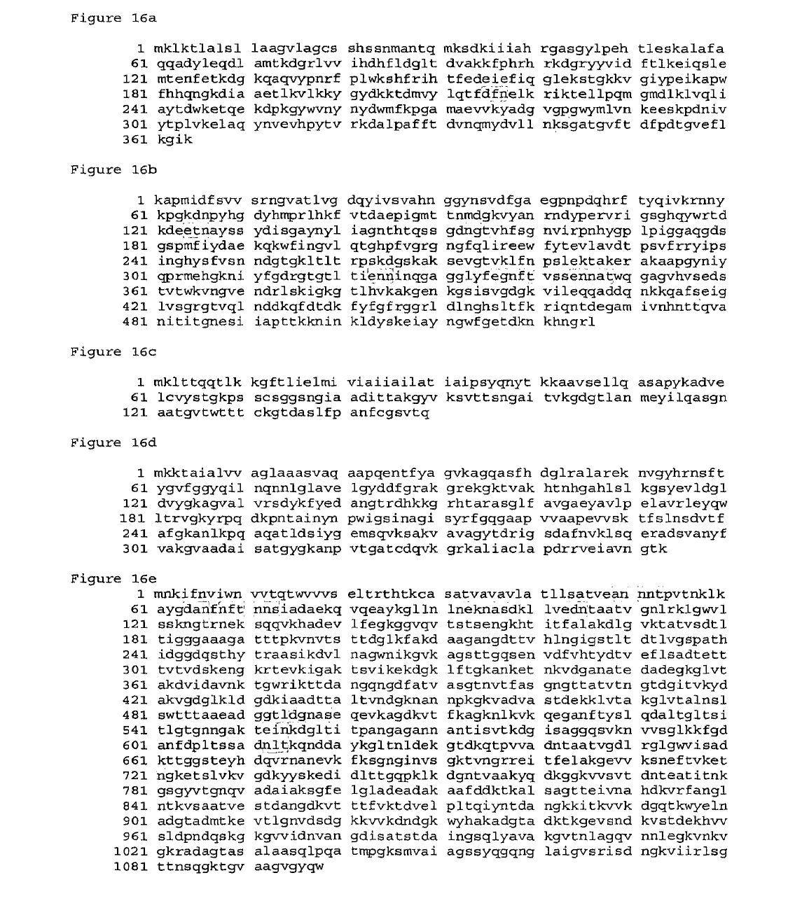

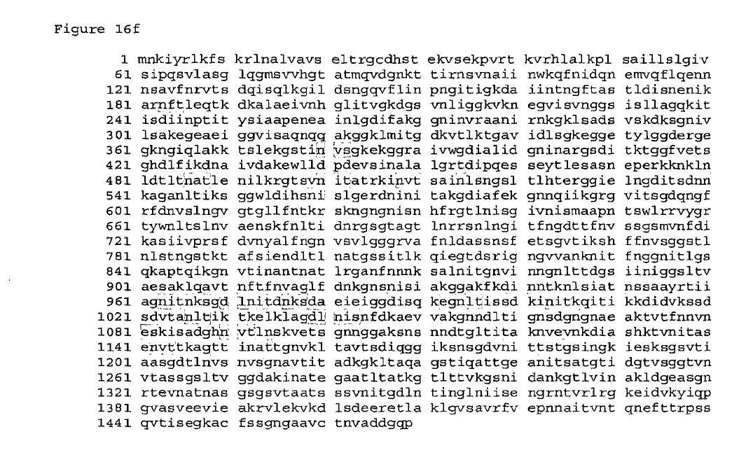

In a preferred embodiment of the invention said recombinant antigenic polypeptide comprises or consists of an amino acid sequence, or antigenic part thereof, selected from the group consisting of the sequences represented in FIGS. 16a-16f, or a variant amino acid sequence wherein said variant is the deletion, substitution or addition of at least one amino acid residue represented in FIGS. 16a-16f.

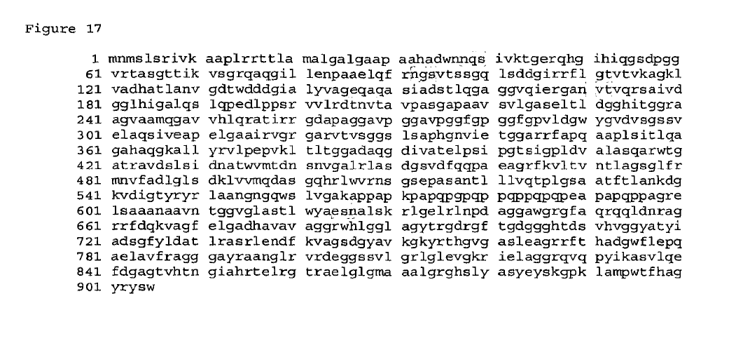

In a preferred embodiment of the invention said recombinant antigenic polypeptide comprises or consists of an amino acid sequence, or antigenic part thereof, represented in FIG. 17, or a variant amino acid sequence wherein said variant is the deletion, substitution or addition of at least one amino acid residue represented in FIG. 17.

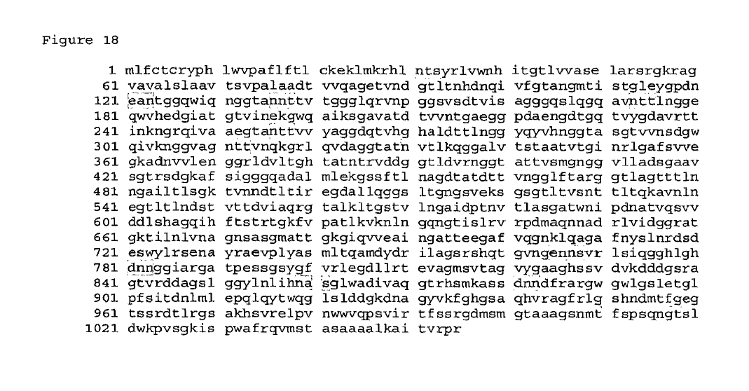

In a preferred embodiment of the invention said recombinant antigenic polypeptide comprises or consists of an amino acid sequence, or antigenic part thereof, represented in FIG. 18, or a variant amino acid sequence wherein said variant is the deletion, substitution or addition of at least one amino acid residue represented in FIG. 18.

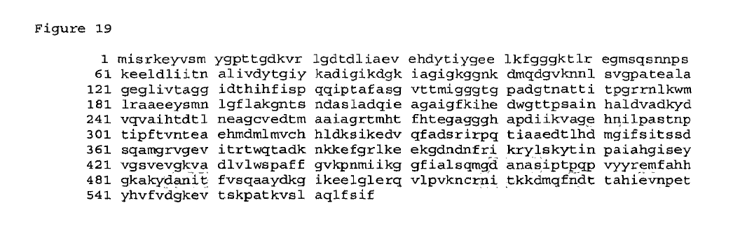

In a preferred embodiment of the invention said recombinant antigenic polypeptide comprises or consists of an amino acid sequence, or antigenic part thereof, represented in FIG. 19, or a variant amino acid sequence wherein said variant is the deletion, substitution or addition of at least one amino acid residue represented in FIG. 19.

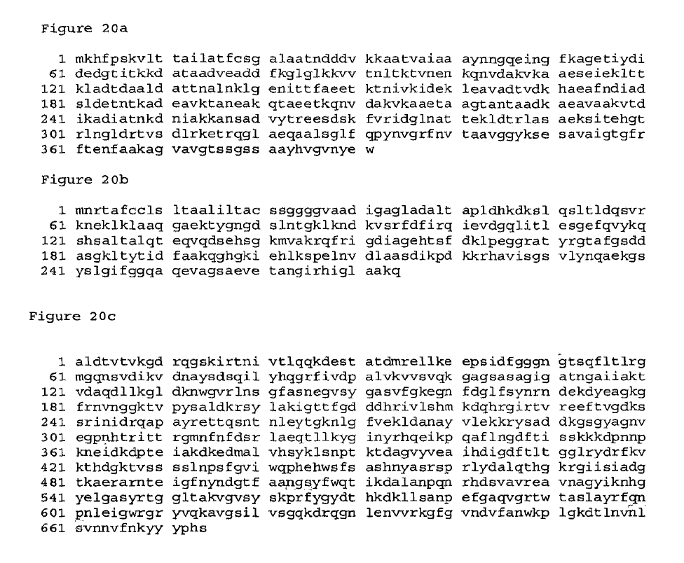

In a preferred embodiment of the invention said recombinant antigenic polypeptide comprises or consists of an amino acid sequence, or antigenic part thereof, selected from the group consisting of the sequences represented in FIGS. 20a-20c, or a variant amino acid sequence wherein said variant is the deletion, substitution or addition of at least one amino acid residue represented in FIGS. 20a-20c.

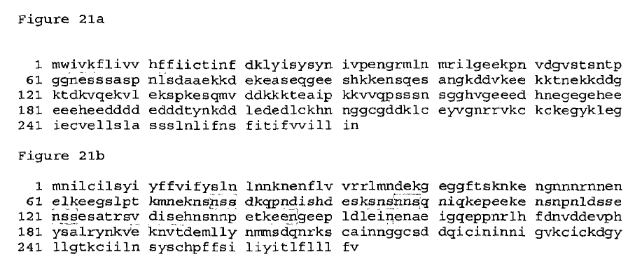

In a preferred embodiment of the invention said recombinant antigenic polypeptide comprises or consists of an amino acid sequence, or antigenic part thereof, selected from the group consisting of the sequences represented in FIG. 21a or 21b, or a variant amino acid sequence wherein said variant is the deletion, substitution or addition of at least one amino acid residue represented in FIG. 21a or 21b.

In a preferred embodiment of the invention said recombinant antigenic polypeptide comprises or consists of an amino acid sequence, or antigenic part thereof, selected from the group consisting of the sequences represented in FIGS. 22a-22f, or a variant amino acid sequence wherein said variant is the deletion, substitution or addition of at least one amino acid residue represented in FIGS. 22a-22f.

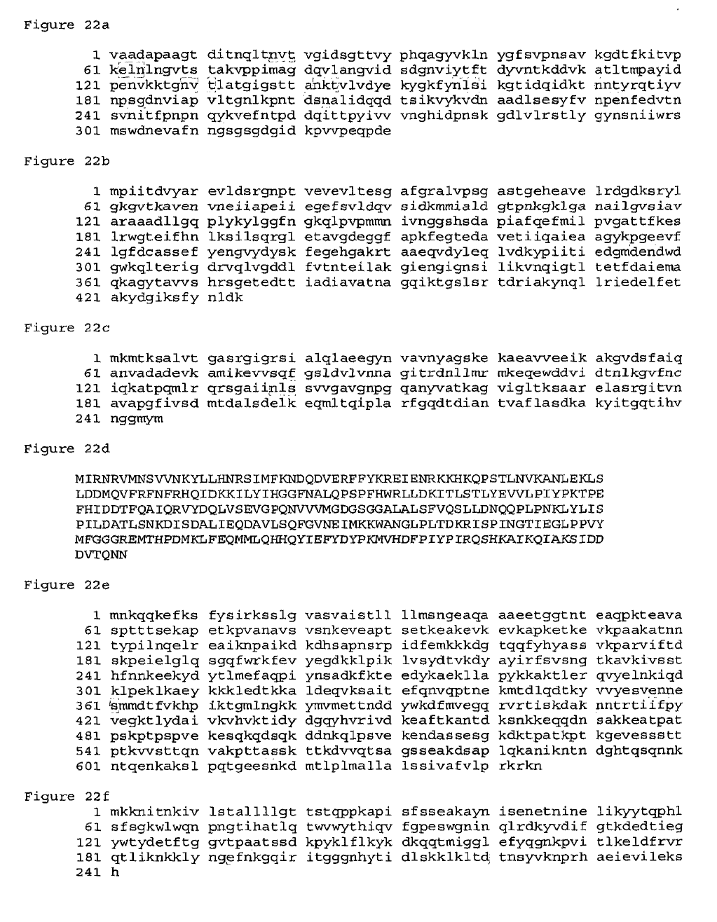

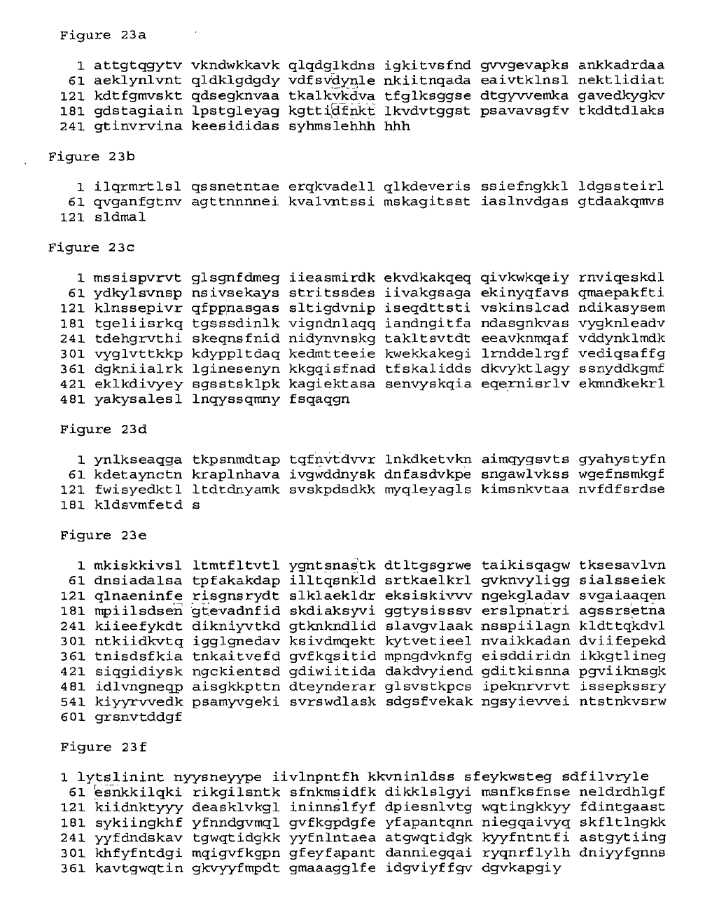

In a preferred embodiment of the invention said recombinant antigenic polypeptide comprises or consists of an amino acid sequence, or antigenic part thereof, selected from the group consisting of the sequences represented in FIGS. 23a-23f, or a variant amino acid sequence wherein said variant is the deletion, substitution or addition of at least one amino acid residue represented in FIGS. 23a-23f.

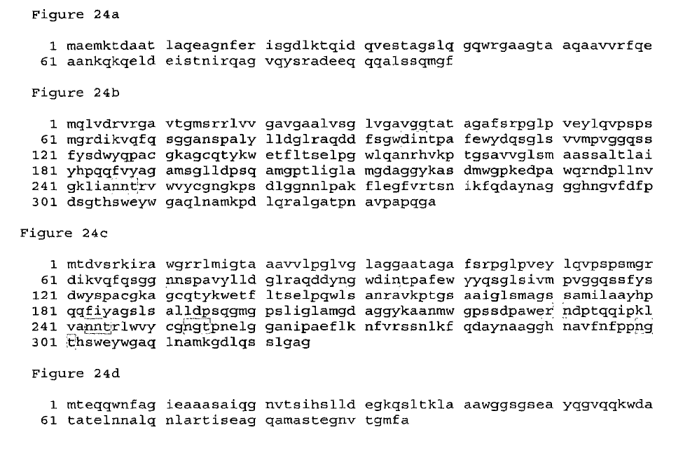

In a preferred embodiment of the invention said recombinant antigenic polypeptide comprises or consists of an amino acid sequence, or antigenic part thereof, selected from the group consisting of the sequences represented in FIGS. 24a-24d, or a variant amino acid sequence wherein said variant is the deletion, substitution or addition of at least one amino acid residue represented in FIGS. 24a-24d.

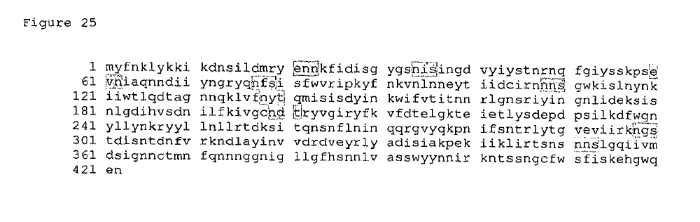

In a preferred embodiment of the invention said recombinant, represented in FIG. 25, or antigenic part thereof, or a variant amino acid sequence wherein said variant is the deletion, substitution or addition of at least one amino acid residue represented in FIG. 25.

In a preferred embodiment of the invention said microbial cell is a bacterial cell.

Glycoconjugate vaccines according to the invention can be prepared by two methods:
i. Expression of the oligosaccharyltransferase polypeptide and the modified glycoprotein acceptor in the host organism expressing the polysaccharide to be conjugated. This may require using a genetically modified host, e.g. an O-antigen ligase mutant.
ii. Expression of the oligosaccharyltransferase polypeptide the modified glycoprotein and the cloned polysaccharide biosynthesis locus in an E. coli host.

The microbial cell will preferably of the genus Escherichia, for example E. coli; alternatively said bacterial cell is of the genus Salmonella spp.

According to an aspect of the invention there is provided a vaccine composition comprising a bacterial glycoconjugate antigen polypeptide, or part thereof, according to the invention.

In a preferred embodiment of the invention said composition includes a carrier and/or optionally an adjuvant.

In a preferred embodiment of the invention said adjuvant is selected from the group consisting of: cytokines selected from the group consisting of GMCSF, interferon gamma, interferon alpha, interferon beta, interleukin 12, interleukin 18, interleukin 23, interleukin 17, interleukin 2, interleukin 1, TGF, TNFα, and TNFβ.

In a further alternative embodiment of the invention said adjuvant is a TLR agonist such as CpG oligonucleotides, flagellin, monophosphoryl lipid A, poly I:C and derivatives thereof.

In a preferred embodiment of the invention said adjuvant is a bacterial cell wall derivative such as muramyl dipeptide (MDP) and/or trehelose dycorynemycolate (TDM).

An adjuvant is a substance or procedure which augments specific immune responses to antigens by modulating the activity of immune cells. Examples of adjuvants include, by example only, agonistic antibodies to co-stimulatory molecules, Freunds adjuvant, muramyl dipeptides, and liposomes. An adjuvant is therefore an immunomodulator. A carrier is an immunogenic molecule which, when bound to a second molecule augments immune responses to the latter.

In a preferred embodiment of the invention said composition comprises a mix of two or three different glycoconjugate antigenic polypeptides as herein described.

According to a further aspect of the invention there is provided a cell culture comprising a microbial cell according to the invention.

According to a further aspect of the invention there is provided a fermentor comprising a microbial cell culture according to the invention.

According to a further aspect of the invention there is provided the use of a cell according to the invention in the production of glycoconjugated polypeptides.

According to an aspect of the invention there is provided a method for the production of a recombinant glycoconjugate polypeptide comprising:
i) providing a microbial culture according to the invention;
ii) culturing the microbial culture; and
iii) isolating the glycoconjugate polypeptide from the micobial cells or the cell culture medium.

Microbial cells used in the process according to the invention are grown or cultured in the manner with which the skilled worker is familiar, depending on the host organism. As a rule, microbial cells are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen.

The pH of the liquid medium can either be kept constant, that is to say regulated during the culturing period, or not. The cultures can be grown batchwise, semi-batchwise or continuously. Nutrients can be provided at the beginning of the fermentation or fed in semi-continuously or continuously. The products produced can be isolated from the organisms as described above by processes known to the skilled worker. To this end, the organisms can advantageously be disrupted beforehand. In this process, the pH value is advantageously kept between pH 4 and 12, preferably between pH 6 and 9, especially preferably between pH 7 and 8.

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogenphosphate or dipotassium hydrogenphosphate or the corresponding sodium-containing salts may be used as sources of phosphorus.

Chelating agents may be added to the medium in order to keep the metal ions in solution. Particularly suitable chelating agents comprise dihydroxyphenols such as catechol or protocatechuate and organic acids such as citric acid.

The fermentation media used according to the invention for culturing microbial cells usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

The culture temperature is normally between 15° C. and 45° C., preferably at from 25° C. to 40° C., and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of plasmids it is possible to add to the medium suitable substances having a selective effect, for example antibiotics. Aerobic conditions are maintained by introducing oxygen or oxygen-containing gas mixtures such as, for example, ambient air into the culture. The culture is continued until formation of the desired product is at a maximum. This aim is normally achieved within 10 to 160 hours.

According to a further aspect of the invention there is provided a method to vaccinate a subject to a bacterial infection comprising immunizing said subject with an effective amount of a vaccine according to the invention.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1a is the nucleotide sequence of a Nitratiruptor tergasus pglB orthologue; FIG. 1b is the amino acid sequence of Nitratiruptor tergasus PglB orthologue;

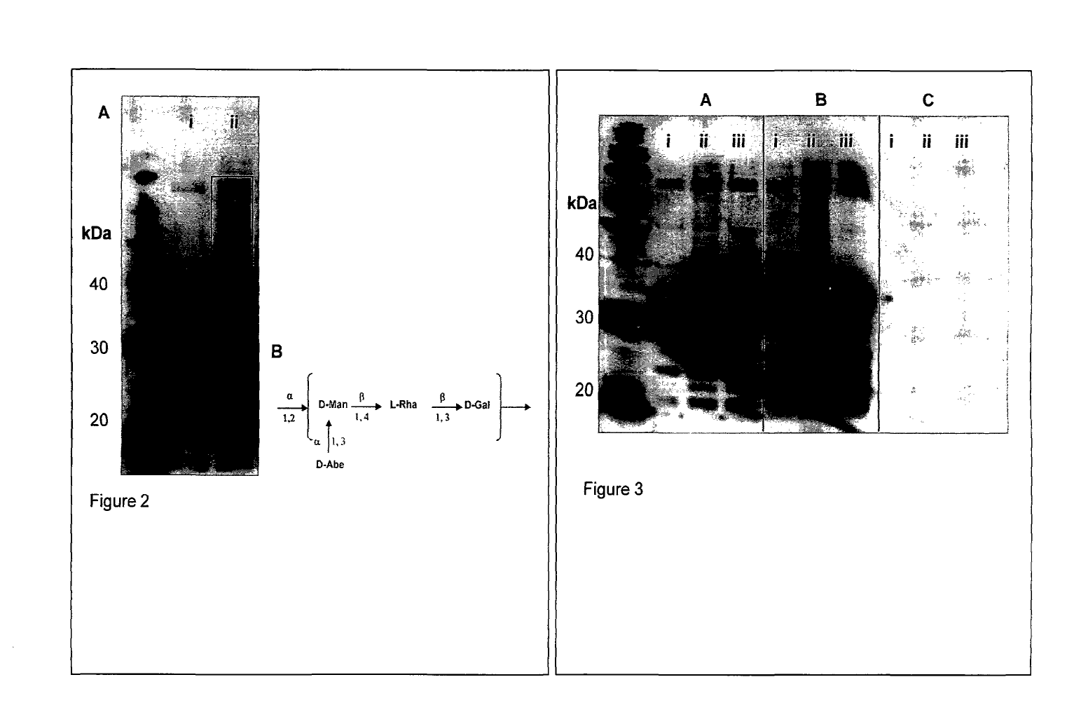

FIG. 2 A) Detection of CJ0114-His. Recombinant CJ0114-His co-expressed in S. Typhimurium with i) pMAF10 [C. jejuni PglB (Cj PglB)] and ii) pMLNT2 [N. tergacus PglB (Nt PglB)], detected with anti-His antibody. Box indicates ladder pattern reminiscent of O-antigen. B) Structure of S. Typhimurium O-antigen. Galactose at the reducing end is not permissive for transfer to protein by Cj PglB; and

FIG. 3 Proteinase K treatment of purified CJ0114-His. Purified CJ0114-His from S. Typhimurium (i), S. Typhimurium co-expressing Nt PglB (ii) and S. Typhimurium co-expressing Cj PglB (iii), A) 2.5 μg CJ0114-His, B) 2.5 μg CJ0114-His following incubation at 37° C. for 16 h, C) 2.5 μg CJ0114-His following incubation with Proteinase K at 37° C. for 16 h. CJ0114-His was detected with anti-His antibody. The absence of reactivity to the His antibody following Proteinase K treatment indicates that the ladder-like pattern identified is of protein origin;

FIG. 4a is the amino acid sequence of Streptococcus pneumoniae pneumolysin; FIG. 4b is the amino acid sequence a non-toxic variant pnuemolysin; FIG. 4c is the amino acid sequence of Streptococcus pneumoniae PspA; FIG. 4d is the amino acid sequence of Streptococcus pneumoniae unknown antigen; and FIG. 4e is the amino acid sequence of Streptococcus pneumoniae ABC transporter, substrate binding protein;

FIG. 5 is the amino acid sequence of Corynebacterium diphtheriae toxin CRM197;

FIG. 6a is the amino acid sequence of Streptococcus suis antigen; FIG. 6b is the amino acid sequence of Streptococcus suis surface antigen SP1 antigen; FIG. 6c is the amino acid sequence of Streptococcus suis Rfe A antigen; FIG. 6d is the amino acid sequence of Streptococcus suis unknown antigen; FIG. 6e is the amino acid sequence of Streptococcus suis dehydrogenase antigen; FIG. 6f is the amino acid sequence of Streptococcus suis hemolysin; and FIG. 6g is the amino acid sequence of Streptococcus suis phosphoglycerate mutase

FIG. 7a is the amino acid sequence of Streptococcus agalactiae C5a peptidase; FIG. 7b is the amino acid sequence of Streptococcus agalactiae immunoglobulin A binding beta antigen; FIG. 7c is the amino acid sequence of Streptococcus agalactiae metal binding protein AcdA; FIG. 7d is the amino acid sequence of Streptococcus agalactiae LysM antigen; FIG. 7e is the amino acid sequence of Streptococcus agalactiae LPXTG antigen; FIG. 7f is the amino acid sequence of the Streptococcus agalactiae cell wall surface anchor family protein; FIG. 7g is the amino acid sequence of the Streptococcus agalactiae cell wall surface anchor family protein; FIG. 7h is the amino acid sequence of the Streptococcus agalactiae cell wall surface anchor family protein; FIG. 7i is the amino acid sequence of the Streptococcus agalactiae cell Bib A antigen; FIG. 7j is the amino acid sequence of the Streptococcus agalactiae C protein immunoglobulin-A-binding beta antigen; FIG. 7k is the amino acid sequence of the Streptococcus agalactiae surface protein Rib antigen; FIG. 7l is the amino acid sequence of the Streptococcus agalactiae alpha like protein 3; and FIG. 7m is the amino acid sequence of the Streptococcus agalactiae alpha like protein 2;

FIG. 8a is the amino acid sequence of Klebsiella pneumoniae Fim A antigen; FIG. 8b is the amino acid sequence of Klebsiella pneumoniae putative fimbriae major subunit; FIG. 8c is the amino acid sequence of Klebsiella pneumoniae MrkA antigen; FIG. 8d is the amino acid sequence of Klebsiella pneumoniae OmpA; FIG. 8e is the amino acid sequence of Klebsiella pneumoniae MrkD; FIG. 8f is the amino acid sequence of Klebsiella pneumoniae FepA; FIG. 8g is the amino acid sequence of Klebsiella pneumoniae OmpK36; FIG. 8h is the amino acid sequence of Klebsiella pneumoniae OmpK17; and FIG. 8i is the amino acid sequence of Klebsiella pneumoniae OmpW;

FIG. 9a is the amino acid sequence of Streptococcus iniae Sim A antigen; FIG. 9b is the amino acid sequence of Streptococcus iniae Scpl antigen;

FIG. 10a is the amino acid sequence of HPV coat protein L1; FIG. 10b is the amino acid sequence of HPV major capsid protein L1;

FIG. 11a is the amino acid sequence of Pseudomoas aeruinosa OmpA; FIG. 11b is the amino acid sequence of Pseudomoas aeruinosa OprF; FIG. 11c is the amino acid sequence of Pseudomoas aeruinosa Opr I; FIG. 11d is the amino acid sequence of Pseudomoas aeruinosa Fli C; FIG. 11e is the amino acid sequence of Pseudomoas aeruinosa KatE; FIG. 11f is the amino acid sequence of Pseudomoas aeruinosa Kat A; FIG. 11g is the amino acid sequence of Pseudomoas aeruinosa amidase; FIG. 11h is the amino acid sequence of Pseudomoas aeruinosa Opr86; FIG. 11i is the amino acid sequence of Pseudomoas aeruinosa LcrV; FIG. 11j is the amino acid sequence of Pseudomoas aeruinosa Tox A; and FIG. 11k is the amino acid sequence of Pseudomoas aeruinosa exotoxin;

FIG. 12a is the amino acid sequence of Actinebacter baumanni FhuE;

FIG. 13a is the amino acid sequence of Salmonella enterica OmpD; FIG. 13b is the amino acid sequence of Salmonella enterica SopB; FIG. 13c is the amino acid sequence of Salmonella enterica GroEL; FIG. 13d is the amino acid sequence of Salmonella enterica PagC; FIG. 13e is the amino acid sequence of the Salmonella enterica fimbrial subunit; FIG. 13f is the amino acid sequence of Salmonella enterica DnaJ; FIG. 13g is the amino acid sequence of Salmonella enterica OmpC; FIG. 13h is the amino acid sequence of Salmonella enterica OmpF FIG. 131 is the amino acid sequence of a Salmonella enterica outer membrane protein;

FIG. 14 is the amino acid sequence of Bacillus anthracis protective antigen (PagA);

FIG. 15a is the amino acid sequence of Campylobacter jejuni Peb 1; FIG. 15b is the amino acid sequence of Campylobacter jejuni Peb 2; FIG. 15c is the amino acid sequence of Campylobacter jejuni Peb 3; FIG. 15d is the amino acid sequence of Campylobacter jejuni Cj0114; FIG. 15e is the amino acid sequence of Campylobacter jejuni Cja A; and FIG. 15f is the amino acid sequence of Campylobacter jejuni FlaA;

FIG. 16a is the amino acid sequence of Haemophilus influenza protein D; FIG. 16b is the amino acid sequence of Haemophilus influenza Hap; FIG. 16c is the amino acid sequence of Haemophilus influenza PiIA; FIG. 16d is the amino acid sequence of Haemophilus influenza Omp P5; FIG. 16e is the amino acid sequence of Haemophilus influenza Hia; and FIG. 16f is the amino acid sequence of Haemophilus influenza HMW1;

FIG. 17 is the amino acid sequence of Bordetella pertussis pertactin;

FIG. 18 is the amino acid sequence of Escherichia coli antigen 43

FIG. 19 is the amino acid sequence of Helicobacter pylori UreB;

FIG. 20a is the amino acid sequence of Neisseria meningitidis NadA; FIG. 20b is the amino acid sequence of Neisseria meningitidis GNA1870; FIG. 20c is the amino acid sequence of Neisseria meningitidis Fet A;

FIG. 21a is the amino acid sequence of Plasmodium falciparum merozoite surface protein 4 MSP4; FIG. 20b is the amino acid sequence of Plasmodium falciparum merozoite surface protein 5; MSP5

FIG. 22a is the amino acid sequence of Staphylococcus aureus ClfA; FIG. 22b is the amino acid sequence of Staphylococcus aureus enolase; FIG. 22c is the amino acid sequence of Staphylococcus aureus 3-oxoacyl reductase; FIG. 22d is the amino acid sequence of Staphylococcus aureus hypothetical protein SAS2241; FIG. 22e is the amino acid sequence of Staphylococcus aureus IsdB; FIG. 22f is the amino acid sequence of Staphylococcus aureus exotoxin;

FIG. 23a is the amino acid sequence of Clostridium difficile SLP; FIG. 23b is the amino acid sequence of Clostridium difficile FliC; FIG. 23c is the amino acid sequence of Clostridium difficile FliD; FIG. 23d is the amino acid sequence of Clostridium difficile Cwp84; FIG. 23e is the amino acid sequence of Clostridium difficile Cwp66; FIG. 23f is the amino acid sequence of Clostridium difficile Toxin A;

FIG. 24a is the amino acid sequence of Mycobacterium tuberculosis Cfp-10; FIG. 24b is the amino acid sequence of Mycobacterium tuberculosis Ag85A; FIG. 24c is the amino acid sequence of Mycobacterium tuberculosis Ag85B; FIG. 24d is the amino acid sequence of Mycobacterium tuberculosis ESAT-6;

FIG. 25 is the amino acid sequence of Recombinant botulinum Toxin F Hc domain [synthetic construct];

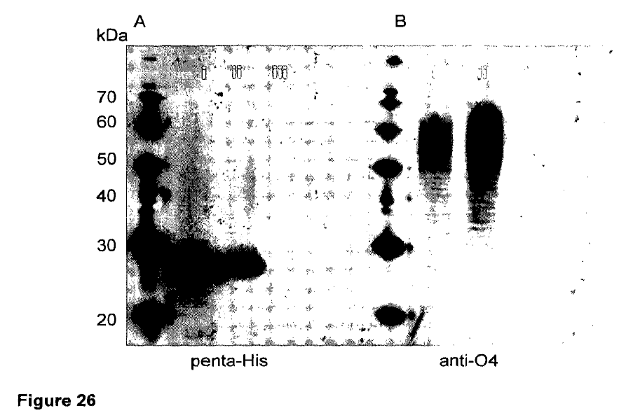

FIG. 26 Illustrates proteinase K treatment of purified CJ0114-His. i) 2.5 μg protein ii) 2.5 μg protein following incubation at 37° C. for 16 h C, iii) 2.5 μg protein following incubation with Proteinase K at 37° C. for 16 h;

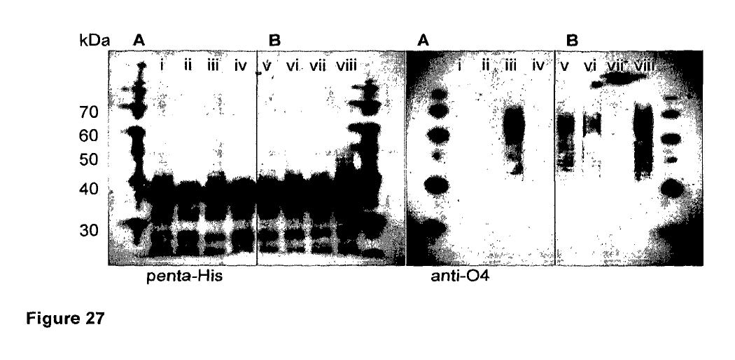

FIG. 27 Identification of catalytic motifs. CJ0114-His purified from S. Typhimurium SL3749 was detected in Western blot with penta-His and anti-O4 antibodies. A) Mutagenesis of Nt PglB, i) CJ0114-His alone, ii) CJ0114-His expressed with Cj PglB, iii) CJ0114-His expressed with Nt PglB, iv) CJ0114-His expressed with Nt PglB_{WAAYG} B) Mutagenesis of CJ0114-His. v) CJ0114-His_{N101Q}, vi)CJ0114-His_{N155Q}, vii)CJ0114-HiS_{N173Q}, viii)CJ0114-His_{N179Q}, and

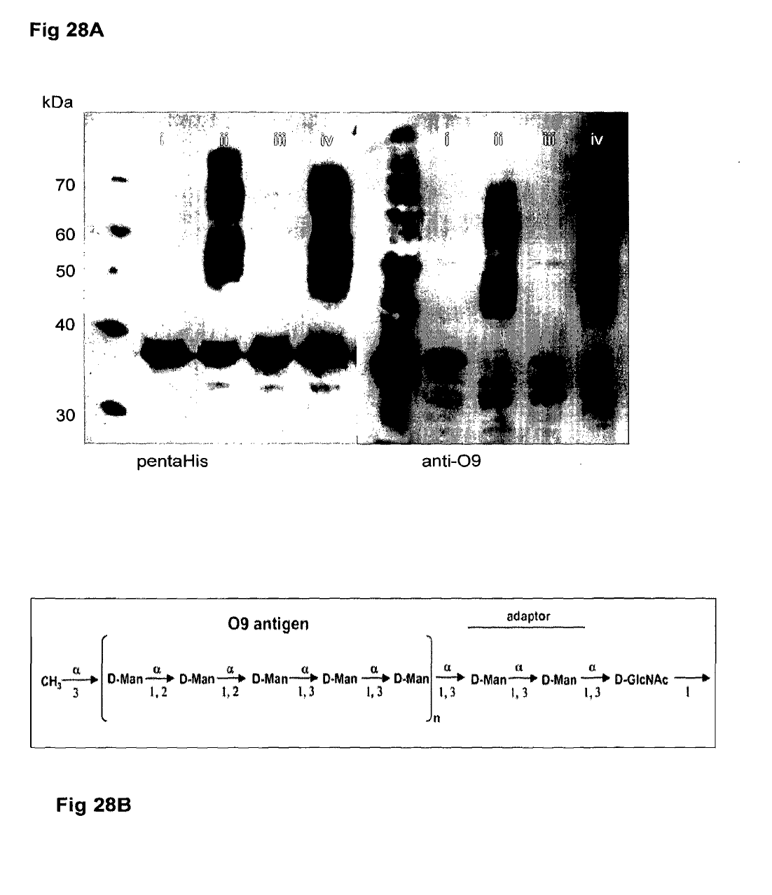

FIG. 28 A) Western immunoblot to detect transfer of O9 O-antigen to CJ0114-His with anti-His antibody and anti-O9. CJ0114-His was expressed in E. coli E69 (lanes i and iii) and in the same strain with Cj PglB (lane ii) or Nt PglB (lane iv). In the presence of either oligosaccharyltransferase, O9 is detected by both the protein-specific antibody (anti-His) and the O9 specific antibody. B) Structure of E. coli O9.

### MATERIALS AND METHODS

### Bacterial Strains, Plasmids and Genomic DNA

E. coli and Salmonella enterica sv. Typhimurium strains were grown on LB at 37° C. Where appropriate, 50 μg/ml trimethoprim and/or 100 μg/ml ampicillin were added to the media. E. coli DH5α (Invitrogen) and XL-1 (Stratagene) were used as hosts for cloning experiments. S. Typhimurium strain SL3749 was obtained from the Salmonella Genetic Stock Centre (SGSC). Genomic DNA from Nitratiruptor tergacus SB155-2 was kindly supplied by Satoshi Nakagawa at the Japan Agency for Marine-Earth Science and Technology (JAMSTEC). Plasmids pMLBAD (1) and pETBlue-1 (Novagen) were used as cloning vectors.

### Construction of Plasmids

The N. tergacus pglB orthologue was amplified from genomic DNA with primers Nit pglB-F (5-AGGAATTCAGATGTATGTGCAAAAAAAG-3, EcoRI site underlined) and Nit pglB-HA-R (ACAAACTAGTTTAAGCGTAATCTGGAACATCGTATGGGTATTTGATTCTATAAATTT TCA-3, SpeI site underlined, HA-encoded sequence in bold) with Pfx polymerase (Invitrogen). The resulting amplicon was digested with EcoRI and SpeI and ligated to EcoRI/XbaI-digested pMLBAD, generating pMLNT2. C. jejuni Cj0114 was amplified from C. jejuni 11168H genomic DNA with primers Cj0114-F (5-ATGAAAAAAATATTCACAGTAGCTC-3) and Cj0114-R (5-TTAGTGATGGTGATGGTGATGTTTTCTATTAGGTGAAGCTTTTG-3, 6×H-encoded sequence in bold) with Pfx polymerase and was then blunt-end cloned in EcoRV-digested pETBlue-1. All vectors were confirmed by restriction analysis and sequencing.

### Protein Expression

To check for expression of PglB_{NT}-HA, E. coli transformed with pMLNT2 was grown to mid-log phase and protein expression was induced with 0.1% arabinose for 4 hours. Whole cells were suspended in 1×SDS-PAGE loading buffer and heated to 60° C. for 20 minutes. The lysates were resolved on 10% Tris-glycine polyacrylamide gels (Invitrogen), transferred to nitrocellulose and probed with anti-HA-HRP antibody (Roche). For expression of Cj0114-His protein, pET[CJ0114] was transformed in E. coli, which were grown to mid-log phase and protein expression was induced with 1 mM IPTG for 4 hours. CJ0114 was detected in whole cell lysates with a Penta-His Ab (QIAgen).

### Expression and Purification of Proteins in S. Typhimurium

S. Typhimurium SL3749 was transformed with pMLNT2 and pET[CJ0114] and 200 ml was grown to mid-log phase (A₆₀₀˜0.5). Protein expression was induced in 200 ml cultures with 0.1% arabinose and 1 mM IPTG for 16 h at 37° C. Following centrifugation the bacterial pellet was lysed with 1× BugBuster (Novagen) in Lysis buffer (50 mM NaH₂PO₄, 300 mM NaCl, 10 mM imidazole) supplemented with 1 mg/ml Lysozyme (Sigma), 1 μl/ml Benzonase nuclease (Novagen) and 0.1% Tween-20. The cleared lysate was then incubated with 1 ml Ni-NTA agarose slurry (QIAgen) with stirring at 4° C. and subsequently loaded into an empty 5 ml polypropylene column (Pierce). This was washed 5 times with 1 column volume wash buffer (50 mM NaH₂PO₄, 300 mM NaCl, 20 mM imidazole supplemented with 0.1% Tween 20) and CJ0114-His was then eluted 4 times with 500 μl Elution buffer (50 mM NaH₂PO₄, 300 mM NaCl, 250 mM imidazole supplemented with 0.1% Tween-20). Following confirmation of purification by Western blot analysis, eluants were pooled and protein was concentrated 10-fold with Amicon Ultra-4 Centrifugal Filter Unit with Ultracel-10 membrane (Millipore). Protein was quantified by BCA assay (Pierce).

### Proteinase K Treatment

2.5 μg CJ0114-His was incubated with 200 μg Proteinase K and 5 mM CaCl at 37° C. for 16 hours. Control reactions were incubated with water in place of Proteinase K.

### Western Immunoblot Detection of CJ0114-His

Samples of CJ0114-His were resolved on 12% Tris-glycine gels (Invitrogen) and transferred to nitrocelluose membrane. Membranes were blocked in His blocking reagent (QIAgen) for 1 hour at room temperature and then probed with anti-His-HRP (1:5000 in blocking reagent, QIAgen) for 1 hour at room temperature. The membranes were then washed twice with PBS-T/T (phosphate buffered saline supplemented with 0.05% Tween and 0.5% Triton) and once with PBS before His-tagged proteins were detected with Amersham ECL Plus (GE Healthcare).

(1) Lefebro and Valvano (2002) Construction and Evaluation of Plasmid Vectors Optimized for Constitutive and Regulated Gene Expression in Burkholderia cepacia Complex Isolates, APPLIED AND ENVIRONMENTAL MICROBIOLOGY 68(12), 5956-5964.

All polysaccharides listed could potentially be conjugated to CRM₁₉₇ (the carrier protein for the currently licensed S. pnemoniae conjugate vaccine as well as other glycoconjugates). This is a non-toxic form of Diptheria toxin and has a naturally occurring glycosylation motif (442-DVNKS-446; see FIG. 5, motif highlighted). Details of bacteria-specific antigens are provided for each polysaccharide. These could potentially provide enhanced immunity for the given organism (in other words the best of both worlds protein and polysaccharide induced immunity for long term full coverage). It is also feasible to combine the polysaccharide(s) from one organism with an immunogenic protein from a different organism to create a dual vaccine (or if this was performed on a rationally attenuated vaccine delivery strain, eg Salmonlella, a triple vaccine). (examples of triple vaccines are a travelers diarrhea vaccine, consisting of Shigella sonnei O antigen coupled to cholera toxin in a Salmonella or EPEC attenuated carrier strain).

Strategies for Expression of Heterologous Polysaccharides in E. coli
1 Where sequence data is available for the polysaccharide biosynthesis locus, long-range high-fidelity polymerase chain reaction (PCR) will be used to amplify the fragment from genomic DNA. The resulting amplicon will be cloned in a low-copy plasmid vector, such as pACYC184 or pBR322, using conventional cloning techniques. Successful expression of polysaccharide in the E. coli host (or alternative bacterial host) will be confirmed by Western blot analysis with antibodies specific to the polysaccharide of interest (where available).
2 For the expression of polysaccharides for which the genetic locus has not been confirmed, or where PCR amplification is unsuitable, cosmid libraries will be generated from genomic DNA and screened to identify clones comprising the genetic information required for polysaccharide biosynthesis.

### Cloning of Glycoacceptor Proteins

The polypeptide acceptor substrates will be amplified by PCR using High Fidelity polymerase and cloned in an expression vector, i.e. pETBlue. A 6× Histadine tag will be incorporated at the 3′ end of the ORF to facilitate protein purification. D/E-X-N-S/T motifs will be engineered into the cloned ORFs by site directed mutagenesis.

### General Method for Production of Recombinant Glycoconjugate Polypeptides

The preferential host for production of recombinant glycoconjugate polypeptides will be Escherichia coli. This will be transformed with three plasmids:
i. Low-copy plasmid or cosmid encoding the polysaccharide biosynthesis locus.
ii. An expression plasmid encoding the glycoacceptor protein comprising at least one D/E-X-N-S/T motif.
iii. pMLNT2 (encoding N. tergacus PglB).

Recombinant E. coli will be cultured initially in volumes of 200 ml-1 L of LB broth, supplemented with selective antibiotics where appropriate. Protein expression will be induced at mid-log phase of growth and cells will be harvested following 4 h-24 h expression at 16-37 C (specific conditions to be optomised for each glycoconjugate). Recombinant glycoconjugate will be purified using method mentioned for purification of CJ0114-His from S. Typhimurium. Glycosylation will be confirmed by Western blot analysis with ant-His and specific anti-polysaccharide antibodies.

Where alternative bacterial hosts are used for the expression of glycoconjugates the same protocol will be followed but culture conditions may be changed to those optimal for the given host.

### EXAMPLE 1

### Selected Antigens

Streptococcus pneumoniae capsule from serotypes with glucose or galactose at the reducing end (i.e. 1, 2, 3, 6A, 6B, 7F, 7A, 7B, 8, 9A, 9L, 9N, 9V, 10F, 10A, 11F, 11A, 11B, 11C, 13, 14, 15F, 15A, 15B, 15C, 17F, 17A, 18F, 18A, 18B, 18C, 19F, 19A, 19B, 19C, 22F, 23F, 27F, 29, 31, 32F, 32A, 33F, 33B, 34, 35A, 35B, 37) S. pneumoniae protein antigens to which these could be coupled include a non-toxic variant of the S. pneumoniae Pnemolysin, where the Tryptophan at 433 is mutated to Phenylyalanine. This has previously been shown to induce immunity in mice and has been proposed as a candidate for human vaccination [1]. The sequence of this protein includes five N-X-S/T sites that could potentially be modified to the acceptor motif D/E-X-N-Y-S/T (see attachment 3, motifs highlighted). Additional S. pneumoniae immunogens include PspA, PspC and PsaA [2,3] (see attachment 3, potential partial motifs are highlighted).

Streptococcus suis capsule. Although the structure has not been determined, the capsule region has been identified in the genome. S. suis immunogens include Enolase, Sao, HP0197, RfeA, ESA, IBP, SLY and a 38-kDa protein [4,5,6,7,8].

Group B Streptococcus (Streptococcus agalatiae): All nine human serotypes (Ia, Ib, II, III, IV, V, VI, VII, VIII) have glucose at the reducing end. GBS protein antigens include ScpB, β-component of the C protein, LmbP, Sip, LrrG, SAG1408, SAG0645, SAG0649, BibA and the Alp family of proteins (α, Rib, R28 and Alp2) [9,10,11] (see attachment 5). A suggested vaccine strategy for GBS is the immunization of non-pregnant adolescents, and could therefore potentially be linked to HPV capsid proteins to provide a dual vaccine (see attachment 6 for amino acid sequences of capsid proteins from HSV types currently used for GSK vaccine in the UK). There is also the possibility of developing vaccine for strains that colonize mammary glands of ruminants, affecting milk quality and quantity (could be linked to antigens from other bacteria that also cause mastitis in dairy, e.g. E. coli, Staph aureus, Strep. uberis and Strep. dysgalactiae

Streptococcus iniae (fish pathogen)—of global veterinary importance and there is need for vaccine. No structural data on capsule but genes show some similarity to S. agalatiae. Potential protein antigens include M-like proteins SimA and Spcl [12,13].

Klebsiella spp. 77 capsule serotypes and 7 O antigen many serotypes K. Protein immunogens for Klebsiella include major structural proteins of type 1 and type 3 fimbriae, Outer membrane proteins OmpA, OmpW, OmpK17, OmpK36 and FepA.

Pseudomonas aeruginosa: Produces A- and B-band O-antigen. A band has rhamnose at reducing end, 10 serotypes for B-band, most FucNAc, three Rha, 1 Rib. Also produces an exoplysaccharide with Man at the reducing end. P. aeruginosa protein antigens include toxins and outer membrane proteins).

### EXAMPLE 2

### Confirmation of Specific Transfer of S. Typhimurium O4 Using O4-Specific Antisera (Mouse Monoclonal [1E6], ab8274, Abcam UK)

CJ0114-His and Nt PglB were expressed in S. Typhimurium SL3749 (waaL-). CJ0114-His was purified by Ni-NTA affinity under denaturing conditions (8M urea) and purified samples were resolved by SDS-PAGE, transferred to nitrocellulose and probed with anti-H is or anti-O4 antibodies. S. Typhimuirium O4 was detected as a polymeric ladder-like structure at molecular mass greater than the unmodified CJ0114-His protein, FIG. 26B. To confirm that O-antigen was attached to protein, samples were treated with Proteinase K. No O4-reactive species were identified in the treated sample, indicating that the O-antigen is attached to protein.

### EXAMPLE 3

### Identification of Active Sites and Confirmation of N-Linked Transfer

a. The essential oligosaccharyltransferase motif (WWDYG) was mutated in Nt PglB to WAAYG by site-directed mutagenesis. Either the wild-type or the mutated Nt PglB enzyme was expressed with CJ0114-His in S. Typhimurium SL3749 and CJ0114-His was subsequently purified under denaturing conditions and detected by Western blot with anti-His and anti-O4. S. Typhimurium O4 was only detected when CJ0114-His was co-expressed with wild-type Nt PglB, FIG. 27. The ₄₆₈WAAYG₄₇₂ mutant was unable to transfer O-antigen to protein, indicating that Nt PglB is functioning specifically as an N-linked oligosaccharyltransferase.

b. To further investigate the specificity of Nt PglB, each of the four D/E-X-N-X-S/T acceptor sequons in the CJ0114-His acceptor protein (at amino acid position 101, 155, 173 and 179) were mutated to D/E-X-Q-X-S/T. The mutated CJ0114-His proteins were expressed with Nt PglB in S. Typhimurium SL3749 and transfer of O4 O-antigen was detected by Western blot with anti-His and anti-O4 antibodies, Transfer of O antigen to protein was detected in three of the mutated acceptor proteins (N101Q, N155Q and N179Q) but was abolished when CJ0114-His_{N179Q} was used as the acceptor protein, indicating that only the ₁₇₁DSNST₁₇₅ sequon is used by Nt PglB. This also confirms that the sugar is attached to the protein on an asparagine residue (i.e. specifically N-linked transfer).

### EXAMPLE 4

In addition to S. Typhimurium O4, Nt PglB is able to transfer E. coli O9 (see FIG. 28A). Nt PglB or Cj PglB were expressed with CJ0114-His in E. coli E69 (O9K30) and CJ0114-His was subsequently purified. Transfer of O9 to CJ0114-His was confirmed by Western immunoblot with both anti-His antibody and anti-O9. The reducing end sugar of the O9 O-antigen in this strain is N-acetylglucosamine, which has previously been shown to be a substrate for Cj PglB. This result indicates that there are similarities, as well as differences in the specificity of these two oligosaccharyltransferases.

### REFERENCES

[1] Alexander et al. (1994) Immunization of Mice with Pneumolysin Toxoid Confers a Significant Degree of Protection against At Least Nine Serotypes of Streptococcus pneumoniae. Infect. Immun. 62, 5683-5688.
[2] Meng et al (2009) Development of a 5-valent conjugate pneumococcal protein A-capsular polysaccharide pneumococcal vaccine against invasive pneumococcal disease. Microbial Pathogenesis 47, 151-156.
[3] Xin et al. (2009) PspA family fusion proteins delivered by attenuated Salmonella enterica serovar Typhimurium extends and enhances protection against Streptococcus pneumoniae. Infect. Immun. (published online).
[4] Zhang et al., (2009) Identification and characterization of a novel protective antigen, Enolase of Streptococcus suis serotype 2. Vaccine 27, 1348-1353.
[5] Li et al. (2006) Ideintificatino of a surface protein of Streptococcus suis and evaluation of its immunogenic and protective capacity in pigs. Infect. Immun. 74, 305-312.
[6] Zhang et al. (2009) Identification of a surface protective antigen, HP0197 of Streptococcus suis serotype 2. Vaccine 27, 5209-5213.
[7] Liu et al. (2009) Identification and experimental verification of protective antigens against Streptococcus suis serotype 2 based on genome sequence analysis. Curr. Microbiol. 58, 11-17.
[8] Okwumabua et al, (2005) Identification of the gene encoding a 38-Kilodalton immunogenic and protective antigen of Streptococcus suis. Clinical and Diagnostic Lab. Immunol. 12, 484-490.
[9] Johri et al. (2006) Group B Streptococcus: global incidence and vaccine development. Nature Reviews Microbiology 4, 932-942.
[10] Santi et al. (2009) BibA induces opsonizing antibodies conferring in vivo protection against Group B Streptococcus. J. Infect. Dis. 200, 564-570.
[11] Lindahl et al. (2005) Surface proteins of streptococcus agalactiae and related proteins in other bacterial pathogens. Clin. Microbiol. Rev. 18, 102-127.
[12] Locke et al. (2008) Streptococcus iniae M-Like Protein Contributes to Virulence in Fish and Is a Target for Live Attenuated Vaccine Development. PLoS One 3, e2824.
[13] Baiano et al. (2008) Identification and molecular characterisation of a fibrinogen binding protein from Streptococcus iniae. BMC Microbiology 8
[14] Witkowska et al. (2005) Major structural proteins of type 1 and type 3 Klebsiella fimbriae are effective protein carriers and immunogens in conjugates as revealed from their immunochemical characterization. FEMS Immunol. And Med. Micro. 45, 221-230.

1. A microbial cell transformed with a vector comprising a nucleotide sequence selected from the group consisting of
i) a nucleic acid molecule consisting of a nucleic acid sequence as represented in FIG. 1a;
ii) a nucleic acid molecule consisting of a nucleic acid sequence that hybridises under stringent hybridisation conditions to the nucleic acid molecule in (i) and which encodes an polypeptide; wherein said cell expresses a recombinant polypeptide which is a substrate for said oligosaccharyltransferase polypeptide.

2. A microbial cell according to claim 1 wherein said cell is transformed with a nucleic acid molecule comprising a nucleotide sequence that encodes an oligosaccharyltransferase polypeptide as represented by the amino acid sequence in FIG. 1b, or a variant polypeptide and comprises the amino acid sequence represented in FIG. 1b which sequence has been modified by deletion, addition or substitution of at least one amino acid residue and which retains or has enhanced oligosaccharyltransferase activity.

3. A microbial cell according to claim 1 or 2 wherein said vector is an expression vector adapted for expression of a nucleic acid molecule encoding the oligosaccharyltransferase polypeptide.

4. A microbial cell according to any of claims 1-3 wherein said recombinant polypeptide includes at least one peptide motif consisting of the amino acid sequence:
Asp/Glu-Xaa₁-Asn-Xaa₂-Ser/Thr
wherein Xaa₁ and Xaa₂ is any amino acid except proline.

5. A microbial cell according to any of claims 1-4 wherein said recombinant polypeptide is an antigen isolated from an infectious agent.

6. A microbial cell according to claim 5 wherein said infectious agent is a bacterial pathogen.

7. A microbial cell according to claim 5 or 6 wherein said agent is selected from the group consisting of: Streptococcus spp, Streptococcus pneumoniae, Streptococcus suis, Streptococcus inae, Streptococcus agalactiae, Streptococcus dysgalactiae, Streptococcus equi, Streptococcus uberist, Streptococcus milleri group (SMG), Streptococcus sanguis, Streptococcus bovis, Streptococcus group A, Klebsiella pneumoniae, Klebsiella oxytoca, Klebsiella planticola, Pseudomonas aerunginosa, Acinetobcater baumanii, Acinetobacter calcoaceticus, Salmonella enterica serovar Typhi, Campylobacter jejuni, Campylobacter coli, Campylobacter lari, Haemophilus influenzae, Helicobacter pylori, Neisseria meningitidis, Neisseria gonorrhoea, Neisseria lactamica, Shigella spp., Staphylococcus spp, Clostridium difficile, Clostridium botulinum, Bacillus anthracis, Lactobacillus spp., Clostridium tetani, Corynebacterium diptheriae, Vibrio cholerae, Escherichia coli, Mycobacterium tuberculosis, Human papillomavirus (HPV), Hepatitis A, Hepatitis B, Rotavirus, virally derived antigens, for example Influenza virus, antigen derived from parasites, for example, Plasmodium faciparum.

8. A microbial cell according to claim 7 wherein said agent is selected from the group consisting of: Staphylococcus epidermidis, S. aureus, S. hominis, S. haemolyticus, S. warneri, S. capitis, S. saccharolyticus, S. auricularis, S. simulans, S. saprophyticus, S. cohnii, S. xylosus, S. cohnii, S. warneri, S. hyicus, S. caprae, S. gallinarum, S. intermedius, S. hominis.

9. A micobial cell according to any of claims 1-8 wherein said oligosaccharyltransferase polypeptide modifies said recombinant polypeptide with a capsular (K) antigen.

10. A micobial cell according to any of claims 1-8 wherein said oligosaccharyltransferase polypeptide modifies said recombinant polypeptide with a capsular O antigen.

11. A microbial cell according to any of claims 1-8 wherein said oligosaccharyltransferase polypeptide modifies said recombinant polypeptide with a exopolysaccharide/surface polysaccharide.

12. A microbial cell according to claim 6 or 7 wherein said recombinant antigenic polypeptide comprises or consists of an amino acid sequence, or antigenic part thereof, selected from the group consisting of the sequences represented in FIGS. 4a-4e, or a variant amino acid sequence wherein said variant is the deletion, substitution or addition of at least one amino acid residue represented in FIGS. 4a-4e.

13. A microbial cell according to claim 6 or 7 wherein said recombinant antigenic polypeptide comprises or consists of an amino acid sequence, or antigenic part thereof, selected from the group consisting of the sequences represented in FIG. 5 or a variant amino acid sequence wherein said variant is the deletion, substitution or addition of at least one amino acid residue represented in FIG. 5.

14. A microbial cell according to claim 6 or 7 wherein said recombinant antigenic polypeptide comprises or consists of an amino acid sequence, or antigenic part thereof, selected from the group consisting of the sequences represented in FIGS. 6a-6g, or a variant amino acid sequence wherein said variant is the deletion, substitution or addition of at least one amino acid residue represented in FIGS. 6a-6g.

15. A microbial cell according to claim 6 or 7 wherein said recombinant antigenic polypeptide comprises or consists of an amino acid sequence, or antigenic part thereof, selected from the group consisting of the sequences represented in FIGS. 7a-7m, or a variant amino acid sequence wherein said variant is the deletion, substitution or addition of at least one amino acid residue represented in FIGS. 7a-7m.

16. A microbial cell according to claim 6 or 7 wherein said recombinant antigenic polypeptide comprises or consists of an amino acid sequence, or antigenic part thereof, selected from the group consisting of the sequences represented in FIGS. 8a-8c, or a variant amino acid sequence wherein said variant is the deletion, substitution or addition of at least one amino acid residue represented in FIGS. 8a-8c.

17. A microbial cell according to claim 6 or 7 wherein said recombinant antigenic polypeptide comprises or consists of an amino acid sequence, or antigenic part thereof, selected from the group consisting of the sequences represented in FIG. 9a or 9b, or a variant amino acid sequence wherein said variant is the deletion, substitution or addition of at least one amino acid residue represented in FIG. 9a or 9b.

18. A microbial cell according to claim 5 wherein said recombinant antigenic polypeptide comprises or consists of an amino acid sequence, or antigenic part thereof, selected from the group consisting of the sequences represented in FIG. 10a or 10b, or a variant amino acid sequence wherein said variant is the deletion, substitution or addition of at least one amino acid residue represented in FIG. 10a or 10b.

19. A microbial cell according to claim 6 or 7 wherein said recombinant antigenic polypeptide comprises or consists of an amino acid sequence, or antigenic part thereof, selected from the group consisting of the sequences represented in FIGS. 11a-11k, or a variant amino acid sequence wherein said variant is the deletion, substitution or addition of at least one amino acid residue represented in FIGS. 11a-11k.

20. A microbial cell according to claim 6 or 7 wherein said recombinant antigenic polypeptide comprises or consists of an amino acid sequence, or antigenic part thereof, represented in FIG. 12, or a variant amino acid sequence wherein said variant is the deletion, substitution or addition of at least one amino acid residue represented in FIG. 12.

21. A microbial cell according to claim 6 or 7 wherein said recombinant antigenic polypeptide comprises or consists of an amino acid sequence, or antigenic part thereof, selected from the group consisting of the sequences represented in FIGS. 13a-13l, or a variant amino acid sequence wherein said variant is the deletion, substitution or addition of at least one amino acid residue represented in FIGS. 13a-13l.

22. A microbial cell according to claim 6 or 7 wherein said recombinant antigenic polypeptide comprises or consists of an amino acid sequence, or antigenic part thereof, represented in FIG. 14, or a variant amino acid sequence wherein said variant is the deletion, substitution or addition of at least one amino acid residue represented in FIG. 14.

23. A microbial cell according to claim 6 or 7 wherein said recombinant antigenic polypeptide comprises or consists of an amino acid sequence, or antigenic part thereof, selected from the group consisting of the sequences represented in FIGS. 15a-15f, or a variant amino acid sequence wherein said variant is the deletion, substitution or addition of at least one amino acid residue represented in FIGS. 15a-15f.

24. A microbial cell according to claim 6 or 7 wherein said recombinant antigenic polypeptide comprises or consists of an amino acid sequence, or antigenic part thereof, selected from the group consisting of the sequences represented in FIGS. 16a-16f, or a variant amino acid sequence wherein said variant is the deletion, substitution or addition of at least one amino acid residue represented in FIGS. 16a-16f.

25. A microbial cell according to claim 6 or 7 wherein said recombinant antigenic polypeptide comprises or consists of an amino acid sequence, or antigenic part thereof, represented in FIG. 17, or a variant amino acid sequence wherein said variant is the deletion, substitution or addition of at least one amino acid residue represented in FIG. 17.

26. A microbial cell according to claim 6 or 7 wherein said recombinant antigenic polypeptide comprises or consists of an amino acid sequence, or antigenic part thereof, represented in FIG. 18, or a variant amino acid sequence wherein said variant is the deletion, substitution or addition of at least one amino acid residue represented in FIG. 18.

27. A microbial cell according to claim 6 or 7 wherein said recombinant antigenic polypeptide comprises or consists of an amino acid sequence, or antigenic part thereof, represented in FIG. 19, or a variant amino acid sequence wherein said variant is the deletion, substitution or addition of at least one amino acid residue represented in FIG. 19.

28. A microbial cell according to claim 6 or 7 wherein said recombinant antigenic polypeptide comprises or consists of an amino acid sequence, or antigenic part thereof, selected from the group consisting of the sequences represented in FIGS. 20a-20c, or a variant amino acid sequence wherein said variant is the deletion, substitution or addition of at least one amino acid residue represented in FIGS. 20a-20c.

29. A microbial cell according to claim 5 wherein said recombinant antigenic polypeptide comprises or consists of an amino acid sequence, or antigenic part thereof, selected from the group consisting of the sequences represented in FIG. 21a or 21b, or a variant amino acid sequence wherein said variant is the deletion, substitution or addition of at least one amino acid residue represented in FIG. 21a or 21b.

30. A microbial cell according to claim 6 or 7 wherein said recombinant antigenic polypeptide comprises or consists of an amino acid sequence, or antigenic part thereof, selected from the group consisting of the sequences represented in FIGS. 22a-22f, or a variant amino acid sequence wherein said variant is the deletion, substitution or addition of at least one amino acid residue represented in FIGS. 22a-22f.

31. A microbial cell according to claim 6 or 7 wherein said recombinant antigenic polypeptide comprises or consists of an amino acid sequence, or antigenic part thereof, selected from the group consisting of the sequences represented in FIGS. 23a-23f, or a variant amino acid sequence wherein said variant is the deletion, substitution or addition of at least one amino acid residue represented in FIGS. 23a-23f.

32. A microbial cell according to claim 6 or 7 wherein said recombinant antigenic polypeptide comprises or consists of an amino acid sequence, or antigenic part thereof, selected from the group consisting of the sequences represented in FIGS. 24a-24d, or a variant amino acid sequence wherein said variant is the deletion, substitution or addition of at least one amino acid residue represented in FIGS. 24a-24d.

33. A microbial cell according to claim 6 or 7 wherein said recombinant antigenic polypeptide comprises or consists of the amino acid sequence represented in FIG. 25, or antigenic part thereof, or a variant amino acid sequence wherein said variant is the deletion, substitution or addition of at least one amino acid residue represented in FIG. 25.

34. A microbial cell according to any of claims 1-33 wherein said microbial cell is a bacterial cell.

35. A microbial cell according to claim 34 wherein said bacterial cell is of the genus Escherichia spp.

36. A microbial cell according to claim 34 wherein said bacterial cell is of the genus Salmonella spp.

37. A vaccine composition comprising a bacterial glycoconjugate antigen polypeptide, or part thereof, as herein described.

38. A composition according to claim 37 wherein said composition includes a carrier and/or optionally an adjuvant.

39. A composition according to claim 37 or 38 comprising a mix of two or three different glycoconjugate antigenic polypeptides as herein described.

40. A cell culture comprising a microbial cell according to any of claims 1-36.

41. A fermentor comprising a microbial cell culture according to claim 40.

42. The use of a cell according to any of claims 1-36 in the production of glycoconjugated polypeptides.

43. A method for the production of a recombinant glycoconjugate polypeptide comprising:
i) providing a microbial culture according to the invention;
ii) culturing the microbial culture; and
iii) isolating the glycoconjugate polypeptide from the micobial cells or the cell culture medium.

44. A method to vaccinate a subject to a bacterial infection comprising immunising said subject with an effective amount of a vaccine according to any of claims 37-39.

45. A vector comprising a nucleotide sequence selected from the group consisting of:
i) a nucleic acid molecule consisting of a nucleic acid sequence as represented in FIG. 1a;
ii) a nucleic acid molecule consisting of a nucleic acid sequence that hybridises under stringent hybridisation conditions to the nucleic acid molecule in (i) and which encodes a polypeptide; wherein said cell expresses a recombinant polypeptide which is a substrate for said oligosaccharyltransferase polypeptide.

46. A vector according to claim 45 wherein said vector includes a nucleotide sequence that encodes an oligosaccharyltransferase polypeptide as represented by the amino acid sequence in FIG. 1b, or a variant polypeptide and comprises the amino acid sequence represented in FIG. 1b which sequence has been modified by deletion, addition or substitution of at least one amino acid residue and which retains or has enhanced oligosaccharyltransferase activity.


SEQUENCE LISTING (99 sequences)

SEQ ID NO: 1 - DNA, 2130 bp, Nitratiruptor tergasus
atgtatgtgcaaaaaaagagcatggtgatgatactggccattgttacagcctatatattttcttatctattccatctttactggattgggtgggcaagtcataaccctgaatttttttggaatggccagttaatgattaataatgtagatggatatttttttggaagtggggcgcaaaaaattttgtatcatgagcatctagataatccaaggctcttggatgtatggcggtacggcacggcagtaattaccgcctatatcgttaaacttctgccaatttctcttgatacggcgatgctttacttacctcctgttcttagttcccttgttgttataccaatcattttggttggtagactctatggcaatgttatatggggttttttgagtgcgcttattgcgtctattggctggagctactacaaccggacattggctggttactacgataccgatatgttcagtgcaatgctacctatgtttgttttcttttttttgatctcagctataaaatctcgctctttaaactatattttagcagcctctttgactgttattctttatccttggctttatgaccaagggctttccattgtttacgcgatgggaattatgatgtttttgtatctggttttgttttatcgctatgaaaagtttacttataaagcgattatcattttttcttttgcactaatgcctattttatggatgtttaaactcttgcttgtgatagttctttggtttctactcaagacttatgatactgagctcaaaaaatttcaaattgctgcaggtgtgagctttttggcatttattttgatagggaatgtttttgggattattttgcataaagttttctcttatacttctcataccgatcaaattgctggtctccattttcttaacgtcaacgaaacggtcagagaggctggtaaaattcctttcgaagtcgtagcggataggattgtaggttctctattggggcttgttattgctgttgcaggatatattttattactcattcgcaataaagagtttattattgcgcttccgttgtgggggataggtttttttgcctattttggaggcctccgatttactgtttatgcagtcccgattgccgcaatgagtgcagtttacttctttttttggctttctgagcgttttgaagccaaaaaggtccgtatagcagcagtgagtctaggtactctttttctcctggctccaaatattacgcatattaccggatgttgcgaaaaggtaagttggcttgatagtatcaaggaattttatcctctaaaaagttatccctatttgacgccaacaactcttttaaaaagtgaagtagctttacttaatgagtttaacaaaaaaagttttcctaaagattacgcaattacgtggtgggattatggatatcctatttggtattatgcagacgttaatactcttatagatggggggaaacacaatgaagataactttttggtttcaaagattcttactacctcagatcaacgcctggctgcaaatctagcgaaactttccatcaaagtatatgtagatacgaacaaaactgttgcaccacaattgtttataaaaaacaaaaaagtgattaatgttgatgaattttttgccaatctttcaacaaaagaatataaaccaccaaaattaaacagagatatttatttaattttacctcacagaatgtttgatatattcccaacagttgcttatttttctgaacgtaatcttaatactgggaaggtttattctaggaaattttattacaaaaatagagtagttcaaaaaggagggaaactttatatcggacaacttatagttgatctaagaagggctgttattcttatcgggaaccagcaggctccaattaaaaacgtctatattataggtattgatcaaaatgggaaaagtcattataaagaacaaaaaattcgtgacaaaggattaagccttattatcaataaaagctttggcgaagctattatagcagataataagtatattaattccatttttttccaaatgtatctatttgataattatgatcctatgctctttgagcctgtgatattaagtccttggatgaaaatttatagaatcaaatga

SEQ ID NO: 2 - protein, 709 aa, Nitratiruptor tergasus
MYVQKKSMVMILAIVTAYIFSYLFHLYWIGWASHNPEFFWNGQLMINNVDGYFFGSGAQKILYHEHLDNPRLLDVWRYGTAVITAYIVKLLPISLDTAMLYLPPVLSSLVVIPIILVGRLYGNVIWGFLSALIASIGWSYYNRTLAGYYDTDMFSAMLPMFVFFFLISAIKSRSLNYILAASLTVILYPWLYDQGLSIVYAMGIMMFLYLVLFYRYEKFTYKAIIIFSFALMPILWMFKLLLVIVLWFLLKTYDTELKKFQIAAGVSFLAFILIGNVFGIILHKVFSYTSHTDQIAGLHFLNVNETVREAGKIPFEVVADRIVGSLLGLVIAVAGYILLLIRNKEFIIALPLWGIGFFAYFGGLRFTVYAVPIAAMSAVYFFFWLSERFEAKKVRIAAVSLGTLFLLAPNITHITGCCEKVSWLDSIKEFYPLKSYPYLTPTTLLKSEVALLNEFNKKSFPKDYAITWWDYGYPIWYYADVNTLIDGGKHNEDNFLVSKILTTSDQRLAANLAKLSIKVYVDTNKTVAPQLFIKNKKVINVDEFFANLSTKEYKPPKLNRDIYLILPHRMFDIFPTVAYFSERNLNTGKVYSRKFYYKNRVVQKGGKLYIGQLIVDLRRAVILIGNQQAPIKNVYIIGIDQNGKSHYKEQKIRDKGLSLIINKSFGEAIIADNKYINSIFFQMYLFDNYDPMLFEPVILSPWMKIYRIK

SEQ ID NO: 3 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 4 - protein, 471 aa, Streptococcus pneumoniae
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAFEWWRTVYEKTDLPLVRKRTISIWGTTLYPQVEDKVEND

SEQ ID NO: 5 - protein, 653 aa, Streptococcus pneumoniae
MNKKKMILTSLASVAILGAGFVASSPTFVRAEEAPVANQSKAEKDYDAAVKKSEAAKKDYETAKKKAEDAQKKYDEDQKKTEAKAEKERKASEKIAEATKEVQQAYLAYLQASNESQRKEADKKIKEATQRKDEAEAAFATIRTTIVVPEPSELAETKKKAEEATKEAEVAKKKSEEAAKEVEVEKNKILEQDAENEKKIDVLQNKVADLEKGIAPYQNEVAELNKEIARLQSDLKDAEENNVEDYIKEGLEQAITNKKAELATTQQNIDKTQKDLEDAELELEKVLATLDPEGKTQDELDKEAAEAELNEKVEALQNQVAELEEELSKLEDNLKDAETNNVEDYIKEGLEEAIATKKAELEKTQKELDAALNELGPDGDEEETPAPAPQPEKPAEEPENPAPAPKPEKSADQQAEEDYARRSEEEYNRLTQQQPPKAEKPAPAPQPEQPAPAPKIGWKQENGMWYFYNTDGSMATGWLQNNGSWYYLNSNGAMATGWLQYNGSWYYLNANGAMATGWLQYNGSWYYLNANGAMATGWLQYNGSWYYLNANGDMATGWLQYNGSWYYLNANGDMATGWAKVHGSWYYLNANGSMATGWVKDGETWYYLEASGSMKANQWFQVSDKWYYVNGLGSLSVNTTVDGYKVNANGEWV

SEQ ID NO: 6 - protein, 487 aa, Streptococcus pneumoniae
MFASKNERKVHYSIRKFSIGVASVAVASLFMGSVVHATEKEVTTQVATSFNKANKSQTEHMKAAKQVDEYITKKLQLDRRKHTQNVGLLTKLGVIKTEYLHRLSVSKEKSEAELPSEIKAKLDAAFEQFKKDTLPTEPGKKVAEAEKKVEEAKKKAEDQKEEDRRNYPTITYKTLELEIAESDVEVKKAELELVKEEAKGSRNEQKVNQAKAKVESKQAEATRLKKIKTDREQAETTRLENIKTDREKAEEAKRKADAKEQDESKRRVKGGVPGEQATLDKKENDAKSSDSSVGEETLPSPSLKSGKKVAEAEKKVAEAEKKAKDQKEEDRRNYPTNTYKTLELEIAESDVKVKEAELELVKEEAKESRNEEKVKQAKAEVESKKAEATRLEKIKTDRKKAEEAKRKAAEEDKVKEKPAEQPQPAPAPQPEKPAPAPKPENPAEQPKAEKPADQQAEEDYARRSEEEYNRLTQQQPPKTEKPAQPST

SEQ ID NO: 7 - protein, 309 aa, Streptococcus pneumoniae
MKKLGTLLVLFLSAIILVACASGKKDTTSGQKLKVVATNSIIADITKNIAGDKIDLHSIVPIGQDPHEYEPLPEDVKKTSEADLIFYNGINLETGGNAWFTKLVENAKKTENKDYFAVSDGVDVIYLEGQNEKGKEDPHAWLNLENGIIFAKNIAKQLSAKDPNNKEFYEKNLKEYTDKLDKLDKESKDKFNKIPAEKKLIVTSEGAFKYFSKAYGVPSAYIWEINTEEEGTPEQIKTLVEKLRQTKVPSLFVESSVDDRPMKTVSQDTNIPIYAQIFTDSIAEQGKEGDSYYSMMKYNLDKIAEGLAK

SEQ ID NO: 8 - protein, 535 aa, Corynebacterium diphtheriae
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 9 - protein, 435 aa, Streptococcus suis
MSIITDVYAREVLDSRGNPTLEVEVYTESGAFGRGMVPSGASTGEHEAVELRDGDKSRYLGLGTQKAVDNVNNVIADAIIGFDVRDQQAIDRAMIALDGTPNKGKLGANAILGVSIAVARAAADYLEVPLYTYLGGFNTKVLPTPMMNIINGGSHSDAPIAFQEFMILPVGAPSFKEGLRWGAEVFHALKKILKARGLVTAVGDEGGFAPKFEGTEDGVETIIEAIEAAGYEAGENGIMIGFDCASSEFYDKERKVYDYTKFEGEGAAVRTSAEQIDYLEELVNKYPIITIEDGMDENDWDGWKALTERLGKRVQLVGDDFFVTNTDYLARGIKEGAANSILIKVNQIGTLTETFEAIEMAKEAGYTAVVSHRSGETEDSTIADIAVATNAGQIKTGSLSRTDRIAKYNQLLRIEDQLGEVAVYKGLNSFYNLKK

SEQ ID NO: 10 - protein, 670 aa, Streptococcus suis
MNTKKWRTSLLIPGIVLFGTVALVNNVSAQEVKNTIISAKQPDGGQATSKAVNVKIPAVVRLFGRELLENEFKFELREANGEELPVLDTAQNTKEGQVRFKNLSFDKPGKYWYTISEVKDELGGIEYDSKYIVAKITVEDRNGQLQAMIEFIDNDNVFNNFYTPAPAAASLSIKKVLEGRTLNTGEFEFVLKNEKGDEIEKVSNQADGSVNFSALTFTKEGTYTYTVSEVDGGLGDIIYDKSDIKATVTVKDNNHGQLVSTVTYENSDQIFENILNPGKLIAPTTDSVITDNEVSKEAMAGKEKGNIEPPKEQIANEEKDNIEASEKQMPSIVNDMVVTPEKQMTNKENDKVVISEKQMPSVVNENAVTPEKQMTNKENDNIETSEKQMPSVVNENAVTPEKQMTNKEKDNIETSEKQMPSVVNENAVTPEKQMTNKEKDNIETSEKQMPSIVNDMVVTPQEQMANKENDKVVISEKQMPSIVNDMVVTPQEQMANKENDKVVISEKQMPSIVNDMVVTPQEQMANKENDKVVISEKQMPSIVNDMVVTPQEQMANKENDKVETSEKQMPVNEKDNAVTPEKQMANKEKENIETSKKQIPVNENNQNGTVEENSNTKPTTEKTDKQETSTFKTETAKQILPVTGEKGSLWLLTSGIIGLAIALFTRKRKL

SEQ ID NO: 11 - protein, 186 aa, Streptococcus suis
MTIKYPDGTIDLLSPVEVVKQADKTAPTVANDGKGNIVIVPSEKAVELVVSYVDNNGKSQTVVVTKGTDGLWTASNTVVIVDPVTGQVIVPGSVIKPGTVVTAYSKDEVGNSSDSAEAEVVAVDENNSAAGVKVKSVTTNANNVEKKAKQLPNTGEEANSATSLGLVALGLGLALLAAKRRRDEEA

SEQ ID NO: 12 - protein, 489 aa, Streptococcus suis
MDFGFDILAIMIPIIVVVLFVLLFVFGYVSAKPNEAIVITGLGKPRTLIGRSGFMIPFIEKRSYISIEQFSTDVQTTDFVPTLDFINVKADAVVKVKVGVSDELLNAAAQNFLNWKTADISASIQDVLEGNLREIIGQMELRDMVNNRQAFAEKVQSNAAPDLAKMGLEIIAFTVQSFTDDNDVIKNLGIDNIVTIQKDAANARAKAEREQAEVRAREDKAANDARVAADLEIAKKQNELAIEQANLKRRSDVQLAQANAAYGIEEQAQRKEIERATAEANIVKQQKEAEVKAEEVKVREQELSATIRKQAEAEKYARQQAAEADLIERQRKAEAELYETQREAEAQKARAEAAKYAAEQEAAGIEAKGRAEAEAIRLKLEAEAEGLSKKADAMAKFNDAAVTEMVVNVLPEIAKNIAAPLGNVDKITMYGEGNASKMVGDLMTTMDKTTEGLGLNVRDLISATLTGRAMSTGLLTAQETKEVEGDKEK

SEQ ID NO: 13 - protein, 266 aa, Streptococcus suis
MFEDPEVDIIYISTPHNTHINYLRKALKAGKHVLCEKSITLNSEELAEAIQLAEENQVVLAEAMTIFHMPIYRQLSEVVASGKLGELKMIQMNFGSYKEYDMTNRFFNKNLAGGALLDIGVYALSFVRWFMIKKPNQVLSQVKLAPTGVDEQVGILLSNDAGKMATIALTLHAKQPKRGTIAYDKGYIELYDYPRGQKAVITYTENGIQKVIEAGETAKALSYEVADMEKAVAGIENTMHLAYTQDVMEIMTQLRKEWGLVYPEKM

SEQ ID NO: 14 - protein, 497 aa, Streptococcus suis
MRKSSHLILSSIVSLALVGVTPLSVLADSKQDINQYFQSLTYEPQEILTNEGEYIDNPPATTGMLENGRFVVLRREKKNITNNSADIAVIDAKAANIYPGALLRADQNLLDNNPTLISIARGDLTLSLNLPGLANGDSHTVVNSPTRSTVRTGVNNLLSKWNNTYAGEYGNTQAELQYDETMAYSMSQLKTKFGTSFEKIAVPLDINFDAVNSGEKQVQIVNFKQIYYTVSVDEPESPSKLFAEGTTVEDLKRNGITDEVPPVYVSSVSYGRSMFIKLETSSRSTQVQAAFKAAIKGVDISGNAEYQDILKNTSFSAYIFGGDAGSAATVVSGNIETLKKIIEEGARYGKLNPGVPISYSTNFVKDNRPAQILSNSEYIETTSTVHNSSALTLDHSGAYVAKYNITWEEVSYNEAGEEVWEPKAWDKNGVNLTSHWSETIQIPGNARNLHVNIQECTGLAWEWWRTVYDKDLPLVGQRKITIWGTTLYPQYADEVIE

SEQ ID NO: 15 - protein, 204 aa, Streptococcus suis
MADVRLYISRHGKTMFNTIGRVQGWCDTPLTKVGEEGIRELGLGLKDAGLDFKLAVSSDLGRTVQTMTIAQRELGILGKIPYYQDKRIREWCFGSFEGMYDAELFQGVLPRLKGTVDATGMSFAEIAAGIQEADTAGWAESWEVLSNRILTGFESIAQDLEKQGGGNALVVSHGMTIATLAHLLEPERGANVFLDNGSITVLKY

SEQ ID NO: 16 - protein, 1150 aa, Streptococcus agalactiae
MRKKQKLPFDKLAIALMSTSILLNAQSDIKANTVTEDTPATEQTVETPQPTAVSEEAPSSKETKTPQTPSDAGETVADDANDLAPQAPAKTADTPATSKATIRDLNDPSQVKTLQEKAGKGAGTVVAVIDAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAINLGAKVINMSFGNAALAYANLPDETKKAFDYAKSKGVSIVTSAGNDSSFGGKTRLPLADHPDYGVVGTPAAADSTLTVASYSPDKQLTETVRVKTADQQDKEMPVLSTNRFEPNKAYDYAYANRGTKEDDFKDVKGKIALIERGDIDFKDKIAKAKKAGAVGVLIYDNQDKGFPIELPNVDQMPAAFISRKDGLLLKDNPQKTITFNATPKVLPTASGTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDKFEVTVNVHNKSDKPQELYYQATVQTDKVDGKHFALAPKVLYEASWQKITIPANSSKQVTVPIDASRFSKDLLAQMKNGYFLEGFVRFKQDPKKEELMSIPYIGFRGDFGNLSALEKPIYDSKDGSSYYHEANSDAKDQLDGDGLQFYALKNNFTALTTESNPWTIIKAVKEGVENIEDIESSEITETILAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETTEGATVPLKMSDFTYVVEDMAGNITYTPVTKLLEGHSNKPEQDGSDQAPDKKPEAKPEQDGSGQTPDKKTETKPEKDSSGQTPGKTPQKGQPSRTLEKRSSKRALATKASTRDQLPTTNDKDTNRLHLLKLVMTTFFLGLVAHIFKTKRQKETKK

SEQ ID NO: 17 - protein, 207 aa, Streptococcus agalactiae
AIKQQTIFDIDNAKTEVEIDNLVHDAFSKMNATVAKFQKGLETNTPETPDTPKIPELPQAPEAPRVPESPKTPEAPHVPESPKAPEAPRVPESPKTPEAPHVPESPKAPEAPRVPESPKTPEAPHVPESPKTPEAPKIPEPPKTPDVPKLPDVPKLPDAPKLPDGLNKVGQAVFTSTDGNTKVTVVFDKPTDADKLHLKEVTTKELA

SEQ ID NO: 18 - protein, 306 aa, Streptococcus agalactiae
MKKVFFLMAMVVSLVMIAGCDKSANPKQPTQGMSVVTSFYPMYAMTKEVSGDLNDVRMIQSGAGIHSFEPSVNDVAAIYDADLFVYHSHTLEAWARDLDPNLKKSKVNVFEASKPLTLDRVKGLEDMEVTQGIDPATLYDPHTWTDPVLAGEEAVNIAKELGHLDPKHKDSYTKKAKAFKKEAEQLTEEYTQKFKKVRSKTFVTQHTAFSYLAKRFGLKQLGISGISPEQEPSPRQLKEIQDFVKEYNVKTIFAEDNVNPKIAHAIAKSTGAKVKTLSPLEAAPSGNKTYLENLRANLEVLYQQLK

SEQ ID NO: 19 - protein, 1055 aa, Streptococcus agalactiae
MTKKHLKTLALALTTVSVVTYSQEVYGLEREESVKQEQTQSASEDDWFEEDNERKTNVSKENSTVDETVSDLFSDGNSNNSSSKTESVVSDPKQVPKAKPEVTQEASNSSNDASKVEVPKQDTASKKETLETSTWEAKDFVTRGDTLVGFSKSGINKLSQTSHLVLPSHAADGTQLTQVASFAFTPDKKTAIAEYTSRLGENGKPSRLDIDQKEIIDEGEIFNAYQLTKLTIPNGYKSIGQDAFVDNKNIAEVNLPESLETISDYAFAHMSLKQVKLPDNLKVIGELAFFDNQIGGKLYLPRHLIKLAERAFKSNRIQTVEFLGSKLKVIGEASFQDNNLRNVMLPDGLEKIESEAFTGNPGDEHYNNQVVLRTRTGQNPHQLATENTYVNPDKSLWRATPDMDYTKWLEEDFTYQKNSVTGFSNKGLQKVRRNKNLEIPKQHNGITITEIGDNAFRNVDFQSKTLRKYDLEEIKLPSTIRKIGAFAFQSNNLKSFEASEDLEEIKEGAFMNNRIGTLDLKDKLIKIGDAAFHINHIYAIVLPESVQEIGRSAFRQNGALHLMFIGNKVKTIGEMAFLSNKLESVNLSEQKQLKTIEVQAFSDNALSEVVLPPNLQTIREEAFKRNHLKEVKGSSTLSQITFNAFDQNDGDKRFGKKVVVRTHNNSHMLADGERFIIDPDKLSSTMVDLEKVLKIIEGLDYSTLRQTTQTQFREMTTAGKALLSKSNLRQGEKQKFLQEAQFFLGRVDLDKAIAKAEKALVTKKATKNGHLLERSINKAVLAYNNSAIKKANVKRLEKELDLLTDLVEGKGPLAQATMVQGVYLLKTPLPLPEYYIGLNVYFDKSGKLIYALDMSDTIGEGQKDAYGNPILNVDEDNEGYHTLAVATLADYEGLYIKDILNSSLDKIKAIRQIPLAKYHRLGIFQAIRNAAAEADRLLPKTPKGYLNEVPNYRKKQMEKNLKPVDYKTPIFNKALPNEKVDGDRAAKGHNINAETNNSVAVTPIRSEQQLHKSQSDVNLPQTSSKNNFIYEILGYVSLCLLFLVTAGKKGKRARK

SEQ ID NO: 20 - protein, 901 aa, Streptococcus agalactiae
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKGILSFILIGGAMMSIAGGIYIWKRYKKSSDMSIKKD

SEQ ID NO: 21 - protein, 554 aa, Streptococcus agalactiae
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 22 - protein, 890 aa, Streptococcus agalactiae
MKKRQKIWRGLSVTLLILSQIPFGILVQGETQDTNQALGKVIVKKTGDNATPLGKATFVLKNDNDKSETSHETVEGSGEATFENIKPGDYTLREETAPIGYKKTDKTWKVKVADNGATIIEGMDADKAEKRKEVLNAQYPKSAIYEDTKENYPLVNVEGSKVGEQYKALNPINGKDGRREIAEGWLSKKITGVNDLDKNKYKIELTVEGKTTVETKELNQPLDVVVLLDNSNSMNNERANNSQRALKAGEAVEKLIDKITSNKDNRVALVTYASTIFDGTEATVSKGVADQNGKALNDSVSWDYHKTTFTATTHNYSYLNLTNDANEVNILKSRIPKEAEHINGDRTLYQFGATFTQKALMKANEILETQSSNARKKLIFHVTDGVPTMSYAINFNPYISTSYQNQFNSFLNKIPDRSGILQEDFIINGDDYQIVKGDGESFKLFSDRKVPVTGGTTQAAYRVPQNQLSVMSNEGYAINSGYIYLYWRDYNWVYPFDPKTKKVSATKQIKTHGEPTTLYFNGNIRPKGYDIFTVGIGVNGDPGATPLEAEKFMQSISSKTENYTNVDDTNKIYDELNKYFKTIVEEKHSIVDGNVTDPMGEMIEFQLKNGQSFTHDDYVLVGNDGSQLKNGVALGGPNSDGGILKDVTVTYDKTSQTIKINHLNLGSGQKVVLTYDVRLKDNYISNKFYNTNNRTTLSPKSEKEPNTIRDFPIPKIRDVREFPVLTISNQKKMGEVEFIKVNKDKHSESLLGAKFQLQIEKDFSGYKQFVPEGSDVTTKNDGKIYFKALQDGNYKLYEISSPDGYIEVKTKPVVTFTIQNGEVTNLKADPNANKNQIGYLEGNGKHLITNTPKRPPGVFPKTGGIGTIVYILVGSTFMILTICSFRRKQL

SEQ ID NO: 23 - protein, 594 aa, Streptococcus agalactiae
MNNNEKKVKYFLRKTAYGLASMSAAFIVCSGIVNTPTVSADSPDTLKVEKLGKLKDVKSVHELTPISIPNELKGAKEQALSSIISHPNITNSEVDKLASDYSFRINTSNDVNDVKRLLNEFYNAVARKQLDTNSADYRSKIDNISTTGLAIALEAKEIYEANKSILPHRYKDSVGTYVNSFEERRSPGKFNIWNGQEGFNAAQKLLEDVKKLLLELQNLTKNNKPNIQVPKQAPTEAAKPALSPEALTRLTTWYNQAKDLLKDDQVKDKYVDILSVQKAVDQAYDHVEEGKFITTDQANQLANKLRDALQSLELKDKKVAKPVAKGTYDVKYVDTEGKEVAKSRHFEGEEGAAFVTSAKEVAGYKLVRTEGAVSNVFTAGAQVRTYVYEKVAKPVAKGTYDVKYVDTEGKEVAKSRHFEGEEGAAFVTSAKEVAGYKLVRTEGAVSNVFTAGAQVRTYVYEKVKPEVKPDVKPEAKPEAKPEVKPDVKPEAKPEAKPEVKSDVKPEAKPEAKPEAKPEVKPDVKPEAKPEAKPATKKSVNTSGNLVAKKAIENKKYSKKLPSTGEAASPLLAIVSLIVMLSAGLITIVLKHKKN

SEQ ID NO: 24 - protein, 249 aa, Streptococcus agalactiae
AIKQQTIFDIDNAKTEVEIDNLVHDAFSKMNATVAKFQKGLETNTPETPDTPKIPELPQAPDTPQAPDTPHVPESPKAPEAPRVPESPKTPDTPHVPESPKAPEAPRVPESPKTPDTPHVPESPKAPEAPRVPESPKTPDTPHVPESPKAPEAPRVPESPKTPEAPHVPESPKTPEAPKIPEPPKTPDVPKLPDVPKLPDAPKLPDGLNKVGQAVFTSTDGNTKVTVVFDKPTDADKLHLKEVTTKELA

SEQ ID NO: 25 - protein, 1231 aa, Streptococcus agalactiae
MFRRSKNNSYDTLQTKQRFSIKKFKFGAASVLIGISFLGGFTQGQFNISTDTVFAAEVISGSAVTLNTNMTKNVQNGRAYIDLYDVKNGKIDPLQLITLNSPDLKAQYVIRQGGNYFTQPSELTTVGAASINYTVLKTDGSPHTKPDGQVDIINVSLTIYNSSALRDKIDEVKKKAEDPKWDEGSRDKVLISLDDIKTDIDNNPKTQSDIANKITEVTNLEKILVPRIPDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNVGETPKAEDSIGNLPDLPKGTTVAFETPVDTATPGDKPAKVVVTYPDGSKDTVDVTVKVVDPRTDADKNDPAGKDQQVNGKGNKLPATGENATPFFNVVALTIMSSVGLLSVSKKKED

SEQ ID NO: 26 - protein, 707 aa, Streptococcus agalactiae
MFRRSKNNSYDTSQTKQRFSIKKFKFGAASVLIGLSFLSGVTQGNLNIFEESIVAASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKYSPEAKHRTVEQHAELDAKDSIANTDELPSNSTYNWKNGHKPDTSTSGEKDGIVEVHYPDGTVDDVNVKVTVTSKKTDNTAPTLTVTPEQQTVKVDEDITFTVTVEDENEVELGLDDLKAKYENDIIGARVKIKYLTKESNKKVMEVTIMKATLADKGAITFTAKDKAGNQAEPKTVTINVLPLKDSNEPKGKDQTVKVGETPKAEDSIGNLPDLPKGTTVAFETPVDTATPGDKPAKVVVTYPDGSKDTVDVTVKVVDPRTDADKNDPAGKDQQVKVGETPKAEDSIGNLSDLPKGTTVAFETPVDTATPGDKPAKVVVTYPDGSKDTVDVTVKVVDPRTDADKNDPAGKDQQVKVGETPKAEDSIGNLPDLPKGTTVAFETPVDTATPGDKPAKVVVTYPDGSKDTVDVTVKVVDPRTDADKNDPAGKDQQVNGKGNKLPATGENATPFFNVAALTIISSVGLLSVSKKKED

SEQ ID NO: 27 - protein, 786 aa, Streptococcus agalactiae
MFRRSKNNSYDTSQTKQRFSIKKFKFGAASVLIGLSFLGGVTQGNLNIFEESIVAASTIPGSAATLNTSITKNIQNGNAYIDLYDVKNGLIDPQNLIVLNPSSYSANYYIKQGAKYYSNPSEITTTGSATITFNILDETGNPHKKADGQIDIVSVNLTIYDSTALRNRIDEVINNANDPKWSDGSRDEVLTGLEKIKKDIDNNPKTQIDIDNKINEVNEIEKLLVVSLPDKIKYSPEAKHRTVEQHAELDAKDSIANTDELPSNSTYNWKNGHKPDTSTSGEKDGIVEVHYPDGTVDDVNVKVTVTSKKTDNTAPTLTVTPEQQTVKVDEDITFTVTAEDENEVELGLDDLKAKYENDIIGARVKIKYLTKEPNKKVMEVTIMKATLADKGAITFTAKDKAGNQAEPKTVTINVLPPDKIKYSPEAKHRTVEQHAELDAKDSIANTDELPSNSTYNWKNGHKPDTSTPGEKNAVVVVTYPDKSTDEVPVKVTVVDPRTDAEKNDPAGKDQTVKVGEQPDPTKSLEAVPAGSTVAYKEPVDTKTPGEKNAIVVVTYPDKSTDEVPVKVTVVDPRTDAEKNDPAGKDQTVKVGEQPDPTKSLEAVPAGSTVAYKEPVDTKTPGEKNAVVVVTYPDKSTDEVPVKVTVVDPRTDAEKNDPAGGETTVPQGTPISDEEITGLVKIPEGSNGVPKVVGDRPNTDVPGDYKVTVEVTYPDGTKDTVAVTVHVTPKPVPDKDKYDPTGKSQQVNGKGNKLPATGESATPFFNVAALTIISSVGLLSVSKKKED

SEQ ID NO: 28 - protein, 204 aa, Klebsiella pneumoniae
MPGRHNGCQSGSLFRHRSKENSMKIKTLAMIVVSALSLSSTAALADTTTVNGGTVHFKGEVVNAACAVDAGSIDQTVQLGQVRSAKLATAGSTSSAVGFNIQLDDCDTTVATKASVAFAGTAIDSSNTTVLALQNSAAGSATNVGVQILDNTGTPLALDGATFSAATTLNDGPNIIPFQARYYATGAATAGIANADATFKVQYE

SEQ ID NO: 29 - protein, 178 aa, Klebsiella pneumoniae
MFKKTTLALLTASLFISTSALAISNNTITFRGEVSDETCSVAINGNQAKPVVILPTVSAKELAKGGDVAGQVTFDIGLTGCTGNTDRATKISTVFVGNQVTSNGNLGNTGDATNVEVQLLDTANKVINLTNGFKGEGDLQLKPGETEASATYTAQYYATGAASAGTVEATLQYAVSYL

SEQ ID NO: 30 - protein, 204 aa, Klebsiella pneumoniae
MAMKKVLLSAAMATAFFGMTAAHAADTNVGGGQVNFFGKVTDVSCTVSVNGQGSDANVYLSPVTLTEVKAAAADTYLKPKSFTIDVSNCQAADGTKQDDVSKLGVNWTGGNLLAGATSKQQGYLANTEASGAQNIQLVLSTDNATALTNKIIPGDSTQPKAKGDASAVADGARFTYYVGYATSAPTTVTTGVVNSYATYEITYQ

SEQ ID NO: 31 - protein, 350 aa, Klebsiella pneumoniae
MKKTAIAIAVALAGFATVAQAAPKDNTWYAGGKLGWSQFHDTGWYNSSLNNNGPTHESQLGAGAFGGYQVNPYLGFEMGYDWLGRMPYKGDQVNGAFKAQGVQLTAKLGYPITDDLDIYTRLGGMVWRADSSNSIAGDNHDTGVSPVFAGGVEWAVTRDIATRLEYQWVNNIGDAGTVGVRPDNGMLSVGVSYRFGQEDAAPVVAPAPAPAPEVTTKHFTLKSDVLFNFNKATLKPEGQQALDQLYTQLSNMDPKDGSAVVMGFTDRIGSEAYNQQLSEKRAQSVVDYLVAKGIPAGKISARGMGKSNPVTGNTCDNVKARAALIDCLAPDRRVEIEVKGYKEVVTQPAA

SEQ ID NO: 32 - protein, 331 aa, Klebsiella pneumoniae
MSLRKLLTLFIVLMALGTTSSWASCTRLSSPTVMLDMVVGRVVVPPDLPVGSVILTRDWTMSAPGGASYRCTSGTNRFAAKIVSPGATDLGNKIYSTNVPGIGMRFSRGGATVNIVYPDVFSSRVYNTTNYSLEGSRFTLEIIKTAATTGSGTLAAGKYTSYDWESGGNPILETYLSANAITVVSPSCSVLSGKNMNVDVGSIRRTDLKGVGTTAGGKDFNIDLQCSGGLSETGYANISTSFSGTLATSTTATMGALLNEKAGSGMAKGIGIQVLKDGSPLQFNKKYTVGRLNNQETRYITIPLHARFYQYGPTTSTGEVESHMIFNLTYD

SEQ ID NO: 33 - protein, 742 aa, Klebsiella pneumoniae
MNNRIKSLALLVNLGIYGVAFPLSAAETATDDKNSAAEETMVVTAAEQNLQAPGVSTITADEIRKRPPARDVSEIIRTMPGVNLTGNSTSGQRGNNRQIDIRGMGPENTLILIDGKPVTSRNSVRLGWRGERDTRGDTSWVPPEMIERIEVIRGPAAARYGNGAAGGVVNIITKKTGDEWHGSWNTYMNAPEHKDEGSTKRTNFSLSGPLGGDFSFRLFGNLDKTQADAWDINQGHQSERTGIYADTLPAGREGVQNKNIDGLVRWEFAPMQSLEFEAGYSRQGNLYAGDTQNTNSNDLVKENYGKETNRLYRNTYSVTWNGAWDNGVTTSNWAQYERTRNSRKGEGLAGGTEGIFNSNQFTDIDLADVMLHSEVSIPFDYLVNQNLTLGSEWNQQRMKDNASNTQALSGGTIPGYDSTGRSPYSQAEIFSLFAENNMELTDTTMLTPALRFDHHSIVGNNWSPSLNLSQGLWDDFTLKMGIARAYKAPSLYQTNPNYILYSKGQGCYASKDGCYLQGNEDLKAETSINKEIGLEFKRDGWLAGVTWFRNDYRNKIEAGYAPVYQNNKGTDLYQWENVPKAVVEGLEGTLNVPVSETVNWTNNITYMLQSKNKETGDRLSIIPEYTLNSTLSWQVRDDVSLQSTFTWYGKQEPKKYNYKGQPVTGSEKNEVSPYSILGLSATWDVTKYVSLTGGVDNVFDKRHWRAGNAQTTGGATGTMYGAGAETYNESGRTWYLSVNTHF

SEQ ID NO: 34 - protein, 363 aa, Klebsiella pneumoniae
MKVKVLSLLVPALLVAGAANAAEIYNKDGNKLDLYGKIDGLHYFSDDKDVDGDQTYMRLGVKGETQINDQLTGYGQWEYNVQANNTESSSDQAWTRLAFAGLKFGDAGSFDYGRNYGVVYDVTSWTDVLPEFGGDTYGSDNFLQSRANGVATYRNSDFFGLVDGLNFALQYQGKNGSVSGEGATNNGRGALKQNGDGFGTSVTYDIFDGISAGFAYANSKRTDDQNQLLLGEGDHAETYTGGLKYDANNIYLATQYTQTYNATRAGSLGFANKAQNFEVAAQYQFDFGLRPSVAYLQSKGKDLNGYGDQDILKYVDVGATYYFNKNMSTYVDYKINLLDDNSFTRSAGISTDDVVALGLVYQF

SEQ ID NO: 35 - protein, 170 aa, Klebsiella pneumoniae
MNKIARLSALAVVLAASVGTTAFAATSTVTGGYAQSDMQGKANKAGGFNLKYRYEQDNNPLGVIGSFTYTEKDNNSNGTYNKGQYYGITAGPAYRLNDWASIYGVVGVGYGKFQNNNYPHKSDMSDYGFSYGAGLQFNPIENVALDFSYEQSRIRNVDVGTWIAGVGYRF

SEQ ID NO: 36 - protein, 212 aa, Klebsiella pneumoniae
MKKLAAAALILSTLSTGSVWAHEAGEFFIRAGTATVRPTEGSDNVLGSLGSFNVSNNTQLGLTFTYMATDNIGVELLAATPFRHKVGTGPTGTIATVHQLPPTLMAQWYFGDAQSKVRPYVGAGINYTTFFNEDFNDTGKAAGLSDLSLKDSWGAAGQVGLDYLINRDWLLNMSVWYMDIDTDVKFKAGGVDQKVSTRLDPWVFMFSAGYRF

SEQ ID NO: 37 - protein, 521 aa, Streptococcus iniae
MAKQIKARKHALRKMITSAVLAGTAITTIGGAMGSVTTVKADSDRLTLEEKMEALRKVVTREVLIGYANNNPRFGFWMSLQQLEKEIDKTQRLTWENKRMEETLKSKVERINEAGVLLSKKQKDLNEAEAKITDLNSKQTDLTNQKEQVEKELKDTKDKLKDSIANASRIAEHEAIRSAGLENQVKSLRDVTKSLVSTINTLTKESATIKAKMAEIQEEANKKIAAVQTELENIDSENQSLSTANEQLKADLEQAARELDTLQSSYYTVENEKAELQKQLAEKDAKIAELEANNTELTATVADLTKALEAAKKEAEEKPALKAKVAELEKALAEAKGLGTKVAELEKDLEKAQAEAKDLETKLAETKAELEKVQAEKAELEATIEKMKKEHAEELDKLNALLADKEKLVEALNKEIEALKKDFDEKSNHSAQEKAKFQEELERLKKELAAKINMPMGNTKGMANAAGNAQTPANNGQNNAVKNQLPSTGDKAGNPFFTASAIAVMVGAGTLAYGRKRKEEE

SEQ ID NO: 38 - protein, 1122 aa, Streptococcus iniae
MKSKVHSKKLIMLQLSLAATSVLLTHVTSVSAAETTAETAISTHVETVGLDLIDDLADVSETVVSAQPSSGEASVEPVSGEEQKPLIDYVDPSNVKDIWEKVGRGKGSLIAVIDAGIEQTHDMLNLADSSDLKYSSKEDLEAKKKEHGIERGKWVNHKLVFYHDYNEGVDSPKSGDEDLYHGTHVAGIAAGSMVNNKKNELLMEGIAPDAQLMFMRVGKTSLIPEKENLYALAIEDAVALGATAINMSFGSVGKASDELKESVHRALNAAREKGVALIVAAGNDFAMGGYPVKPLAKNPDFGVIGTPATTDDVLTIAAYVAPEDISEVFTVASHNDSKELAVTVASAFPKGRQLDFIDIGKGLEDDYLDKDVKGKIVIVDYEAVITSKATAELAQSKGVAGVLVHHRDYKRPLLPLNYHGDLPMGFISLEDFDYLKSLDKATLTFNHKKKLVSVPGGRQMANFSSWGLSADGHMKPDLSAPGYELYSPSLKNTYEPMSGTSSASPHAMGIVSLVQEHVKKKYPQYSPQEQLLLVKNILMSTADPIISPVDNTYYSPRLQGAGAIDAKKAIATDVYLTGSNGLSKINLGDISNTFDLKVRLHNMGNQTKQFKYYATVLADKAENGKMTLRPQKLYTTEQEEVILAPNEEKEVSLTIDISNFDASLIAQMINGYFVDGFVHFDSNDGIKNVLSIPFLGFKGTFADLEALDSPIYNSLDGTFYYTPEEGQDKEDFEVDSIQQIKNNHFTGLVTTFTPWSLVEGKKIEGFDIESASEIPLDDFLGSYVKDGDPTIRRFHFKDGKAYLAISPNGDNNMDSLAFKGIFLRNVKDIKAQVFASDHLDSPIWESQATPFAQKHVNTDELKEGVLENTKWDGKDSSGNIVKEGEYVYRITYTPIAKGAKEQSVEFTVLVDLTLPEIPENILFDSNERTLDIPKLSNSKSNDVYRDRLYYRYGDESVSFTFFDRDENGQFKLPEEIEDELSGEMISIDITKLDQFFYVLEDRAGNYNVLSLEALLKMNQMSDGILEDHQEQALPESDNPLELDKKTPLLSAVSQEVAKEAKVAIKTLQAARPSATLTKASQEKDSNLPVTNDSQKSHKLLGSVFLISALVLGLVSYFKRKSH

SEQ ID NO: 39 - protein, 536 aa, Human papillomavirus
MSLWLPSEATVYLPPVPVSKVVSTDEYVARTNIYYHAGTSRLLAVGHPYFPIKKPNNNKILVPKVSGLQYRVFRIHLPDPNKFGFPDTSFYNPDTQRLVWACVGVEVGRGQPLGVGISGHPLLNKLDDTENASAYAANAGVDNRECISMDYKQTQLCLIGCKPPIGEHWGKGSPCTNVAVNPGDCPPLELINTVIQDGDMVDTGFGAMDFTTLQANKSEVPLDICTSICKYPDYIKMVSEPYGDSLFFYLRREQMFVRHLFNRAGTVGENVPDDLYIKGSGSTANLASSNYFPTPSGSMVTSDAQIFNKPYWLQRAQGHNNGICWGNQLFVTVVDTTRSTNMSLCAAISTSETTYKNTNFKEYLRHGEEYDLQFIFQLCKITLTADVMTYIHSMNSTILEDWNFGLQPPPGGTLEDTYRFVTSQAIACQKHTPPAPKEDPLKKYTFWEVNLKEKFSADLDQFPLGRKFLLQAGLKAKPKFTLGKRKATPTTSSTSTTAKRKKRKLYRLRGPGRAFVTITFKQIPNPLLGLDRCRRS

SEQ ID NO: 40 - protein, 428 aa, Human papillomavirus
AVVNTDDYVTRTSIFYHAGSSRLLTVGDPYFRVPAGGGNKQDIPKVSAYQYRVFRVQLPDPNKFGLPDTSIYNPETQRLVWACAGVEIGRGQPLGVGLSGHPFYNKLDDTESSHAATSNVSEDVRDNVSVDYKQTQLCILGCAPAIGEHWAKGTASKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGVCWHNQLFVTVVDTTRSTNLTICASTQSPVPGQYDATKFKQYSRHVEEYDLQFIFQLCTITLTADVMSYIQSMNSSILEDWNGGSGGNKDPYDKLKFWNVDLKEKFSLDLDQYPLGRKFLVQAGL

SEQ ID NO: 41 - protein, 168 aa, Pseudomonas aeruginosa
MEMLKFGKFAALALAMAVAVGCSSKGGDASGEGANGGVDPNAGYGANSGAVDGSLSDEAALRAITTFYFEYDSSDLKPEAMRALDVHAKDLKGSGQRVVLEGHTDERGTREYNMALGERRAKAVQRYLVLQGVSPAQLELVSYGKERPVATGHDEQSWAQNRRVELKK

SEQ ID NO: 42 - protein, 350 aa, Pseudomonas aeruginosa
MKLKNTLGVVIGSLVAASAMNAFAQGQNSVEIEAFGKRYFTDSVRNMKNADLYGGSIGYFLTDDVELALSYGEYHDVRGTYETGNKKVHGNLTSLDAIYHFGTPGVGLRPYVSAGLAHQNITNINSDSQGRQQMTMANIGAGLKYYFTENFFAKASLDGQYGLEKRDNGHQGEWMAGLGVGFNFGGSKAAPAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVEAEVEAEAK

SEQ ID NO: 43 - protein, 488 aa, Pseudomonas aeruginosa
MALTVNTNIASLNTQRNLNASSNDLNTSLQRLTTGYRINSAKDDAAGLQISNRLSNQISGLNVATRNANDGISLAQTAEGALQQSTNILQRIRDLALQSANGSNSDADRAALQKEVAAQQAELTRISDTTTFGGRKLLDGSFGTTSFQVGSNAYETIDISLQNASASAIGSYQVGSNGAGTVASVAGTATASGIASGTVNLVGGGQVKNIAIAAGDSAKAIAEKMDGAIPNLSARARTVFTADVSGVTGGSLNFDVTVGSNTVSLAGVTSTQDLADQLNSNSSKLGITASINDKGVLTITSATGENVKFGAQTGTATAGQVAVKVQGSDGKFEAAAKNVVAAGTAATTTIVTGYVQLNSPTAYSVSGTGTQASQVFGNASAAQKSSVASVDISTADGAQNAIAVVDNALAAIDAQRADLGAVQNRFKNTIDNLTNISENATNARSRIKDTDFAAETAALSKNQVLQQAGTAILAQANQLPQAVLSLLR

SEQ ID NO: 44 - protein, 488 aa, Pseudomonas aeruginosa; identical to SEQ ID NO: 43

SEQ ID NO: 45 - protein, 709 aa, Pseudomonas aeruginosa
MSEQNNEQRSQAAGTDTVDRGNSNAKLEQLEAYREDATGEALSTNTGTRIADNQNTLKAGERGPSLLEDFIMREKITHFDHERIPERVVHARGSAAHGYFEAYEDLSDLTKAGFLAEAGKRTPVFVRFSTVQGPRGSADTVRDVRGFAVKFYTDEGNFDLVGNNMPVFFIQDAIKFPDFVHAVKPEPHNEIPTGASAHDTFWDFVSLTPESAHMVMWLMSDRAIPIAYRNMQGFGVHTFRLVNAAGESVLVKFHWRPKSGTCSLVWDEAQKLAGKDPDFNRRTLWEDIEKGDYPEWELGLQVIPENQQDSFDFDLLDPTKLVPEELVPVRVVGRMVLNRNPDNFFAETEQVAFHVGHVVPGIDFTNDPLLQGRLFSYTDTQLLRLSGPNFNEIPINRPLCPFHNNQRDAPHRQTINRGRASYEPNSIDGGWPKETPPAARNGGFSTYHEPVSGSKLRKRADSFADHFSQAALFWHSMSEAEQAHIVAAYSFELSKVERQSIREREVNQILLNIDPQLAARVAANVGVQLAAPANPTPQPKPSPALSQMNLLSGDIRSRKVAILIADGVAESDVSDLRDALRQEGADAKLIAPSASPVQAENGAELSPEGTWDGLPSVAFDAVFVPGGAASSQAIGADGRGLHYLLEAYKHLKPVAFAGDAQALASQLSLPGDPGVVLGATATDVFPGLRQALMQHRIWQREAATKAIPA

SEQ ID NO: 46 - protein, 482 aa, Pseudomonas aeruginosa
MEEKTRLTTAAGAPVVDNQNVQTAGPRGPMLLQDVWFLEKLAHFDREVIPERRMHAKGSAAYGTFTVTHDITPYTRAKIFSQVGKKTDMFLRFSTVAGERGAADAERDIRGFSMRFYTEQGNWDLVGNNTPVFYLRDPLKFPDLNHVVKRDPRTNLRNATFKWDFFSHLPESLHQLTIDFSDRGLPKSYRHIHGFGSHTFSFINANNERFWVKFHFKTQQGIENLTNAEAAEVIAQDRESSQRDLYESIEKGDFPRWKMYVQIMPEKEAATYRYNPFDLTKVWPHGDYPLIEVGFFELNRNPDNYFAEVEQAAFTPANVVPGIGFSPDKMLQGRLFSYGDAHRYRLGVNHHQIPVNAARCPHQVYHRDGGMRVDGNNAHQRVTYEPNSFNQWQEQPDFSEPPLSLEGAADHWNHRVDDDYYSQPAALFHLFTDEQKQRLFANIAEDIRDVPEQIQRRQIGLFLKVDPAYGKGVADALGLKLD

SEQ ID NO: 47 - protein, 485 aa, Pseudomonas aeruginosa
MKPNPPINELVLLQAHQLAERIRLRQVSCREVMQAYLAHIERFNPRVNALVSLLPAERLLEEADARDGELARGEWRGWMHGLPHAIKDLSLTRGIRTTLGSPLYRDYIPERDGIMVERIRAAGAILIGKTNTPEFGLGSQTYNPLFGATACAYDERLSAGGSSGGAAAALALHLVPVADGSDMMGSLRNPAAFNNVIGFRPSQGRVPFDDSADLFFDQLGYEGPMGRSVRDTALLLSVQAGADRRAPLSIAEPGARFAAPLERDFEGTRLGWLGDLDGYLPMQDGILQMCQRALGDFRSLGCEIEEANLGFAPERLWECWRTLRHWLVAGSLGGVHADPAKRALLKPEALWEVENGLRLGADAVFRASVIRSDWYRAIGRLFERYDYLLLPSAQVFPFAKETPWPRSIEGVAMDTYHRWMEVVVPGSLSGCPVANVQAGFNAEGLPMGLQIIGPHQADFAVLQLAHAYERASRWFERQPSPLLAN

SEQ ID NO: 48 - protein, 797 aa, Pseudomonas aeruginosa
MKRFLLPALLSALMIAEVHAESFTVSDIRVNGLQRVSAGSVFAALPLNVGETIDDQALVQATRSLFKTGFFQDIQLGRDGNVLVVTVVERPSISSIEIEGNKAISKEDLLKGLKQSGLAEGEIFQRATLEGVRNELQRQYVAQGRYSAEINAEVIPQPRNRVALKININEGTVAAISHINVVGNTVFSEEDLTDLFELKTTNWLSFFKNDDKYAREKLSGDLERLRSYYLDRGYINMDIASTQVSITPDKKHVYITVNINEGEKYTIRDVKLTGDLKVPEEEVKRLLLVQKGQVFSRKVMTTTSDLITRRLGNEGYTFANVNGVPEAHDDDKTVSVTFVVDPGKRAYVNRINFRGNTKTEDEVLRREMRQMEGGWASTYLIDQSKARLERLGYFKEVNVETPAVPGTDDQVDVNYSVEEQPSGSITASVGFAQSAGLILGGSISQNNFLGTGNKVSIGLTRSEYQTRYNFGFVDPYWTVDGVSLGYNAFYRKTDYDELDVDVASYSVNSLGAGMSIGYPISETSRLTYGLSVQRDQIDTGRYTVDEIYDFLDKEGDNFTNFKASIGWSESTLNKGVLATRGHSQSLTLETTLPGSDLSFYKIDYRGQVFAPLTDNYTMRFHTELGYGDGYGSTERLPFYENYYAGGFNSVRGFKDSTLGPRSTPSVARNPDGTPMKNQGPDSKGRYTDPDQDPEAFGGNILITGGAELLFPLPFVKDQRQLRTVLFWDVGSTFDTDCPTKTTTNCDGIKTDNLASSVGVGLTWITALGPLSFSLATPIKKPDNAETQVFQFSLGQTF

SEQ ID NO: 49 - protein, 294 aa, Pseudomonas aeruginosa
MEVRNLNAARELFLDELLAASAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSAKQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLRDILSAI

SEQ ID NO: 50 - protein, 226 aa, Pseudomonas aeruginosa
ALLERNYPTGAEFLGDSGDVSFNTRGTQNWTVERLLQAHRQLEERGYVFVGYHGTFLEAAQSIVFGGVRARSQDLDAIWRGFYIAGDPELAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 51 - protein, 1417 aa, Pseudomonas aeruginosa
MNKSYTLVWNQATGCWNVASEGTRRRSKSGRGKALVVAGASLLGLFCQAPAFALPSGATVVSGDAGFQTSTDGRHMVIDQQSHKLITNWNEFSVRADERVSFHQPGQDAVALNRVIGRNGSDIQGRIDANGKVFLVNPNGVVFGKSAQVNVGGLVASTLDLADRDFLAGNYQFSGDSGATVSNAGSLKASEGGSIALLGARVSNDGVIQAQLGAVALGAGQGINLNFDGDGLLNLQVDKGSVDALAHNGGLIRADGGQVLMSARSADSLLKTVVNNQGTLEARTLRSAEGRIVLDGGEQGTVRVAGKQDASAIGGGNGGLVLNQGANVEIQRTAQVDTHADQGATGTWRILSHEVSVAAVGQANAAGDGSGQVHVAQGPAGANASDSNGVTIVQQQPAVDLAAGANGTSAVQSQSGANIGSGASGISVVQSQNSPNIGSGANGISVVQSQNGANIGAGASGISVVQSQNSPNIGSGVNGVTVVQSQNGANIGSGASGITVVQSQNGANIGSGASGISVVQSQSGPSIGSGVNGVTIVQSQSGANIGPGVSGIDVVQTQTLPNLSPGANGSSIVQVQTLPDIAADAGNVHVVQVQTGGNKVFGNSATNVRSRTVQARSSENVGSGLANPSSAGKGPTLHADTLARNLSTSNVEVVATRGNAHVGAPLSWDSGNGLTLTAERGDLRINGALTAQGENASLTLNAGQRPLRIDDSLSLTGQGARVEFNSDKGYALAEGARITLSGKNAGFRANGRDYSVIQDLQQLRGIDRDLGGSYVLGNRIAGGNSSFLSIGNASAFGGTFDGLGNTIDNLAVYGTGAYSGLFSVNRGTLRNLNLERISADGAQATHYNVQVGSLAAVNLGRIDNVNASDIRIAAASKLNSLGGLVALNLGSIDNASASGTLVGNRHTYALGGLAAENISTARGVASISNSRADFAISGQLKDHASHYGAGGLVGRNRGGLIRSSGSQGTLSLSGHGMNLGGLVGYSSAGGLADVSAFVDVSGNGQHGLYGGLIGLNVNSGIAHATASGKVRGTDAEALGGLIGRNLNAAITNASAHGDVVLQAGRYLGGLIGHNQAGNLADVSASGNLSGGSLLQAGGLIGLNANASLVNASAKGNVATRGAEAVGGLLGENLYGSIINGSASGEVTDGSGKTLGGLIGSNLGGNHSNLKASGWVNAGANSDVGGLIGHNRGGNHSTLAASGNVTGGKGSRVGGLVGYNDAASLTNVSASGNVSANGSRAIGGLLGSDLRGSLMLASSYGTVIDMTGHNLGGLLGRGENTSIRSANATGAVTGGGGASVGGLVGSLEGWRALVLGASASGDARAGYDSYIGGLAGFSTGTIRGASASGKVGGSGLLGGLVAWNQGNVMGSSASGRLEPQIPNQIHGGLIGINFGWQSWNSVYGAAATVPMIGRHYNL

SEQ ID NO: 52 - protein, 721 aa, Actinebacter baumanni
MRIIYIIFNLMNIPPRSFKLTVIACAICYANLTYAQDAQVQALQTIQVKASNAEQSSEQTKAYNVKNSSSATKLNIEAKETPQTINVVTRQQIEDFGLTSTRDVLKNTPGVTVSNQETERTTYMARGFEISNILTDGVGFPLSGYNYNNTNPDTYFYDRVEVVKGADSLTNAFGDPSATINNIRKRPTQEFQASGGVSYGSWDTQRYEADVSGSILPSGKVRGRIMGYEQTGDSYLDRYSAEKNGFAGIVEADLTDSTLLTAVNSQEQNKPNANNWGALPLLDANGKQISYDRSYNPNPDWAHWDNETQNAFVELKQKLNDQWNAKLTYNYLDTKHNSRLLYYYGYPKSDGSGVSLTPWGGQEHQEKHAVDFNLEGTYKLFNREHEATLGYSYVRNHQQDKQSTGTINDSNVIKSTTTDWASWTPQSITWSDFTEAANYKQNINSIYAATRLHLNEDLKLLLGANYVQAESKGESYGSPMSYSESKVSPYVGLTYNFTPEYTGYMSYTSIFRPQTGIDKDTNQALKPIEGKSYEMGVKSSWLDDRLTGTLSVFKIETKHYLLRNSDGNPLNRKVPTSDLESQGVEVGLSGQITDNVNLSLGYAQFSIKDTKNGGDEEPYNPNQTLNLLTTYTPPVLPKLKVGAGLQWQDGIKLYDSNVNGTIKQDAYALVNLMASYEVNDHITLQANGNNIFDKKYLNSFPDGQAFYGAPANYTVAVKFKY

SEQ ID NO: 53 - protein, 362 aa, Salmonella enterica
MKLKLVAVAVTSLLAAGVVNAAEVYNKDGNKLDLYGKVHAQHYFSDDNGSDGDKTYARLGFKGETQINDQLTGFGQWEYEFKGNRTESQGADKDKTRLAFAGLKFADYGSFDYGRNYGVAYDIGAWTDVLPEFGGDTWTQTDVFMTGRTTGVATYRNTDFFGLVEGLNFAAQYQGKNDRDGAYESNGDGFGLSATYEYEGFGVGAAYAKSDRTNNQVKAASNLNAAGKNAEVWAAGLKYDANNIYLATTYSETLNMTTFGEDAAGDAFIANKTQNFEAVAQYQFDFGLRPSIAYLKSKGKNLGTYGDQDLVEYIDVGATYYFNKNMSTFVDYKINLLDDSDFTKAAKVSTDNIVAVGLNYQF

SEQ ID NO: 54 - protein, 561 aa, Salmonella enterica
MQIQSFYHSASLKTQEAFKSLQKTLYNGMQILSGQGKAPAKAPDARPEIIVLREPGATWGNYLQHQKASNHSLHNLYNLQRDLLTVAATVLGKQDPVLTSMANQMELAKVKADRPATKQEEAAAKALKKNLIELIAARTQQQDGLPAKEAHRFAAVAFRDAQVKQLNNQPWQTIKNTLTHNGHHYTNTQLPAAEMKIGAKDIFPSAYEGKGVCSWDTKNIHHANNLWMSTVSVHEDGKDKTLFCGIRHGVLSPYHEKDPLLRHVGAENKAKEVLTAALFSKPELLNKALAGEAVSLKLVSVGLLTASNIFGKEGTMVEDQMRAWQSLTQPGKMIHLKIRNKDGDLQTVKIKPDVAAFNVGVNELALKLGFGLKASDSYNAEALHQLLGNDLRPEARPGGWVGEWLAQYPDNYEVVNTLARQIKDIWKNNQHHKDGGEPYKLAQRLAMLAHEIDAVPAWNCKSGKDRTGMMDSEIKREIISLHQTHMLSAPGSLPDSGGQKIFQKVLLNSGNLEIQKQNTGGAGNKVMKNLSPEVLNLSYQKRVGDENIWQSVKGISSLITS

SEQ ID NO: 55 - protein, 548 aa, Salmonella enterica
MAAKDVKFGNDARVKMLRGVNVLADAVKVTLGPKGRNVVLDKSFGAPTITKDGVSVAREIELEDKFENMGAQMVKEVASKANDAAGDGTTTATVLAQSIITEGLKAVAAGMNPMDLKRGIDKAVAAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKIADLKGQNEDQNVGIKVALRAMEAPLRQIVLNCGEEPSVVANTVKGGDGNYGYNAATEEYGNMIDMGILDPTKVTRSALQYAASVAGLMITTECMVTDLPKSDAPDLGAAGGMGGMGGMGGMM

SEQ ID NO: 56 - protein, 185 aa, Salmonella enterica
MKNIILSTLVITTSVLVVNVAQADTNAFSVGYAQSKVQDFKNIRGVNVKYRYEDDSPVSFISSLSYLYGDSQDSGSIESEGIHYHDKFEVKYGSLMVGPAYRLSDNFSLYALAGVGTVKATFKEHATQDGDSFSNKISSRKTGFAWGAGVQMNPLENIVVDVGYEGSNISSTKINGFNVGIGYRF

SEQ ID NO: 57 - protein, 191 aa, Salmonella enterica
MYTECTYITVINNKARLFFMNMKTSFIAAAVALATVYSFSVSAVQKDITVTANIDSTLELLQADGSSLPSTMKLDFMPGKGLVHKSLQTRLYSNDQTKSVNVKLLNAPQLINVLDPTKTIDMEVTLGGRSLTTTNSVLEAKTLFPDGKTGDASALLNLDIGQKAGAALQNLPAGEYSGLVSLVISQAVTAG

SEQ ID NO: 58 - protein, 379 aa, Salmonella enterica
MAKRDYYEILGVSKTAEEREIKKAYKRLAMKYHPDRNQGDKEAEAKFKEIKEAYEVLTDAQKRAAYDQYGHAAFEQGGMGGGFGGGFNGGADFSDIFGDVFGDIFGGGRGRQRAARGADLRYNMDLTLEEAVRGVTKEIRIPTLEECDVCHGSGAKAGTQPQTCPTCHGSGQVQMRQGFFAVQQTCPHCQGRGTLIKDPCHKCHGHGRVEKSKTLSVKIPAGVDTGDRIRLAGEGEAGEHGAPAGDLYVQVQVKQHPIFEREGNNLYCEVPINFAMAALGGEIEVPTLDGRVMLKVPSETQTGKLFRMRGKGVKSVRGGAQGDLLCRVVVETPVGLSEKQKQLLKDLQESFGGPTGEKNSPRSKSFFDGVKKFFDDLTR

SEQ ID NO: 59 - protein, 376 aa, Salmonella enterica
VKVLSLLVPALLVAGAANAAEIYNKDGNKLDLFGKVDGLHYFSDDKGSDGDQTYMRIGFKGETQVNDQLTGYGQWEYQIQGNQTEGSNDSWTRVAFAGLKFADAGSFDYGRNYGVTYDVTSWTDVLPEFGGDTYGADNFMQQRGNGYATYRNTDFFGLVDGLDFALQYQGKNGSVSGENTNGRSLLNQNGDGYGGSLTYAIGEGYSVGGAITTSKRTADQNNTANARLYGNGDRATVYTGGLKYDANNIYLAAQYSQTYNATRFGTSNGSNPSTSYGFANKAQNFEVVAQYQFDFGLRPSVAYLQSKGKDISNGYGASYGDQDIVKYVDVGATYYFNKNMSTYVDYKINLLDKNDFTRDAGINTDDIVALGLVYQF

SEQ ID NO: 60 - protein, 363 aa, Salmonella enterica
MMKRKILAAVIPALLAAATANAAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMSIVENTVTDTVEMANKTQNLEVVAQYQFDFGLRPAISYVQSKGKQLNGAGGSADLAKYIQAGATYYFNKNMNVWVDYRFNLLDENDYSSSYVGTDDQAAVGITYQF

SEQ ID NO: 61 - protein, 377 aa, Salmonella enterica
MTIKITALAASIGAAVAFMPFATQAEITVLKQDPQAGNPLSRLNFTVGGSIRPQFQNMTGNDGANGYKRNGFDGGTRFRFAADYYLFDDISWISYYELGVNIPAVFDWDNHYAKGTTDTTRRMLYTGFKSATWGTLTFGQQNSIYYDVVGVKTDIWDYDMIGQAPGNGINGDYDGSYRTRKSLKYKKSIGNVDLYASYLFSDDYNANNGLNYKRKGGGSLGADYHLTDDLTWGTAWNYTRAEMRGNGNKTYDQNIVGTALSWTPENWTIAAGGGWYQNFMTTKKVSVNDYFAGDAWGLEYFVGYAFPIGQYAVKAIQPYFMGDRIEYVNGRDYLRTDNGVGITFQLDYGFRVDYEHVFTSSTDNLGDMNLVRLRYDF

SEQ ID NO: 62 - protein, 764 aa, Bacillus anthracis
MKKRKVLIPLMALSTILVSSTGNLEVIQAEVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 63 - protein, 259 aa, Campylobacter jejuni
MVFRKSLLKLAVFALGACVAFSNANAAEGKLESIKSKGQLIVGVKNDVPHYALLDQATGEIKGFEVDVAKLLAKSILGDDKKIKLVAVNAKTRGPLLDNGSVDAVIATFTITPERKRIYNFSEPYYQDAIGLLVLKEKKYKSLADMKGANIGVAQAATTKKAIGEAAKKIGIDVKFSEFPDYPSIKAALDVKRVDAFSVDKSILLGYVDDKSEILPDSFEPQSYGIVTKKDDPAFAKYVDDFVKEHKNEIDALAKKWGL

SEQ ID NO: 64 - protein, 245 aa, Campylobacter jejuni
MKKILILSLAASFLNAEILVYGPGGPAPVLKELALKFEEKTKEKVIVTAGPTPAWVDKAKENADLIFSGNTSMMDDFAKKIPSLSLENLSVLNVRPSGIIVRPNNPKNIKNFEDILKDGINVMVVDGAGQVGLYEDMALKSAKRENLVKLRKNIKIYAKNSKAAVDEWNNNPNIDALIIWSHWAKALGDDKALFIKDKNAVIYRAAEIAPTKKGLENKKALEFVDFIKSQEAQKVWKKYTWKEVK

SEQ ID NO: 65 - protein, 250 aa, Campylobacter jejuni
MKKIITLFGACALAFSMANADVNLYGPGGPHTALKDIANKYSEKTGVKVNVNFGPQATWFEKAKKDADILFGASDQSALAIASDFGKDFNVSKIKPLYFREAIILTQKGNPLKIKGLKDLANKKVRIVVPEGAGKSNTSGTGVWEDMIGRTQDIKTIQNFRNNIVAFVPNSGSARKLFAQDQADAWITWIDWSKSNPDIGTAVAIEKDLVVYRTFNVIAKEGASKETQDFIAYLSSKEAKEIFKKYGWRE

SEQ ID NO: 66 - protein, 315 aa, Campylobacter jejuni
MKKIFTVALLGATLLYAESSAFGAGDITSNSSYGLTSNEKLFKEKLDNLNNENIQTNARINEINERIEGLQSTLEGINSQYAKSNSRLSQVEENNQNIENNFTSEIQKLKAYVEESRKIQEANNKQVKKVLAELSSLVDAINANYVSKNELNDANLSVKTITPSVVVSTTDSNSTIENNNTQNTQDDKAKQIDESWKKKKNNEILELAIKDVDKNAFEDSKAKLNFLITKQYKPARANFWLGEIEYKQKNYNNAIVYYKKSSSLSTKGDYFPKLLYHTAISLDKTGDTKTANGFYKALKTNYPNSPEAKASPNRK

SEQ ID NO: 67 - protein, 279 aa, Campylobacter jejuni
MKKMLLSIFTTFVAVFLAACGGNSDSGASNSLERIKQDGVVRIGVFGDKPPFGYVDEKGVNQGYDIVLAKRIAKELLGDENKVQFVLVEAANRVEFLKSNKVDIILANFTQTPERAEQVDFCLPYMKVALGVAVPQDSNISSIEDLKDKTLLLNKGTTADAYFTKEYPDIKTLKYDQNTETFAALIDQRGDALSHDNTLLFAWVKEHPEFKMAIKELGNKDVIAPAVKKGDKELKEFIDNLITKLGEEQFFHKAYDETLKSHFGDDVKADDVVIEGGKI

SEQ ID NO: 68 - protein, 572 aa, Campylobacter jejuni
MGFRINTNVAALNAKANADLNSKSLDASLSRLSSGLRINSAADDASGMAIADSLRSQANTLGQAISNGNDALGILQTADKAMDEQLKILDTIKTKATQAAQDGQSLKTRTMLQADINRLMEELDNIANTTSFNGKQLLSGNFINQEFQIGASSNQTVKATIGATQSSKIGLTRFETGGRISTSGEVQFTLKNYNGIDDFQFQKVVISTSVGTGLGALADEINKNADKTGVRATFTVETRGIAAVRAGATSDTFAINGVKIGKVDYKDGDANGALVAAINSVKDTTGVEASIDANGQLLLTSREGRGIKIDGNIGGGAFINADMKENYGRLSLVKNDGKDILISGSNLSSAGFGATQFISQASVSLRESKGQIDANIADAMGFGSANKGVVLGGYSSVSAYMSSAGSGFSSGSGYSVGSGKNYSTGFANAIAISAASQLSTVYNVSAGSGFSSGSTLSQFATMKTTAFGVKDETAGVTTLKGAMAVMDIAETAITNLDQIRADIGSVQNQVTSTINNITVTQVNVKAAESQIRDVDFAAESANYSKANILAQSGSYAMAQANSVQQNVLRLLQ

SEQ ID NO: 69 - protein, 364 aa, Haemophilus influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPAFFTDVNQMYDVLLNKSGATGVFTDFPDTGVEFLKGIK

SEQ ID NO: 70 - protein, 526 aa, Haemophilus influenzae
KAPMIDFSVVSRNGVATLVGDQYIVSVAHNGGYNSVDFGAEGPNPDQHRFTYQIVKRNNYKPGKDNPYHGDYHMPRLHKFVTDAEPIGMTTNMDGKVYANRNDYPERVRIGSGHQYWRTDKDEETNAYSSYDISGAYNYLIAGNTHTQSSGDNGTVHFSGNVIRPNHYGPLPIGGAQGDSGSPMFIYDAEKQKWFINGVLQTGHPFVGRGNGFQLIREEWFYTEVLAVDTPSVFRRYIPSINGHYSFVSNNDGTGKLTLTRPSKDGSKAKSEVGTVKLFNPSLEKTAKERAKAAPGYNIYQPRMEHGKNIYFGDRGTGTLTIENNINQGAGGLYFEGNFTVSSENNATWQGAGVHVSEDSTVTWKVNGVENDRLSKIGKGTLHVKAKGENKGSISVGDGKVILEQQADDQNKKQAFSEIGLVSGRGTVQLNDDKQFDTDKFYFGFRGGRLDLNGHSLTFKRIQNTDEGAMIVNHNTTQVANITITGNESIIAPTTKKNINKLDYSKEIAYNGWFGETDKNKHNGRL

SEQ ID NO: 71 - protein, 149 aa, Haemophilus influenzae
MKLTTQQTLKKGFTLIELMIVIAIIAILATIAIPSYQNYTKKAAVSELLQASAPYKADVELCVYSTGKPSSCSGGSNGIAADITTAKGYVKSVTTSNGAITVKGDGTLANMEYILQASGNAATGVTWTTTCKGTDASLFPANFCGSVTQ

SEQ ID NO: 72 - protein, 353 aa, Haemophilus influenzae
MKKTAIALVVAGLAAASVAQAAPQENTFYAGVKAGQASFHDGLRALAREKNVGYHRNSFTYGVFGGYQILNQNNLGLAVELGYDDFGRAKGREKGKTVAKHTNHGAHLSLKGSYEVLDGLDVYGKAGVALVRSDYKFYEDANGTRDHKKGRHTARASGLFAVGAEYAVLPELAVRLEYQWLTRVGKYRPQDKPNTAINYNPWIGSINAGISYRFGQGAAPVVAAPEVVSKTFSLNSDVTFAFGKANLKPQAQATLDSIYGEMSQVKSAKVAVAGYTDRIGSDAFNVKLSQERADSVANYFVAKGVAADAISATGYGKANPVTGATCDQVKGRKALIACLAPDRRVEIAVNGTK

SEQ ID NO: 73 - protein, 1098 aa, Haemophilus influenzae
MNKIFNVIWNVVTQTWVVVSELTRTHTKCASATVAVAVLATLLSATVEANNNTPVTNKLKAYGDANFNFTNNSIADAEKQVQEAYKGLLNLNEKNASDKLLVEDNTAATVGNLRKLGWVLSSKNGTRNEKSQQVKHADEVLFEGKGGVQVTSTSENGKHTITFALAKDLGVKTATVSDTLTIGGGAAAGATTTPKVNVTSTTDGLKFAKDAAGANGDTTVHLNGIGSTLTDTLVGSPATHIDGGDQSTHYTRAASIKDVLNAGWNIKGVKAGSTTGQSENVDFVHTYDTVEFLSADTETTTVTVDSKENGKRTEVKIGAKTSVIKEKDGKLFTGKANKETNKVDGANATEDADEGKGLVTAKDVIDAVNKTGWRIKTTDANGQNGDFATVASGTNVTFASGNGTTATVTNGTDGITVKYDAKVGDGLKLDGDKIAADTTALTVNDGKNANNPKGKVADVASTDEKKLVTAKGLVTALNSLSWTTTAAEADGGTLDGNASEQEVKAGDKVTFKAGKNLKVKQEGANFTYSLQDALTGLTSITLGTGNNGAKTEINKDGLTITPANGAGANNANTISVTKDGISAGGQSVKNVVSGLKKFGDANFDPLTSSADNLTKQNDDAYKGLTNLDEKGTDKQTPVVADNTAATVGDLRGLGWVISADKTTGGSTEYHDQVRNANEVKFKSGNGINVSGKTVNGRREITFELAKGEVVKSNEFTVKETNGKETSLVKVGDKYYSKEDIDLTTGQPKLKDGNTVAAKYQDKGGKVVSVTDNTEATITNKGSGYVTGNQVADAIAKSGFELGLADEADAKAAFDDKTKALSAGTTEIVNAHDKVRFANGLNTKVSAATVESTDANGDKVTTTFVKTDVELPLTQIYNTDANGKKITKVVKDGQTKWYELNADGTADMTKEVTLGNVDSDGKKVVKDNDGKWYHAKADGTADKTKGEVSNDKVSTDEKHVVSLDPNDQSKGKGVVIDNVANGDISATSTDAINGSQLYAVAKGVTNLAGQVNNLEGKVNKVGKRADAGTASALAASQLPQATMPGKSMVAIAGSSYQGQNGLAIGVSRISDNGKVIIRLSGTTNSQGKTGVAAGVGYQW

SEQ ID NO: 74 - protein, 1469 aa, Haemophilus influenzae
MNKIYRLKFSKRLNALVAVSELTRGCDHSTEKVSEKPVRTKVRHLALKPLSAILLSLGIVSIPQSVLASGLQGMSVVHGTATMQVDGNKTTIRNSVNAIINWKQFNIDQNEMVQFLQENNNSAVFNRVTSDQISQLKGILDSNGQVFLINPNGITIGKDAIINTNGFTASTLDISNENIKARNFTLEQTKDKALAEIVNHGLITVGKDGSVNLIGGKVKNEGVISVNGGSISLLAGQKITISDIINPTITYSIAAPENEAINLGDIFAKGGNINVRAANIRNKGKLSADSVSKDKSGNIVLSAKEGEAEIGGVISAQNQQAKGGKLMITGDKVTLKTGAVIDLSGKEGGETYLGGDERGEGKNGIQLAKKTSLEKGSTINVSGKEKGGRAIVWGDIALIDGNINARGSDITKTGGFVETSGHDLFIKDNAIVDAKEWLLDPDEVSINALALGRTDIPQESSEYTLESASNEPERKKNKLNLDTLTNATLENILKRGTSVNITATRKINVTSAINLSNGSLTLHTERGGIELNGDITSDNNKAGANLTIKSGGWLDIHSNISLGERDNINITAKGDIAFEKGNNQIIKGRGVITSGDQNGFRFDNVSLNGVGTGLLFNTKRSKNGNGNISNHFRGTLNISGIVNISMAAPNTSWLRRVYGRTYWNLTSLNVAENSKFNLTIDNRGSGTAGTLNRRSNLNGITFNGDTTFNVSSGSMVNFDIKASIIVPRSFDVNYALFNGNVSVLGGGRVAFNLDASSNSFETSGVTIKSHFFNVSGGSTLNLSTNGSTKTAFSIENDLTLNATGSSITLKQIEGTDSRIGNGVVANKNITFNGGNITLGSQKAPTQIKGNVTINANTNATLRGANFNNNKSALNITGNVINNGNLTTDGSIINIGGSLTVAESAKLQAVTNFTFNVAGLFDNKGNSNISIAKGGAKFKDINNTKNLSIATNSSAAYRTIIAGNITNKSGDLNITDNKSDAEIEIGGDISQKEGNLTISSDKINITKQITIKKDIDVKSSDSDVTANLTIKTKELKLAGDLNISNFDKAEVVAKGNNDLTIGNSDGNGNAEAKTVTFNNVNESKISADGHNVTLNSKVETSGNNGGAKSNSNNDTGLTITAKNVEVNKDIASHKTVNITASENVTTKAGTTINATTGNVKLTAVTSDIQGGIKSNSGDVNITTSTGSINGKIESKSGSVTIAASGDTLNVSNVSGNAVTITADKGKLTAQAGSTIQATTGEANITSATGTIDGTVSGGTVNVTASSGSLTVGGDAKINATEGAATLTATKGTLTTVKGSNIDANKGTLVINAKLDGEASGNRTEVNATNASGSGSVTAATSSSVNITGDLNTINGLNIISENGRNTVRLRGKEIDVKYIQPGVASVEEVIEAKRVLEKVKDLSDEERETLAKLGVSAVRFVEPNNAITVNTQNEFTTRPSSQVTISEGKACFSSGNGAAVCTNVADDGQP

SEQ ID NO: 75 - protein, 905 aa, Bordetella pertussis
MNMSLSRIVKAAPLRRTTLAMALGALGAAPAAHADWNNQSIVKTGERQHGIHIQGSDPGGVRTASGTTIKVSGRQAQGILLENPAAELQFRNGSVTSSGQLSDDGIRRFLGTVTVKAGKLVADHATLANVGDTWDDDGIALYVAGEQAQASIADSTLQGAGGVQIERGANVTVQRSAIVDGGLHIGALQSLQPEDLPPSRVVLRDTNVTAVPASGAPAAVSVLGASELTLDGGHITGGRAAGVAAMQGAVVHLQRATIRRGDAPAGGAVPGGAVPGGFGPGGFGPVLDGWYGVDVSGSSVELAQSIVEAPELGAAIRVGRGARVTVSGGSLSAPHGNVIETGGARRFAPQAAPLSITLQAGAHAQGKALLYRVLPEPVKLTLTGGADAQGDIVATELPSIPGTSIGPLDVALASQARWTGATRAVDSLSIDNATWVMTDNSNVGALRLASDGSVDFQQPAEAGRFKVLTVNTLAGSGLFRMNVFADLGLSDKLVVMQDASGQHRLWVRNSGSEPASANTLLLVQTPLGSAATFTLANKDGKVDIGTYRYRLAANGNGQWSLVGAKAPPAPKPAPQPGPQPPQPPQPQPEAPAPQPPAGRELSAAANAAVNTGGVGLASTLWYAESNALSKRLGELRLNPDAGGAWGRGFAQRQQLDNRAGRRFDQKVAGFELGADHAVAVAGGRWHLGGLAGYTRGDRGFTGDGGGHTDSVHVGGYATYIADSGFYLDATLRASRLENDFKVAGSDGYAVKGKYRTHGVGASLEAGRRFTHADGWFLEPQAELAVFRAGGGAYRAANGLRVRDEGGSSVLGRLGLEVGKRIELAGGRQVQPYIKASVLQEFDGAGTVHTNGIAHRTELRGTRAELGLGMAAALGRGHSLYASYEYSKGPKLAMPWTFHAGYRYSW

SEQ ID NO: 76 - protein, 1055 aa, Escherichia coli
MLFCTCRYPHLWVPAFLFTLCKEKLMKRHLNTSYRLVWNHITGTLVVASELARSRGKRAGVAVALSLAAVTSVPALAADTVVQAGETVNDGTLTNHDNQIVFGTANGMTISTGLEYGPDNEANTGGQWIQNGGTANNTTVTGGGLQRVNPGGSVSDTVISAGGGQSLQGQAVNTTLNGGEQWVHEDGIATGTVINEKGWQAIKSGAVATDTVVNTGAEGGPDAENGDTGQTVYGDAVRTTINKNGRQIVAAEGTANTTVVYAGGDQTVHGHALDTTLNGGYQYVHNGGTASGTVVNSDGWQIVKNGGVAGNTTVNQKGRLQVDAGGTATNVTLKQGGALVTSTAATVTGINRLGAFSVVEGKADNVVLENGGRLDVLTGHTATNTRVDDGGTLDVRNGGTATTVSMGNGGVLLADSGAAVSGTRSDGKAFSIGGGQADALMLEKGSSFTLNAGDTATDTTVNGGLFTARGGTLAGTTTLNNGAILTLSGKTVNNDTLTIREGDALLQGGSLTGNGSVEKSGSGTLTVSNTTLTQKAVNLNEGTLTLNDSTVTTDVIAQRGTALKLTGSTVLNGAIDPTNVTLASGATWNIPDNATVQSVVDDLSHAGQIHFTSTRTGKFVPATLKVKNLNGQNGTISLRVRPDMAQNNADRLVIDGGRATGKTILNLVNAGNSASGMATTGKGIQVVEAINGATTEEGAFVQGNKLQAGAFNYSLNRDSDESWYLRSENAYRAEVPLYASMLTQAMDYDRILAGSRSHQTGVNGENNSVRLSIQGGHLGHDNNGGIARGATPESSGSYGFVRLEGDLLRTEVAGMSVTAGVYGAAGHSSVDVKDDDGSRAGTVRDDAGSLGGYLNLIHNASGLWADIVAQGTRHSMKASSDNNDFRARGWGWLGSLETGLPFSITDNLMLEPQLQYTWQGLSLDDGKDNAGYVKFGHGSAQHVRAGFRLGSHNDMTFGEGTSSRDTLRGSAKHSVRELPVNWWVQPSVIRTFSSRGDMSMGTAAAGSNMTFSPSQNGTSLDWKPVSGKISPWAFRQVMSTASAAAALKAITVRPR

SEQ ID NO: 77 - protein, 567 aa, Helicobacter pylori
MISRKEYVSMYGPTTGDKVRLGDTDLIAEVEHDYTIYGEELKFGGGKTLREGMSQSNNPSKEELDLIITNALIVDYTGIYKADIGIKDGKIAGIGKGGNKDMQDGVKNNLSVGPATEALAGEGLIVTAGGIDTHIHFISPQQIPTAFASGVTTMIGGGTGPADGTNATTITPGRRNLKWMLRAAEEYSMNLGFLAKGNTSNDASLADQIEAGAIGFKIHEDWGTTPSAINHALDVADKYDVQVAIHTDTLNEAGCVEDTMAAIAGRTMHTFHTEGAGGGHAPDIIKVAGEHNILPASTNPTIPFTVNTEAEHMDMLMVCHHLDKSIKEDVQFADSRIRPQTIAAEDTLHDMGIFSITSSDSQAMGRVGEVITRTWQTADKNKKEFGRLKEEKGDNDNFRIKRYLSKYTINPAIAHGISEYVGSVEVGKVADLVLWSPAFFGVKPNMIIKGGFIALSQMGDANASIPTPQPVYYREMFAHHGKAKYDANITFVSQAAYDKGIKEELGLERQVLPVKNCRNITKKDMQFNDTTAHIEVNPETYHVFVDGKEVTSKPATKVSLAQLFSIF

SEQ ID NO: 78 - protein, 391 aa, Neisseria meningitidis
MKHFPSKVLTTAILATFCSGALAATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALDATTNALNKLGENITTFAEETKTNIVKIDEKLEAVADTVDKHAEAFNDIADSLDETNTKADEAVKTANEAKQTAEETKQNVDAKVKAAETAAGTANTAADKAEAVAAKVTDIKADIATNKDNIAKKANSADVYTREESDSKFVRIDGLNATTEKLDTRLASAEKSITEHGTRLNGLDRTVSDLRKETRQGLAEQAALSGLFQPYNVGRFNVTAAVGGYKSESAVAIGTGFRFTENFAAKAGVAVGTSSGSSAAYHVGVNYEW

SEQ ID NO: 79 - protein, 274 aa, Neisseria meningitidis
MNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 80 - protein, 674 aa, Neisseria meningitidis
ALDTVTVKGDRQGSKIRTNIVTLQQKDESTATDMRELLKEEPSIDFGGGNGTSQFLTLRGMGQNSVDIKVDNAYSDSQILYHQGRFIVDPALVKVVSVQKGAGSASAGIGATNGAIIAKTVDAQDLLKGLDKNWGVRLNSGFASNEGVSYGASVFGKEGNFDGLFSYNRNDEKDYEAGKGFRNVNGGKTVPYSALDKRSYLAKIGTTFGDDDHRIVLSHMKDQHRGIRTVREEFTVGDKSSRINIDRQAPAYRETTQSNTNLEYTGKNLGFVEKLDANAYVLEKKRYSADDKGSGYAGNVEGPNHTRITTRGMNFNFDSRLAEQTLLKYGINYRHQEIKPQAFLNGDFTISSKKKDPNNPKNEIDKDPTEIAKDKEDMALVHSYKLSNPTKTDAGVYVEAIHDIGDFTLTGGLRYDRFKVKTHDGKTVSSSSLNPSFGVIWQPHEHWSFSASHNYASRSPRLYDALQTHGKRGIISIADGTKAERARNTEIGFNYNDGTFAANGSYFWQTIKDALANPQNRHDSVAVREAVNAGYIKNHGYELGASYRTGGLTAKVGVSYSKPRFYGYDTHKDKLLSANPEFGAQVGRTWTASLAYRFQNPNLEIGWRGRYVQKAVGSILVSGQKDRQGNLENVVRKGFGVNDVFANWKPLGKDTLNVNLSVNNVFNKYYYPHS

SEQ ID NO: 81 - protein, 272 aa, Plasmodium falciparum
MWIVKFLIVVHFFIICTINFDKLYISYSYNIVPENGRMLNMRILGEEKPNVDGVSTSNTPGGNESSSASPNLSDAAEKKDEKEASEQGEESHKKENSQESANGKDDVKEEKKTNEKKDDGKTDKVQEKVLEKSPKESQMVDDKKKTEAIPKKVVQPSSSNSGGHVGEEEDHNEGEGEHEEEEEHEEDDDDEDDDTYNKDDLEDEDLCKHNNGGCGDDKLCEYVGNRRVKCKCKEGYKLEGIECVELLSLASSSLNLIFNSFITIFVVILLIN

SEQ ID NO: 82 - protein, 272 aa, Plasmodium falciparum
MNILCILSYIYFFVIFYSLNLNNKNENFLVVRRLMNDEKGEGGFTSKNKENGNNNRNNENELKEEGSLPTKMNEKNSNSSDKQPNDISHDESKSNSNNSQNIQKEPEEKENSNPNLDSSENSSESATRSVDISEHNSNNPETKEENGEEPLDLEINENAEIGQEPPNRLHFDNVDDEVPHYSALRYNKVEKNVTDEMLLYNMMSDQNRKSCAINNGGCSDDQICININNIGVKCICKDGYLLGTKCIILNSYSCHPFFSILIYITLFLLLFV

SEQ ID NO: 83 - protein, 330 aa, Staphylococcus aureus
VAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMPAYIDPENVKKTGNVTLATGIGSTTANKTVLVDYEKYGKFYNLSIKGTIDQIDKTNNTYRQTIYVNPSGDNVIAPVLTGNLKPNTDSNALIDQQDTSIKVYKVDNAADLSESYFVNPENFEDVTNSVNITFPNPNQYKVEFNTPDDQITTPYIVVVNGHIDPNSKGDLVLRSTLYGYNSNIIWRSMSWDNEVAFNNGSGSGDGIDKPVVPEQPDE

SEQ ID NO: 84 - protein, 434 aa, Staphylococcus aureus
MPIITDVYAREVLDSRGNPTVEVEVLTESGAFGRALVPSGASTGEHEAVELRDGDKSRYLGKGVTKAVENVNEIIAPEIIEGEFSVLDQVSIDKMMIALDGTPNKGKLGANAILGVSIAVARAAADLLGQPLYKYLGGFNGKQLPVPMMNIVNGGSHSDAPIAFQEFMILPVGATTFKESLRWGTEIFHNLKSILSQRGLETAVGDEGGFAPKFEGTEDAVETIIQAIEAAGYKPGEEVFLGFDCASSEFYENGVYDYSKFEGEHGAKRTAAEQVDYLEQLVDKYPIITIEDGMDENDWDGWKQLTERIGDRVQLVGDDLFVTNTEILAKGIENGIGNSILIKVNQIGTLTETFDAIEMAQKAGYTAVVSHRSGETEDTTIADIAVATNAGQIKTGSLSRTDRIAKYNQLLRIEDELFETAKYDGIKSFYNLDK

SEQ ID NO: 85 - protein, 246 aa, Staphylococcus aureus
MKMTKSALVTGASRGIGRSIALQLAEEGYNVAVNYAGSKEKAEAVVEEIKAKGVDSFAIQANVADADEVKAMIKEVVSQFGSLDVLVNNAGITRDNLLMRMKEQEWDDVIDTNLKGVFNCIQKATPQMLRQRSGAIINLSSVVGAVGNPGQANYVATKAGVIGLTKSAARELASRGITVNAVAPGFIVSDMTDALSDELKEQMLTQIPLARFGQDTDIANTVAFLASDKAKYITGQTIHVNGGMYM

SEQ ID NO: 86 - protein, 306 aa, Staphylococcus aureus
MIRNRVMNSVVNKYLLHNRSIMFKNDQDVERFFYKREIENRKKHKQPSTLNVKANLEKLSLDDMQVFRFNFRHQIDKKILYIHGGFNALQPSPFHWRLLDKITLSTLYEVVLPIYPKTPEFHIDDTFQAIQRVYDQLVSEVGPQNVVVMGDGSGGALALSFVQSLLDNQQPLPNKLYLISPILDATLSNKDISDALIEQDAVLSQFGVNEIMKKWANGLPLTDKRISPINGTIEGLPPVYMFGGGREMTHPDMKLFEQMMLQHHQYIEFYDYPKMVHDFPIYPIRQSHKAIKQIAKSIDDDVTQNN

SEQ ID NO: 87 - protein, 613 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQT

SEQ ID NO: 88 - protein, 241 aa, Staphylococcus aureus
MKKNITNKIVLSTALLLLGTTSTQPPKAPISFSSEAKAYNISENETNINELIKYYTQPHLSFSGKWLWQNPNGTIHATLQTWVWYTHIQVFGPESWGNINQLRDKYVDIFGTKDEDTIEGYWTYDETFTGGVTPAATSSDKPYKLFLKYKDKQQTMIGGLEFYQGNKPVITLKELDFRVRQTLIKNKKLYNGEFNKGQIRITGGGNHYTIDLSKKLKLTDTNSYVKNPRHAEIEVILEKSH

SEQ ID NO: 89 - protein, 273 aa, Clostridium difficile
ATTGTQGYTVVKNDWKKAVKQLQDGLKDNSIGKITVSFNDGVVGEVAPKSANKKADRDAAAEKLYNLVNTQLDKLGDGDYVDFSVDYNLENKIITNQADAEAIVTKLNSLNEKTLIDIATKDTFGMVSKTQDSEGKNVAATKALKVKDVATFGLKSGGSEDTGYVVEMKAGAVEDKYGKVGDSTAGIAINLPSTGLEYAGKGTTIDFNKTLKVDVTGGSTPSAVAVSGFVTKDDTDLAKSGTINVRVINAKEESIDIDASSYHMSLEHHHHHH

SEQ ID NO: 90 - protein, 126 aa, Clostridium difficile
ILQRMRTLSLQSSNETNTAEERQKVADELLQLKDEVERISSSIEFNGKKLLDGSSTEIRLQVGANFGTNVAGTTNNNNEIKVALVNTSSIMSKAGITSSTIASLNVDGASGTDAAKQMVSSLDMAL

SEQ ID NO: 91 - protein, 507 aa, Clostridium difficile
MSSISPVRVTGLSGNFDMEGIIEASMIRDKEKVDKAKQEQQIVKWKQEIYRNVIQESKDLYDKYLSVNSPNSIVSEKAYSSTRITSSDESIIVAKGSAGAEKINYQFAVSQMAEPAKFTIKLNSSEPIVRQFPPNASGASSLTIGDVNIPISEQDTTSTIVSKINSLCADNDIKASYSEMTGELIISRKQTGSSSDINLKVIGNDNLAQQIANDNGITFANDASGNKVASVYGKNLEADVTDEHGRVTHISKEQNSFNIDNIDYNVNSKGTAKLTSVTDTEEAVKNMQAFVDDYNKLMDKVYGLVTTKKPKDYPPLTDAQKEDMTTEEIEKWEKKAKEGILRNDDELRGFVEDIQSAFFGDGKNIIALRKLGINESENYNKKGQISFNADTFSKALIDDSDKVYKTLAGYSSNYDDKGMFEKLKDIVYEYSGSSTSKLPKKAGIEKTASASENVYSKQIAEQERNISRLVEKMNDKEKRLYAKYSALESLLNQYSSQMNYFSQAQGN

SEQ ID NO: 92 - protein, 191 aa, Clostridium difficile
YNLKSEAQGATKPSNMDTAPTQFNVTDVVRLNKDKETVKNAIMQYGSVTSGYAHYSTYFNKDETAYNCTNKRAPLNHAVAIVGWDDNYSKDNFASDVKPESNGAWLVKSSWGEFNSMKGFFWISYEDKTLLTDTDNYAMKSVSKPDSDKKMYQLEYAGLSKIMSNKVTAANVFDFSRDSEKLDSVMFETDS

SEQ ID NO: 93 - protein, 610 aa, Clostridium difficile
MKISKKIVSLLTMTFLTVTLYGNTSNASTKDTLTGSGRWETAIKISQAGWTKSESAVLVNDNSIADALSATPFAKAKDAPILLTQSNKLDSRTKAELKRLGVKNVYLIGGSIALSSEIEKQLNAENINFERISGNSRYDTSLKLAEKLDREKSISKIVVVNGEKGLADAVSVGAIAAQENMPIILSDSENGTEVADNFIDSKDIAKSYVIGGTYSISSSVERSLPNATRIAGSSRSETNAKIIEEFYKDTDIKNIYVTKDGTKNKNDLIDSLAVGVLAAKNSSPIILAGNKLDTTQKDVLNTKIIDKVTQIGGLGNEDAVKSIVDMQEKTKYTVETIEELNVAIKKADANDVIIFEPEKDTNISDSFKIATNKAITVEFDGVFKQSITIDMPNGDVKNFGEISDDIRIDNIKKGTLINEGSIQGIDIYSKNGCKIENTSDGDIWIITIDADAKDVYIENDGDITKISNNAPGVIIKNSGKIDLVNGNEQPAISGKKPTTNDTEYNDERARGLSVSTKPCSIPEKNRVRVTISSEPKSSRYKIYYRVVEDKPSAMYVGEKISVRSWDLASKSDGSFVEKAKNGSYIEVVEINTSTNKVSRWGRSNVTDDGF

SEQ ID NO: 94 - protein, 409 aa, Clostridium difficile
LYTSLININTNYYSNEYYPEIIVLNPNTFHKKVNINLDSSSFEYKWSTEGSDFILVRYLEESNKKILQKIRIKGILSNTKSFNKMSIDFKDIKKLSLGYIMSNFKSFNSENELDRDHLGFKIIDNKTYYYDEASKLVKGLININNSLFYFDPIESNLVTGWQTINGKKYYFDINTGAASTSYKIINGKHFYFNNDGVMQLGVFKGPDGFEYFAPANTQNNNIEGQAIVYQSKFLTLNGKKYYFDNDSKAVTGWQTIDGKKYYFNLNTAEAATGWQTIDGKKYYFNTNTFIASTGYTIINGKHFYFNTDGIMQIGVFKGPNGFEYFAPANTDANNIEGQAIRYQNRFLYLHDNIYYFGNNSKAVTGWQTINGKVYYFMPDTGMAAAGGLFEIDGVIYFFGVDGVKAPGIY

SEQ ID NO: 95 - protein, 100 aa, Mycobacterium tuberculosis
MAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 96 - protein, 338 aa, Mycobacterium tuberculosis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNAVPAPQGA

SEQ ID NO: 97 - protein, 325 aa, Mycobacterium tuberculosis
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAANMWGPSSDPAWERNDPTQQIPKLVANNTRLWVYCGNGTPNELGGANIPAEFLKNFVRSSNLKFQDAYNAAGGHNAVFNFPPNGTHSWEYWGAQLNAMKGDLQSSLGAG

SEQ ID NO: 98 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 99 - protein, 422 aa, artificial
  botulinum toxin synthetic construct
MYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN